(19) United States
(12) Patent Application Publication
(10) Pub. No.: US 2016/0040155 A1
(43) Pub. Date: Feb. 11, 2016

(54) ACTIVATING AN ALTERNATIVE PATHWAY FOR HOMOLOGY-DIRECTED REPAIR TO STIMULATE TARGETED GENE CORRECTION AND GENOME ENGINEERING

(71) Applicant: UNIVERSITY OF WASHINGTON THROUGH ITS CENTER FOR COMMERCIALIZATION, Seattle, WA (US)

(72) Inventors: Nancy MAIZELS, Seattle, WA (US); Luther DAVIS, Seattle, WA (US)

(21) Appl. No.: 14/776,835

(22) PCT Filed: Apr. 16, 2014
(86) PCT No.: PCT/US14/34364

§ 371 Date: Sep. 15, 2015

Related U.S. Application Data
(60) Provisional application No. 61/812,498, filed on Apr. 16, 2013.
(60) Provisional application No. 61/909,328, filed on Nov. 26, 2013.
(60) Provisional application No. 61/932,709, filed on Jan. 28, 2014.

(51) Int. Cl. C12N 15/10

(52) U.S. Cl. CPC C12N 15/102; A61K 48/00

(57) ABSTRACT

The technology described herein is directed to methods for modulating the rate of homology-directed repair, e.g. in methods for gene modification.

### CROSS-REFERENCE TO RELATED APPLICATIONS

This application claims benefit under 35 U.S.C. §119(e) of U.S. Provisional Application No. 61/812,498 filed Apr. 16, 2013; 61/909,328 filed Nov. 26, 2013; and 61/932,709 filed Jan. 28, 2014, the contents of which are incorporated herein by reference in their entirety.

### GOVERNMENT SUPPORT

This invention was made with government support under Grant No. GM RL1 084434, awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy, created on Apr. 11, 2014, is named 034186-080730-PCT_SL.txt and is 986,323 bytes in size.

### TECHNICAL FIELD

The technology described herein relates to modulation of DNA repair mechanisms and applications thereof, e.g., gene modification, gene therapy, treatment of cancer and/or infectious disease.

### BACKGROUND

The DNA present in cells can be damaged by toxins, radiation, and/or oxidative stress. The most common form DNA damage severes a single strand of the phosphodiester backbone of the DNA double helix. This is referred to as a nick. If both strands are severed, the damage is referred to as a double-strand break (DSB).

Cells have a number of different mechanisms to repair the various forms of DNA damage, e.g. base excision repair, nucleotide excision repair, mismatch repair, single-strand break repair, and double-strand break repair. One type of repair is referred to as homology-directed repair (HDR). HDR uses a template molecule with homology to the damaged DNA to effect the repair and can occur at both nicks and DSBs.

### SUMMARY

As described herein, the inventors have discovered that there are in fact two types of HDR. The first, “canonical HDR,” has been well studied at DSBs, using as the donor for repair a double-strand DNA (dsDNA) molecule. Canonical HDR is positively regulated by RAD51, BRCA2 and BRCA1.

Described herein is a new type of HDR, “alternative HDR”. Notably, alternative HDR is suppressed by the activity of BRCA2 and functionally related genes (see, e.g., Table 1). Alternative HDR can use single-stranded DNA (ssDNA) or nicked dsDNA donor molecules. Efficiency of alternative HDR at nicks can reach levels comparable to canonical HDR at DSBs, but the rate of mutagenesis (i.e. incorrect repair) is much lower. Accordingly, described herein are improved gene correction and genome engineering methods that utilize alternative HDR in order to permit efficient gene modification with reduced rates of accompanying mutagenesis.

In one aspect, described herein is a method of increasing alternative homology-directed repair (HDR) at a target nucleic acid nick in a cell, the method comprising contacting the cell with an inhibitor of RAD51; BRCA2; PALB2 or SHFM1.

In one aspect, described herein is a method of modifying the sequence of a target nucleic acid molecule, the method comprising contacting the target nucleic acid molecule with a) a donor nucleic acid molecule comprising the modification to be made in the target nucleic acid molecule; b) a nickase; and c) an inhibitor of RAD51; BRCA2; PALB2 or SHFM1.

In some embodiments of the foregoing aspects, a cell-free system comprises the target nucleic acid molecule. In some embodiments of the foregoing aspects, a cell comprises the target nucleic acid molecule. In some embodiments of the foregoing aspects, the rate of mutagenic end joining is not increased. In some embodiments of the foregoing aspects, the rate of mutagenic end joining is not altered. In some embodiments of the foregoing aspects, the method further comprises generating a nick in the transcribed strand of the target nucleic acid molecule. In some embodiments of the foregoing aspects, the nickase is selected from the group consisting of: a nuclease with one active site disabled; I-AniI with one active site disabled; or Cas9^{D10A}. In some embodiments of the foregoing aspects, the donor nucleic acid molecule is a ssDNA or nicked dsDNA. In some embodiments of the foregoing aspects, the donor nucleic acid molecule comprises a portion complementary to the strand of the target nucleic acid molecule that is not nicked by the nickase. In some embodiments of the foregoing aspects, the portion of the donor nucleic acid molecule that is complementary to a strand of the target nucleic acid molecule is substantially centered with respect to the location of the nick.

In one aspect, described herein is a method of modifying the sequence of a target nucleic acid molecule, the method comprising contacting the target nucleic acid molecule with a) a ssDNA donor nucleic acid molecule comprising the modification to be made in the target nucleic acid molecule; b) a nuclease; and c) an inhibitor of RAD51; BRCA2; PALB2 or SHFM1. In some embodiments, a cell-free system comprises the target nucleic acid molecule. In some embodiments, a cell comprises the target nucleic acid molecule. In some embodiments, the rate of mutagenic end joining is not increased. In some embodiments, the rate of mutagenic end joining is not altered. In some embodiments, the donor nucleic acid molecule comprises a portion complementary to one strand of the target nucleic acid molecule. In some embodiments, the nuclease is selected from the group consisting of: nucleases comprising a FokI cleavage domain; zinc finger nucleases; TALE nucleases; RNA-guided engineered nucleases; Cas9; Cas9-derived nucleases; and homing endonucleases.

In some embodiments of the foregoing aspects, the modification is introduced as a gene therapy. In some embodiments of the foregoing aspects, the inhibitor is an inhibitory nucleic acid. In some embodiments of the foregoing aspects, the inhibitor is an antibody reagent. In some embodiments of the foregoing aspects, the inhibitor is selected from the group consisting of: IBR2; RI-1; RI-2; and B02. In some embodiments of the foregoing aspects, the donor nucleic acid molecule is at least about 25 nt in length. In some embodiments of the foregoing aspects, the donor nucleic acid molecule is at least about 50 nt in length. In some embodiments of the foregoing aspects, the method further comprises the step of implanting a cell comprising the modified nucleic acid molecule into a subject. In some embodiments of the foregoing aspects, the cell is autologous to the subject. In some embodiments of the foregoing aspects, the cell is an iPS cell. In some embodiments of the foregoing aspects, the modification corrects a mutation. In some embodiments of the foregoing aspects, the modification introduces a mutation. In some embodiments of the foregoing aspects, the modification causes improved cell function. In some embodiments of the foregoing aspects, the modification is selected from the group consisting of: modification of a viral gene and modification of a gene comprising a dominant negative mutation.

In one aspect, described herein is a method of decreasing genomic instability in a cell, the method comprising contacting the cell with an agonist of RAD51; BRCA2; PALB2 or SHFM1 or an inhibitor of BRCA1. In some embodiments, the agonist is a nucleic acid encoding RAD51; BRCA2; PALB2 or SHFM1. In some embodiments, the inhibitor is an inhibitory nucleic acid. In some embodiments, the inhibitor is an antibody reagent. In some embodiments, the cell is a cancerous cell. In some embodiments, the genomic instability is a loss of heterozygosity. In some embodiments, the contacting step comprises administering the agonist or inhibitor to a subject in need of treatment for a risk of genomic instability. In some embodiments, the subject is a subject with cancer.

In one aspect, described herein is a kit comprising: a nuclease or a nickase; and an inhibitor of RAD51; BRCA2; PALB2 or SHFM1. In some embodiments, the kit can further comprise a donor nucleic acid molecule. In some embodiments, the donor nucleic acid molecule is a single-stranded nucleic acid molecule. In some embodiments, the nickase is selected from the group consisting of: a nuclease with one active site disabled; I-AniI with one active site disabled; or Cas9^{D10A}. In some embodiments, the nuclease is selected from the group consisting of nucleases comprising a FokI cleavage domain; zinc finger nucleases; TALE nucleases: RNA-guided engineered nucleases; Cas9; Cas9-derived nucleases: and homing endonucleases. In some embodiments, the inhibitor is an inhibitory nucleic acid. In some embodiments, the inhibitor is an antibody reagent. In some embodiments, the inhibitor is selected from the group consisting of IBR2; RI-1; RI-2; and B02. In some embodiments, the kit can further comprise a cell extract.

### BRIEF DESCRIPTION OF THE DRAWINGS

FIGS. 1A-1F depicts efficient HDR at transcribed strand nicks. FIG. 1A depicts a diagram of the TL^{TP} and TL^{NT} reporters, nicked by I-AniI nickase on the transcribed (orange, labeled “bottom”; and non-transcribed strands (blue, labeled top) strands; nicks are indicated by arrowhead). FIG. 1B depicts a diagram of the chromosomal I-AniI Traffic Light (TL) reporter⁹ consisting of a defective GFP gene containing an I-AniI site (arrowheads) and two stop codons (asterisks) near the 5′-end, joined by a T2A translational linker to the mCherry coding sequence in the +2 open reading frame (ORF). The I-AniI site is oriented to nick the transcribed strand in the TL^{TP} reporter and the non-transcribed strand in the TL^{NT} reporter, as indicated by the two arrowheads. GFP+ cells will result from HDR of the chromosomal reporter using an exogenous donor; while mCherry+ cells will result from cleavage at the I-AniI site followed by mutagenic end joining that puts mCherry in the +2 reading frame. FIG. 1C depicts graphs of a representative flow assay of HDR (GFP+) and mutagenic end joining (EJ) (mCherry+) cells generated following transient transfection of 293T-TL^{TP} and 293T-TL^{NT} cells with constructs expressing catalytically inactive I-AniI (I-AniIDEAD) or I-AniI nickase (Nick) or cleavase (DSB), and the dsDNA plasmid donor pCS14GFP, which bears a 3′-truncated defective GFP gene and 2.47 kb of upstream and 0.56 kb of downstream homology with the chromosomal reporter. FIG. 1D depicts graphs of HDR (GFP+) frequencies following multiple independent transient transfections of 293T-TL^{TP} or 293T-TL^{NT} cells, one set of which is shown in panel Cc; n=2 (I-AniIDEAD) or 4 (nicks and DSBs). At DSBs, the difference between HDR frequencies at the TL^{TP} and TL^{NT} reporters are statistically significant at nicks (p<0.005) but not statistically significant at DSBs (p=0.23). FIG. 1E depicts a diagram of the chromosomal P-Tet-TL reporter, which carries a tetracycline-inducible promoter (P-Tet) upstream of the defective GFP repair target. FIG. 1F depicts graphs of normalized HDR frequencies, based on pooled results from a total of 11 independent transfections of three different clonal 293-P-Tet-TL^{TP} or 293-P-Tet-TL^{NT} cell lines carried out in the absence (OFF) or presence (ON) of 1 μg/ml doxycycline. Frequencies for each line were normalized to the OFF frequency independently for nicks and DSBs. Analyses of individual lines are shown in FIG. 7. FIGS. 1D and 1F present the mean and standard error of the mean (SEM); * and ** indicates p<0.05 and p<0.005, respectively.

FIGS. 2A-2C demonstrate donor use and donor strand bias of HDR at nicks. FIG. 2A depicts a diagram of TL^{TP} and TL^{NT} reporters, and 99 nt ssDNA oligonucleotide donors 99-TOP and 99-BOT centered at the nick and identical to top or bottom strand except for the indicated 17 nt region of heterology for replacement of the stop codons and I-AniI site with GPF coding sequence. Lettering is color-coded to match transcribed (stippled) or non-transcribed (white) strands. FIG. 2B depicts graphs of HDR (GFP+) frequencies at nicks or DSBs, using the 99-TOP or 99-BOT ssDNA donors, in 293T-TL^{TP}-7 and HT1080 TL^{TP}-4 clonal lines. FIG. 2C depicts graphs of HDR (GFP+) frequencies at nicks or DSBs, using the 99-TOP or 99-BOT ssDNA donors, in 293T-TL^{NT} and 293T-TL^{TP} cell populations. FIGS. 2B and 2C present the mean and SEM of at least five transfections; * and ** indicate p<0.05 and p<0.005, respectively.

FIGS. 3A-3H demonstrate that downregulation or inhibition of RAD51 stimulates HDR at nicks by ssDNA donors. FIGS. 3A-3B depict graphs of HDR (GFP+) frequencies at nicks or DSBs, using the pCS14GFP dsDNA donor or 99-TOP or 99-BOT ssDNA donors, in the 293T-TL^{TP}-7 clonal line treated with siRAD51 (FIG. 3A) or transiently expressing the RAD51K133R dominant negative mutant (FIG. 3B). FIG. 3C depicts graphs of HDR (GFP+) frequencies at nicks, using the ds DNA donor or 99-TOP or 99-BOT ss DNA donors, in 293T-TL^{NT} and 293T-TL^{TP} cell populations. FIG. 3D depicts graphs of HDR (GFP+) frequencies at nicks or DSBs in the 293T-TL^{TP}-7 clonal line treated with siBRCA2, using ds or BOT donors described in FIG. 3A. FIG. 3E depicts graphs of mutagenic EJ (mCherry+) frequencies at nicks or DSBs, using the dsDNA donor or 99-TOP or 99-BOT ssDNA donors, in the 293T-TL^{TP}-7 clonal line transiently expressing the RAD51K133R dominant negative mutant. FIG. 3F depicts a graph of the ratio of HDR to mutagenic EJ (% GFP:% mCherry cells) in the 293T-TL^{TP}-7 clonal line. Data were compiled from transfections carried out under optimal HDR conditions for nicks (transcribed-strand nick, bottom strand donor, RAD51 inhibited) or DSBs (dsDNA donor). FIG. 3G depicts a diagram of donors used to compare HDR by intact (pG-no) dsDNA or dsDNA carrying an I-AniI site oriented for nicking the transcribed (pGAn-TP) or non-transcribed (pGAn-NT) DNA strand. The I-AniI site in pGAn-TP and pGAn-NT is approximately 500 bp downstream of the I-AniI site in the reporter GFP gene targeted for repair. These donors contain 0.1 kb of upstream and 0.49 kb of downstream homology with the chromosomal reporter (see FIGS. 5A-5E). FIG. 3H depicts graphs of HDR (GFP+) frequencies at nicks, using the dsDNA donors shown in FIG. 3G which lack I-AniI sites or carry a site oriented for nicking the non-transcribed (NT) or transcribed (TP) DNA strand. Mean and SEM of at least five transfections; * and ** indicate p<0.05 and p<0.005, respectively. FIGS. 4A-4B depict pathways of HDR at nicks.

FIGS. 4A-4B depict diagrams of TL^{TP} and TL^{NT} reporters, and 75 nt ssDNA donors with 17 nt region of heterology, and flanking sequences of indicated lengths identical to top (white) or bottom (stippled) strand. Pooled 293T cells bearing the either the TL^{TP} or TL^{NT} reporter, as indicated, and transiently expressing RAD51K133R, were provided with a 75 nt donor centered at the nick (black) or extending to either side of the nick, as shown. Graphs show HDR frequencies normalized to the donor centered at the nick, and represent mean and SEM of at least seven transfections. FIG. 4B depicts diagrams of the working model for repair at nicks. Nicks at a target site (black) may be repaired by direct religation 1, which produces no genetic signature; canonical HDR4, 25; or alternative HDR or mutagenic EJ, as shown. A gap is formed by unwinding or nucleolytic processing, shown to occur in the 3′-5′ direction that appears predominant based on results in FIG. 4A. ssDNA partially heterologous to the target (indicated by loops on both strands) anneals to the gap. Heterology is corrected and the donor may be released (left). DNA ends are processed and ligation generates an intact repaired duplex. Note that the donor may be released from a nicked duplex by unwinding, left; or derive from free ssDNA, center; and that a free ssDNA molecule may be the transcribed for correction of heterology, as shown; or it may be incorporated into the gapped molecule for repair subsequent to replication (not shown). Mutagenic EJ may occur if replication forks collide at the processed nick to generate a one-sided DSB (right).

FIGS. 5A-5E demonstrate targets for endonucleolytic cleavage and repair in TL reporters. FIG. 5A depicts diagrams of the TL^{TP} and TL^{NT} reporters, which are nicked by I-AniI nickase on the transcribed and non-transcribed strands (arrowhead) within a GFP gene disabled by two stop codons downstream of the I-AniI site (asterisks). FIG. 5B depicts the sequences of the regions in the TL^{TP} (SEQ ID NO: 208) and TL^{NT} (SEQ ID NO: 209) reporters that includes the ATG translational start site, the I-AniI site (boxed) and two stop codons (dashed boxes underlined). The I-AniI site is flipped in orientation in the TL^{TP} and TL^{NT} reporters. Arrowheads indicate sites of non-transcribed and transcribed strand nicks; the corresponding DSBs have a 4 nt 3′-overhang. The 38 bp insertion in the GFP gene (small letters) that includes the I-AniI site and two nonsense codons is replaced during HDR. Donors carry a 17 nt region heterologous to this insertion (lowercase), flanked by homologous sequence (uppercase). FIG. 5C depicts a diagram of pCS14GFP HDR donor, which carries a defective GFP gene with a deletion of 3′-sequence (X), and shares homology with the TL reporters extending 2.47 kb upstream and 0.56 kb downstream of the 38 bp insertion bearing the I-AniI site. FIG. 5D depicts a diagram of pG-no and pG-An HDR donors, which carry a GFP gene rendered defective by 3′ insertion. pG-no carries two 3′ stop codons (asterisks); pG-An donors carry a single stop codon, and a site for either transcribed (pG-An-TP) or non-transcribed (pG-An-NT) strand nicking by I-AniI (arrowheads), at a position approximately 500 bp downstream of the I-AniI site in the GFP repair target gene. FIG. 5E depicts sequences of the insertions in the pG-no and pG-An HDR donors. pG-no (SEQ ID NO: 210) carries two in-frame stop codons (dashed boxes, underlined); and pG-An-TP (SEQ ID NO: 211) and pG-An-NT (SEQ ID NO: 212) carry an I-AniI site (boxed) and a single in-frame stop codon (dashed boxes, underlined). Homology of the pG donors with the TL reporters extends 0.1 kb upstream and 0.49 kb downstream of the 38 bp insertion in the reporter that includes the I-AniI site. I-AniI sites are boxed, and insertions that interrupt GFP are shown in lower case.

FIG. 6 demonstrates that HDR does not occur upon I-AniI expression in the absence of a repair donor. Representative flow assay of HDR (GFP+) and mutagenic EJ (mCherry+) frequencies following transient transfection of 293T-TL^{TP} and 293T-TL^{NT} cell populations with constructs expressing catalytically inactive I-AniI (I-AniIDEAD) or I-AniI nickase or cleavase, in the absence of repair donor.

FIGS. 7A-7B demonstrates that induction of transcription stimulates HDR at transcribed strand nicks in individual cell lines. Mean HDR (GFP+) frequencies observed following 3 independent transient transfections of 3 different 293-P-Tet-TL^{TP} or 293-P-Tet-TL^{NT} clonal cell lines. The mean and SEM are presented; * and ** indicate p<0.05 and p<0.005, respectively. Pooled data are shown in FIG. 1F.

FIG. 8 demonstrates RAD51 independence of nick-initiated HDR in the HT1080-TL4 cell line. HDR (GFP+) frequencies at nicks or DSBs, using the pCS14GFP dsDNA donor or 99-TOP or 99-BOT ssDNA donors, in the HT1080-TL^{TP}-4 clonal line transiently expressing the RAD51K133R dominant negative mutant. The mean and SEM of 5 or 6 transfections are presented; * and ** indicate p<0.05 and p<0.005, respectively.

FIGS. 9A-9D demonstrate that HDR occurs preferentially at a transcribed strand nick and is stimulated by transcription. FIG. 9A depicts diagrams of the chromosomal I-AniI Traffic Light (TL) reporter (18) that consists of a defective GFP gene containing an I-AniI site and two stop codons (asterisks) near the 5′-end, joined by a T2A translational linker to the mCherry coding sequence in the +2 open reading frame (ORF). The I-AniI site is oriented to nick the transcribed strand in the TL^{TP} reporter and the non-transcribed strand in the TL^{NT} reporter, as indicated by the two arrowheads. GFP+ cells will result from HDR of the chromosomal reporter using an exogenous donor; while mCherry+ cells will result from cleavage at the I-AniI site followed by mutEJ that puts mCherry in the +2 reading frame. FIG. 9B depicts graphs of HDR (GFP+) and mutEJ (mCherry+) frequencies calculated from independent transient transfections of 293T-TL^{TP} or 293T-TL^{NT} cells (I-AniIDEAD; n=2; Nick, n=4; DSB, n=4), one example of which is shown in FIG. 9B. Mean and standard error of the mean (SEM) are shown. Differences in HDR between the two TL^{TP} and TL^{NT} cells are significant at nicks (** indicates p<0.005) but not at DSBs (p=0.23). FIG. 9C depicts graphs of HDR (GFP+) frequencies, based on pooled results from a total of 11 independent transfections of three different clonal 293-P-Tet-TL^{TP} or 293-P-Tet-TL^{NT} cell lines carried out in the absence (OFF) or presence (ON) of 1 μg/ml doxycycline, and normalized to frequencies for cells cultured without inducer. Cell lines were analyzed individually and frequencies for HDR at nicks or DSBs in each were normalized to frequencies for cells cultured without doxycycline (data not shown). FIG. 9D depicts a model diagramming how transcription may affect repair at a transcribed strand nick (TL^{TP}) by unwinding DNA to expose the recombinogenic 3′ end; but inhibit repair of a non-transcribed strand (TL^{NT}) by occluding the 3′ end and exposing the less recombinogenic 5′ end.

FIGS. 10A-10C demonstrate donor use and donor strand bias in HDR at nicks. FIG. 10A depicts diagrams of TL^{TP} and TL^{NT} reporters, with 99 nt ssDNA oligonucleotide donors complementary to the nicked (cN) or intact (cI) strand of each shown below. Donors were centered at the nick and complementary to the indicated strand except for a 17 nt region of heterology, containing GFP coding sequence which replaces the stop codons and I-AniI site in the reporter to enable GFP expression. FIG. 10B depicts grpahs of HDR (GFP+) frequencies at nicks or DSBs, using ssDNA donors complementary to the nicked (cN) or intact (cI) DNA strand, in 293T-TL7^{TP} and HT1080 TL4^{TP} clonal lines; mean and SEM calculated from at least 5 transfections; ** p<0.005. FIG. 10C depicts graphs of HDR (GFP+) frequencies at nicks or DSBs, using ssDNA donors complementary to the nicked (cN) or intact (cI) DNA strand, in 293T-TL^{NT} and 293T-TL^{TP} cell populations. Mean and SEM calculated from at least 5 transfections; * and ** indicate p<0.05 and p<0.005, respectively.

FIGS. 11A-11D demonstrate that downregulation of canonical HDR stimulates HDR at nicks by ssDNA or nicked dsDNA donors. FIG. 11A depicts graphs of HDR (GFP+) frequencies at nicks or DSBs in the 293T-TL7^{TP} clonal line treated with siRAD51, using the pCS14GFP dsDNA donor (n=4) or cN (n=2) or cI (n=4) ssDNA donors, as indicated. Mean and SEM are presented; * and ** indicate p<0.05 and p<0.005, respectively. FIG. 11B depicts graphs of HDR (GFP+) frequencies at nicks in 293T-TL^{NT} and 293T-TL^{TP} cell populations transiently expressing RAD51K133R, using indicated donors (n=4-6). FIG. 11C depicts graphs of HDR (GFP+) frequencies at nicks with the cI ssDNA donor or at DSBs with the dsDNA donor in the 293T-TL7^{TP} clonal line treated with the indicated siRNA (n=6-12). FIG. 11D depicts, on the left, a diagram of dsDNA donors, with no I-AniI site (pG-no) or carrying an I-AniI site oriented for intracellular nicking of the transcribed (pGAn-TP) or non-transcribed (pGAn-NT) strand. The I-AniI site in pGAn-TP and pGAn-NT is approximately 500 bp downstream of the I-AniI site in the reporter GFP gene targeted for repair. The donors contain 100 bp of upstream and 500 bp of downstream homology with the chromosomal reporter and the promoters are not homologous to the TL promoter (FIGS. 15A-15C). On the right is a graph of HDR (GFP+) frequencies at nicks in the 293T-TL7^{TP} clonal line transiently expressing the RAD51K133R dominant negative mutant with intact or nicked dsDNA donors (n=5); ** p<0.005.

FIGS. 12A-12D demonstrate that CRISPR/Cas9 generated nicks initiate alternative HDR and are associated with less mutEJ than are DSBs. FIG. 12A depicts sequence of the portion of the TL^{TP} reporter (SEQ ID NO: 213) containing CRISPR/Cas9 and I-AniI target sites (open and closed arrowheads). The insertion (lower case) bearing the I-AniI site and stop codons (underlined) and a portion of the GFP coding sequence (upper case) are shown. The CRISPR guide RNA and PAM sequence are indicated. FIG. 12B depicts graphs of HDR (GFP+) frequencies at nicks or DSBs in the 293T-TL7^{TP} clonal line following transient transfection of a Cas9D10A (Nick) or Cas9 (DSB) expression plasmid, the guide RNA expression plasmid and either the dsDNA plasmid donor pCS14GFP or cI and cN ssDNA donors, as indicated. Mean and SEM calculated from 3 transfections; ** indicates p<0.005. FIG. 12C depicts graphs of MutEJ (mCherry+) frequencies at nicks or DSBs in the 293T-TL7^{TP} clonal line; same cells as in FIG. 12B. FIG. 12D depicts graphs of the ratio of HDR to mutEJ (GFP+:mCherry+ cells) compiled from transfections of 293T-TL7^{TP} in FIG. 12B, analyzing HDR at template-strand nicks using the cI ssDNA donor in siBRCA2-treated cells; and HDR at DSBs using a dsDNA donor in untreated cells.

FIG. 13 demonstrates a relation between ssDNA donor homology and HDR at nicks. 293T-TL^{TP} (left) and 293T-TL^{NT} (right) cell populations, transiently expressing the I-AniI nickase and RAD51K133R, were provided with a 75 nt ssDNA donor centered at the nick or extending either 3′ or 5′ of the nick. ssDNA donors were complementary to the nicked (cN) or intact (cI) strands and carried a 17 nt region of heterology (dotted line), and homologous flanking sequences of indicated lengths. Graphs show HDR frequencies for donors extending either 3′ (light gray bar) or 5′ (dark gray bar) normalized to the donor centered at the nick (white bar), and represent mean and SEM of at least 7 transfections.

FIG. 14 depicts a working model for pathways of HDR at nicks. Left, RAD51-dependent HDR using a dsDNA donor. A gap is exposed at the nicked target, and BRCA2 loads RAD51 on the free 3′ end, enabling invasion of a homologous dsDNA donor, as in canonical DSB repair. Right, RAD51/BRCA2-independent HDR. A gap is exposed at the nicked target, and the donor anneals to either the nicked (left) or intact (right) strand of the duplex, independent of RAD51/BRCA2. Heterology (white boxes) and repair synthesis (dashed line) are shown. Arrowheads represent nucleolytic removal of DNA, either by excision or flap cleavage.

FIGS. 15A-15C demonstrate targets for endonucleolytic cleavage and repair in TL reporters. FIG. 15A depicts sequences of the regions in the TL^{TP} (SEQ ID NO: 208) and TL^{NT} (SEQ ID NO: 209) reporters that include the ATG translational start site, the I-AniI site (boxed) and two stop codons (underlined). The I-AniI site is flipped in orientation in the TL^{TP} and TL^{NT} reporters. Arrowheads indicate sites of non-transcribed and transcribed strand nicks; the corresponding DSBs have a 4 nt 3′-overhang. The 38 bp insertion in the GFP gene (small letters) that includes the I-AniI site and two nonsense codons is replaced during HDR. Donors carry a 17 nt region heterologous to this insertion (lowercase), flanked by homologous sequence (uppercase). FIG. 15B depicts diagrams of the TL^{TP} and TL^{NT} reporters, which are nicked by I-AniI nickase on the transcribed and non-transcribed strands (arrowhead) within a GFP gene disabled by two stop codons at the 5′-end, just downstream of the I-AniI site (asterisks). The pCS14GFP HDR donor carries a defective GFP gene with a deletion of 3′-sequence (X), and shares extensive homology with the TL reporters extending 2.5 kb upstream and 0.6 kb downstream of the 38 bp insertion bearing the I-AniI site. The pG-no and pG-An HDR donors carry a GFP gene rendered defective by 3′ insertion and share homology with the TL reporters extending 0.1 kb upstream and 0.6 kb downstream of the 38 bp insertion bearing the I-AniI site. pG-no carries two premature stop codons (asterisks); pG-An donors carry a single premature stop codon, and a site for either transcribed (pG-An-TP) or non-transcribed (pG-An-NT) strand nicking by I-AniI (arrowheads), at a position approximately 500 bp downstream of the I-AniI site in the GFP repair target gene. Homology of the pG donors with the TL reporters extends 0.1 kb upstream and 0.49 kb downstream of the 38 bp insertion in the reporter that includes the I-AniI site. FIG. 15C depicts sequences of the 3′ end of the GFP coding sequence from pEGFP-N1 (SEQ ID NO: 214) and the pG-no (SEQ ID NO: 215), pG-An-TP (SEQ ID NO: 216) and pG-An-NT (SEQ ID NO: 217) HDR donors. pG-no carries two premature stop codons; and pG-An-TP and pG-An-NT carry an I-AniI site and a single premature stop codon. The I-AniI sites are boxed, and stop codons are underlined.

FIGS. 16A-16B demonstrate that I-AniI nuclease activity and a repair donor are required to generate GFP+ cells. FIG. 16A depicts a representative flow cytometric assay of HDR (GFP+) and mutEJ (mCherry+) cells generated following transient transfection of 293T-TL^{TP} and 293T-TL^{NT} cell populations with constructs expressing catalytically inactive I-AniI (I-AniIDEAD) or I-AniI nickase (Nick) or cleavase (DSB), and the dsDNA plasmid donor pCS14GFP. FIG. 16B depicts a representative flow assay of HDR (GFP+) and mutEJ (mCherry+) cells generated following transient transfection of 293T-TL^{TP} and 293T-TL^{NT} cell populations with constructs expressing catalytically inactive I-AniI (I-AniIDEAD) or I-AniI nickase or cleavase, in the absence of repair donor.

FIGS. 17A-17C demonstrate that nick-iniated mutEJ is transcription-independent and occurs less frequently than DSB-initiated mutEJ. FIG. 17A depicts graphs of MutEJ (mCherry+) frequencies at nicks or DSBs in 293T-TL^{TP} and 293T-TL^{NT} cell populations expressing I-AniI nickase or cleavase; and the ratio of HDR:mutEJ (GFP+:mCherry+ cells). Mean and SEM calculated from 4 transfections; * and ** indicate p<0.05 and p<0.005, respectively. FIG. 17B depicts graphs of MutEJ (mCherry+) frequencies at nicks or DSBs in 293-P-Tet-TL^{TP} or 293-P-Tet-TL^{NT} cell lines cultured in the presence or absence of doxycycline; and normalized to frequencies for cells cultured without doxycycline (n=11). FIG. 17C depicts graphs of MutEJ (mCherry+) frequencies at (left) nicks or (middle) DSBs in the 293T-TL^{TP}-7 clonal line transiently expressing the RAD51K133R dominant negative mutant, using the dsDNA donor or the cN or cI ssDNA donors (n=3-6). The ratio (right) of HDR to mutagenic EJ (GFP+:mCherry+ cells) was compiled from transfections carried out under optimal HDR conditions for nicks (transcribed-strand nick, bottom strand donor, RAD51 inhibited) or DSBs (dsDNA donor).

FIGS. 18A-18B demonstrate that downregulation of RAD51 or BRCA2 stimulates HDR at nicks using ssDNA donors. FIG. 18A depicts graphs of HDR (GFP+) frequencies at nicks or DSBs, using the pCS14GFP dsDNA donor or cN or cI ssDNA donors, in the 293T-TL^{TP}-7 clonal line transiently expressing the RAD51K133R dominant negative mutant, (n=3-6). The mean and SEM of 5 or 6 transfections are presented; * and ** indicate p<0.05 and p<0.005, respectively. FIG. 18B depicts graphs of HDR (GFP+) frequencies at nicks or DSBs, using the indicated donors, in the HT1080-TL4^{TP} clonal line transiently expressing the RAD51K133R dominant negative mutant (n=5-6).

FIG. 19 depicts a working model for HDR using a ssDNA donor complementary to the intact strand (cI). A gap is exposed at the nicked target, shown to occur by a 3′-5′ helicase (unwinding, slanted line) and 5′-3′ nucleolytic resection (resection, dotted line), although this example is not meant to exclude the potential contribution of 5′-3′ unwinding or 3′-5′ excision to gap formation. The ssDNA donor complementary to the intact strand (cI; homology, black; heterology, white boxes) anneals to the gap, independent of RAD51/BRCA2. The donor can then be incorporated into the target and the heterology eliminated by mismatch repair (left); or, if DNA replication and mitosis occur prior to mismatch repair (MMR), heterology may instead be resolved by segregation (not shown). Alternatively, the donor may not be incorporated but direct mismatch repair and then be released by strand displacement accompanying repair synthesis, followed by ligation to generate an intact duplex at the target (right).

FIG. 20 depicts a diagram of a nicked dsDNA donor promoting HDR by acting as a template for repair synthesis. 1. Nicked target and nicked donor. Nicks are separated by 500 bp, and are shown on either the opposite (left) or same (right) strand as the donor nick; homology black, heterology, white boxes. 2. Unwinding occurs at the nicks in both the target and donor. If the donor and target nicks are on opposite strands (left) then the free 3′ end of the target strand anneals to the free 3′ end of the donor. If the donor and target nicks are on identical strands (right) then the free 3′ end of the target strand anneals to the intact strand of the donor. 3. The donor is used as a template for repair synthesis (dotted lines) primed by that 3′ end of the target. 4. Newly synthesized region is released from the donor template, then reanneals to the target, displacing a region carrying a 5′ flap, which is cleaved by endonucleases (arrowheads). 5. Following flap removal, ligation completes HDR. The mechanism of donor unwinding is unknown but is shown in the direction required to traverse the 500 bp between the regions of the nick in the target and donor (left, 3′-5′; right, 5′-3′); the experiments in FIG. 11D used a nicked plasmid (circular) donor so that unwinding of 4200 bp in the direction opposite to that shown might also allow productive annealing.

FIG. 21 depicts a diagram of a nicked dsDNA donor strand promoting HDR by directing mismatch repair. 1. Nicked target and nicked donor. Nicks are separated by 500 bp, and are shown on either the opposite (left) or same (right) strand as the donor nick; homology black, heterology, white boxes. 2. Unwinding initiates at the nicks in both the target and donor. If the donor and target nicks are on opposite strands (left) then the intact donor and target strands anneal. If the donor and target nicks are on the same strands (right), then the free 3′ end of the donor anneals to the exposed gap on the target. 3. The donor directs mismatch repair (MMR). 4. The 3′ end of the target nick primes DNA synthesis (dotted line). This may require or be accompanied by flap removal (arrowheads). 5. The donor is released from the target, and the target undergoes nucleolytic processing (arrowhead). 6. Following flap removal, ligation completes HDR. The mechanism of donor unwinding is unknown but is shown in the direction required to traverse the 500 bp between the regions of the nick in the target and donor (left, 5′-3′; right, 3′-5′); the experiments in FIG. 11D used a nicked plasmid (circular) donor so that unwinding of 4200 bp in the direction opposite to that shown might also allow productive annealing.

FIG. 22 depicts expression analysis of siRNA treated 293T-TL7^{TP} cells. cDNAs were synthesized at 48 hrs post siRNA transfection and used as template for PCR by primers directed against the indicated genes. Band intensities are given relative to the siNT2 band for each primer pair.

FIG. 23 depicts fractions of GFP+, mCherry+ and BFP+ cells among total live cells, and fractions of GFP+ and mCherry+ among BFP+ cells for each relevant figure panel. Mean and standard error of the mean (SEM) are shown below each data set. Two-tailed t-tests were used to determine the p-values that are displayed to the right of the raw data. For FIG. 13, the mean and SEM were converted to values relative to the raw values for the centered oligonucleotides.

FIG. 24 depicts graphs of the frequency of HDR at nicks using an ssDNA donor when the expression of the listed genes is inhibited.

FIG. 25 depicts a graph of the ratio of HDR to mutEJ at nicks when the expression of the listed genes is inhibited.

FIG. 26 depicts an exemplary embodiment of a “tagging” use of the methods described herein. The tagging of endogenous RECQL5 using alternative HDR is depicted. On top is a diagram of the 3′ end of genomic RECQL5-HA. Primer F3 is specific to the HA tagged locus. On the bottom nested PCT of genomic DNA from H1080 cells transfected with the indicated ssDNA donors and contructs expressing Cas9-D10A nickase, dominant negative RAD51, and either no gRNA, RECQL5-gRNA1 or RECQL5-gRNA2. First round of PCR: 25 cycles with primers F1 and R1 (not shown). Second round of PCR used either primers F2 and R2 (left) or F3 and R2 (right).

### DETAILED DESCRIPTION

DNA damage repair is a complex set of processes that detect and attempt to reverse damage and errors in the DNA sequence that arise from numerous causes. One type of damage is the physical interruption of the backbone (e.g. a cut or severing) of one or more strands of DNA, typically referred to as a “nick” if only one strand of a dsDNA molecule is severed and as a “double-strand break” (DSB) if both strands of a dsDNA molecule are severed. It is noted that for a break to occur, both strands must be broken at approximately the same location. While a DSB can be blunt ended or have either a 5′ or 3′ overhang, if the strands are each cleaved too far apart, the overhangs will continue to anneal to each other and exist as two nicks, not one DSB. The degree of proximity of the cleavage sites necessary to generate a break will vary, e.g. with the % G/C content of the sequences and the temperature and/or salt concentration. In some embodiments, the cleavage sites must be less than 10 nt from each other to generate a DSB, e.g., less than 10 nt, less than 9 nt, less than 8 nt, less than 7 nt, less than 6 nt, less than 5 nt, less than 4 nt, less than 3 nt, less than 2 nt, or at the same location.

One type of repair that cells use to address nicks and breaks is homology-directed repair (HDR). As used herein, “homology-directed repair” or “HDR” refers to the specialized form of DNA repair that takes place, for example, during repair of double-strand breaks in cells. This process requires nucleotide sequence homology, uses a “donor” molecule to template repair of a “target” molecule (i.e., the one that experienced the double-strand break), and can lead to the transfer of genetic information from the donor to the target. HDR can result in an alteration of the sequence of the target molecule (e.g., insertion, deletion, mutation), if the donor nucleic acid molecule differs from the target molecule and part or all of the sequence of the donor molecule is incorporated into the target DNA. In some embodiments, the process can repair a nick.

As described herein, the inventors have discovered that there are in fact two separate HDR mechanisms; canonical HDR and alternative HDR. Canonical HDR has been studied at DSBs and requires BRCA2 and RAD51, and typically employs a dsDNA donor molecule. In contrast, “alternative HDR” is an HDR mechanism that is suppressed by BRCA2, RAD51, and functionally-related genes (see, e.g. Table 1). Alternative HDR uses a ssDNA or nicked dsDNA donor molecule. In some embodiments, alternative HDR is positively regulated by BRCA1 and/or requires BRCA1. Alternative HDR is demonstrated herein to have a lower error rate than canonical HDR. Accordingly, provided herein are methods for inducing or suppressing alternative HDR, e.g. to permit improved genetic modification or to reduce genomic instability, e.g. in a cancer.

In some embodiments, canonical HDR has an error rate at least about 2× that of alternative HDR. In some embodiments, canonical HDR has an error rate at least about 3× that of alternative HDR. In some embodiments, canonical HDR has an error rate at least about 4× that of alternative HDR. In some embodiments, canonical HDR has an error rate at least about 5× that of alternative HDR.

In one aspect, described herein is a method of increasing alternative homology-directed repair (alternative HDR) at a target nucleic acid (e.g. DNA) nick, the method comprising contacting the nucleic acid with an inhibitor of a gene expression product of a gene selected from Table 1. In some embodiments, a cell-free system comprises the target nucleic acid molecule, e.g. the target nucleic acid molecule is present in a cell extract or a subcellular fraction. In one aspect, described herein is a method of increasing alternative homology-directed repair (alternative HDR) at a nucleic acid (e.g. DNA) nick in a cell, the method comprising contacting the cell with an inhibitor of a gene expression product of a gene selected from Table 1. In some embodiments, the inhibitor is an inhibitor of RAD51; BRCA2; PALB2 and/or SHFM1. Increasing alternative HDR can refer to, e.g. increasing the speed with which a particular nick or break is repaired by alternative HDR, and/or increasing the percentage of nicks or breaks repaired by alternative HDR instead of other damage repair pathways (e.g. canonical HDR). The methods can also increase the efficiency of targeted gene modification, as discussed herein.

As demonstrated herein, inhibition of BRCA2-related activity increases alternative HDR. The genes of Table 1 promote BRCA2 activity. It is therefore specifically contemplated herein that inhibition of one or more of these genes increases alternative HDR. In some embodiments, the methods described herein can relate to the inhibition (or in some aspects, activation) of one or more genes selected from Table 1, e.g. one gene, two genes, three genes, four genes, or any number of genes selected from Table 1 as described herein.

In some embodiments, the methods described herein relate to inhibition (or in some aspects, activation) of one or more of RAD51; BRCA2; PALB2 and/or SHFM1. In some embodiments, the methods described herein relate to inhibition (or in some aspects, activation) of RAD51. In some embodiments, the methods described herein relate to inhibition (or in some aspects, activation) of BRCA2. In some embodiments, the methods described herein relate to inhibition (or in some aspects, activation) of PALB2. In some embodiments, the methods described herein relate to inhibition (or in some aspects, activation) of SHFM1. In some embodiments, the methods described herein relate to inhibition (or in some aspects, activation) of RAD51 and BRCA2. In some embodiments, the methods described herein relate to inhibition (or in some aspects, activation) of RAD51 and PALB2. In some embodiments, the methods described herein relate to inhibition (or in some aspects, activation) of RAD51 and SHFM1. In some embodiments, the methods described herein relate to inhibition (or in some aspects, activation) of BRCA2 and PALB2. In some embodiments, the methods described herein relate to inhibition (or in some aspects, activation) of BRCA2 and SHFM1. In some embodiments, the methods described herein relate to inhibition (or in some aspects, activation) of PALB2 and SHFM1. In some embodiments, the methods described herein relate to inhibition (or in some aspects, activation) of RAD51, BRCA2, and PALB2. In some embodiments, the methods described herein relate to inhibition (or in some aspects, activation) of RAD51, BRCA2, and SHFM1. In some embodiments, the methods described herein relate to inhibition (or in some aspects, activation) of RAD51, PALB2, and SHFM1. In some embodiments, the methods described herein relate to inhibition (or in some aspects, activation) of BRCA2, PALB2, and SHFM1. In some embodiments, the methods described herein relate to inhibition (or in some aspects, activation) of RAD51, PALB2, SHFM1, and BRAC2.

As used herein, the term “RAD51” refers to a protein that forms a helical nucleoprotein filament on DNA and controls the homology search and strand pairing of DNA damage repair. Sequences for RAD51 polypeptides and nucleic acids encoding them for a number of species are known in the art, e.g. human RAD51 (NCBI Gene ID: 5888) polypeptide (SEQ ID NO: 144; NCBI Ref Seq: NP_{—}001157741) and nucleic acid (SEQ ID NO: 058; NCBI Ref Seq: NM_{—}001164269).

As used herein, the term “BRCA2” refers to a tumor suppressor gene product that normally functions by binding single-stranded DNA at DNA damage sites and interacting with RAD51 to promote strand invasion. Sequences for BRCA2 polypeptides and nucleic acids encoding them for a number of species are known in the art, e.g. human BRCA2 (NCBI Gene ID: 675) polypeptide (SEQ ID NO: 095; NCBI Ref Seq: NP_{—}000050) and nucleic acid (SEQ ID NO: 009; NCBI Ref Seq: NM_{—}000059).

As used herein, the term “SHFM1” refers to a 26S proteasome complex subunit that interacts directly with BRCA2. Sequences for SHFM1 polypeptides and nucleic acids encoding them for a number of species are known in the art, e.g. human SHFM1 (NCBI Gene ID: 7979) polypeptide (SEQ ID NO: 152; NCBI Ref Seq: NP_{—}006295) and nucleic acid (SEQ ID NO: 066; NCBI Ref Seq: NM_{—}006304).

As used herein, the term “PALB2” refers to a DNA-binding protein that binds to single-strand DNA and facilitates accumulation of BRCA2 at the site of DNA damage. PALB2 also interacts with RAD51 to promote strand invasion. Sequences for PALB2 polypeptides and nucleic acids encoding them for a number of species are known in the art, e.g. human PALB2 (NCBI Gene ID: 79728) polypeptide (SEQ ID NO: 132; NCBI Ref Seq: NP_{—}078951) and nucleic acid (SEQ ID NO: 046; NCBI Ref Seq: NM 024675).

TABLE 1
Genes/Gene Product the Promote BRCA2 Activity

| | NCBI | mRNA | |
|---|---|---|---|
| | Gene ID | SEQ ID | Polypeptide |
| Gene Name | NO: | NO: | SEQ ID NO: |
| ABL1 | 25 | 001 | 087 |
| ATM | 472 | 002 | 088 |
| ATR | 545 | 003 | 089 |
| AURKB | 9212 | 004 | 090 |
| BACH1 | 571 | 005 | 091 |
| BARD1 | 580 | 006 | 092 |
| BCCIP | 56647 | 007 | 093 |
| BLM | 641 | 008 | 094 |
| BRCA2 | 675 | 009 | 095 |
| BRCC3 | 79184 | 010 | 096 |
| BRE | 9577 | 011 | 097 |
| BUB1B | 701 | 012 | 098 |
| C11orf30 | 56946 | 013 | 099 |
| CCNA2 | 890 | 014 | 0100 |
| CDC45 | 8318 | 015 | 0101 |
| CDK1 | 983 | 016 | 0102 |
| CDK2 | 1017 | 017 | 0103 |
| CDK4 | 1019 | 018 | 0104 |
| CHEK1 | 1111 | 019 | 0105 |
| CHEK2 | 11200 | 020 | 0106 |
| DMC1 | 11144 | 021 | 0107 |
| ECD | 11319 | 022 | 0108 |
| FANCD2 | 2177 | 023 | 0109 |
| FANCE | 2178 | 024 | 0110 |
| FANCG | 2189 | 025 | 0111 |
| FANCI | 55215 | 026 | 0112 |
| FLNA | 2316 | 027 | 0113 |
| FYN | 2534 | 028 | 0114 |
| GRB2 | 2885 | 029 | 0115 |
| H2AFX | 3014 | 030 | 0116 |
| HDAC1 | 3065 | 031 | 0117 |
| HDAC2 | 3066 | 032 | 0118 |
| HMG20B | 10362 | 033 | 0119 |
| KAT2B | 8850 | 034 | 0120 |
| KIF4A | 24137 | 035 | 0121 |
| LMNA | 4000 | 036 | 0122 |
| MCPH1 | 79648 | 037 | 0123 |
| MGMT | 4255 | 038 | 0124 |
| MLH1 | 4292 | 039 | 0125 |
| MLH3 | 27030 | 040 | 0126 |
| MND1 | 84057 | 041 | 0127 |
| MORF4L1 | 10933 | 042 | 0128 |
| MRE11A | 4361 | 043 | 0129 |
| MSH4 | 4438 | 044 | 0130 |
| MTA2 | 9219 | 045 | 0131 |
| PALB2 | 79728 | 046 | 0132 |
| PCNA | 5111 | 047 | 0133 |
| PDS5B | 23047 | 048 | 0134 |
| PLK1 | 5347 | 049 | 0135 |
| PMS1 | 5378 | 050 | 0136 |
| PMS2 | 5395 | 051 | 0137 |
| PSMC3IP | 29893 | 052 | 0138 |
| PSMD3 | 5709 | 053 | 0139 |
| PSMD6 | 9861 | 054 | 0140 |
| RAD21 | 5885 | 055 | 0141 |
| RAD23A | 5886 | 056 | 0142 |
| RAD50 | 10111 | 057 | 0143 |
| RAD51 | 5888 | 058 | 0144 |
| RAD51B | 5890 | 059 | 0145 |
| RAD51C | 5889 | 060 | 0146 |
| RBBP8 | 5932 | 061 | 0147 |
| RPA1 | 6117 | 062 | 0148 |
| RPA2 | 6118 | 063 | 0149 |
| RPA3 | 6119 | 064 | 0150 |
| SERPINH1 | 871 | 065 | 0151 |
| SHFM1 | 7979 | 066 | 0152 |
| SIRT1 | 23411 | 067 | 0153 |
| SIRT2 | 22933 | 068 | 0154 |
| SKP2 | 6502 | 069 | 0155 |
| SMAD1 | 4086 | 070 | 0156 |
| SMAD2 | 4087 | 071 | 0157 |
| SMAD3 | 4088 | 072 | 0158 |
| SMC3 | 9126 | 073 | 0159 |
| SP1 | 6667 | 074 | 0160 |
| SPO11 | 23626 | 075 | 0161 |
| STAT5A | 6776 | 076 | 0162 |
| SYCP3 | 50511 | 077 | 0163 |
| TEX15 | 56154 | 078 | 0164 |
| TOP3A | 7156 | 079 | 0165 |
| TP53 | 7157 | 080 | 0166 |
| UBC | 7316 | 081 | 0167 |
| UQCC1 | 55245 | 082 | 0168 |
| USP11 | 8237 | 083 | 0169 |
| WDR16 | 146845 | 084 | 0170 |
| XRCC3 | 7517 | 085 | 0171 |

The gene names listed in Table 1 are common names. The sequences and NCBI Gene ID numbers provided for each gene listed in Table 1 are the human sequences and accessions. Homologous genes from other species may be readily identified, e.g. the identified homologs in the NCBI database, or by querying databases, e.g. via BLAST.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, BRCA2, e.g. its ability to decrease the level and/or activity of BRCA2, can be determined, e.g. by measuring the level of an expression product of BRCA2 and/or the activity of BRCA2 (e.g. the ability of BRCA2 to suppress alternative HDR). Methods for measuring the level of a given mRNA and/or polypeptide are known to one of skill in the art, e.g. RTPCR can be used to determine the level of RNA and Western blotting with an antibody (e.g. an anti-BRCA2 antibody, e.g. Cat No. ab97; Abcam; Cambridge, Mass.) can be used to determine the level of a polypeptide. The activity of, e.g. BRCA2 can be determined using methods known in the art and described in the Examples herein (e.g. assays to measure the rate of alternative HDR). In some embodiments, the inhibitor can be an inhibitory nucleic acid; an aptamer; an antibody reagent; an antibody; or a small molecule. In some embodiments, the inhibitor of a target can be an inhibitor specific for that target (e.g. a RAD51-specific inhibitor, a BRCA2-specific inhibitor, a PALB2-specific inhibitor, and/or a SHFM1-specific inhibitor). An inhibitor specific for a given target is an inhibitor which binds specifically to the target molecule (e.g. a mRNA or a polypeptide).

In some embodiments, an inhibitor will directly bind to the targeted factor, e.g. BRCA2 or to its mRNA. In some embodiments, an inhibitor will directly result in the cleavage of the targeted factor's mRNA, e.g., via RNA interference. In some embodiments, an inhibitor can act in a competitive manner to inhibit activity of the targeted factor. In some embodiments, an inhibitor can comprise a portion of the target factor and act as a competitive or dominant negative factor for interactions normally involving the targeted factor.

In some embodiments, the methods described herein can comprise contacting the cell with two or more inhibitors, e.g. two inhibitors, three inhibitors, four inhibitors, or more inhibitors. In some embodiments, the methods described herein can comprise contacting the cell with a plurality of inhibitors, e.g. an inhibitor of RAD51 and an inhibitor of BRCA2. In some embodiments, an inhibitor can inhibit multiple targets, e.g. an antibody reagent with bispecificity. In some embodiments, multiple types of inhibitors can be used, e.g. an antibody reagent specific for BRCA2 and a small molecule inhibitor of RAD51.

In some embodiments, an inhibitor of a gene expression product of a gene of Table 1 can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part of the targeted mRNA transcript. The use of these iRNAs permits the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a gene selected from Table 1. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.), 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA as described herein involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, an inhibitor of a gene expression product of a gene of Table 1 can be an antibody reagent specific for the respective polypeptide. For example, in some embodiments, a BRCA2 inhibitor can be an anti-BRCA2 antibody reagent. Antibodies have, historically, been viewed as unable to cross the plasma membrane. However, antibodies have been demonstrated to gain access to intracellular protein targets (see, e.g. Guo et al., Science Translational Med. 2011 3:99ra85; WO2008/136774; Guo et al. Cancer Biol and Ther 2008 7:752-9; and Ferrone. Sci Transl Med 2011 3:99ps38) both in cultured cells and in vivo.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to the target polypeptide.

In some embodiments, an inhibitor of a gene expression product of a gene of Table 1 can be a small molecule. Small molecule inhibitors of the various targets described herein are known in the art. For example, inhibitors of RAD51 can include but are not limited to IBR2; RI-1; RI-2; and B02.

Because of the lower mutagenesis rate associated with alternative HDR, promotion of alternative HDR during gene modification (e.g. genetic engineering) can reduce the rate of unwanted mutations introduced during modification. In one aspect, provided herein is a method of gene modification, the method comprising contacting a cell with: a) a donor nucleic acid molecule comprising the gene modification to be made in the cell; b) a nickase; and c) an inhibitor of one or more genes of Table 1. In some embodiments, the inhibitor is an inhibitor of RAD51; BRCA2; PALB2 and/or SHFM1.

As used herein, “gene modification” refers to a process of introducing a desired modification into a nucleic acid sequence. In some embodiments, gene modification can be targeted gene modification, i.e. the introduction of a desired modification at a particular genetic locus. Modifications can include insertions, deletions, and/or mutations relative to the original sequence. In the aspects described herein, the modification can be comprised by a donor nucleic acid molecule and the modification can be introduced into a target nucleic acid sequence via the methods described herein.

In some embodiments, a method of gene modification can comprise introducing a detectable “tag” to an existing or endogenous gene present in the target nucleic acid. Because of the lower rates of mutagenesis caused by the methods described herein, it is particularly well suited to such modifications. Detectable tags are nucleic acid sequences which generate or comprise the ability to generate a detectable signal (e.g. by catalyzing a reaction converting a compound to a detectable product) either as a transcribed nucleic acid product or as a translated polypeptide product. Detectable tags can include, by way of non-limiting example, e.g., fluorescent polypeptides (e.g. GFP; mCherry; CFP; GFP; ZsGreen1; YFP; ZsYellow1; mBanana; mOrange; DsRed; tdTomato; DsRed2; mStrawberry; HcRed1; mRaspberry; E2-Crimson; mPlum; Dendra 2; Timer; PAmCherry; and Cerulean fluorescent protein), epitope tags (e.g. HA, FLAG, V5, VSV-G, HSV, biotin, and Myc), or TRX.

As used herein, “donor nucleic acid molecule” refers to a nucleic acid molecule which has been selected and introduced (e.g. introduced into a cell) to serve as a template for alternative HDR repair. In some embodiments, a donor nucleic acid molecule can comprise a modification to be introduced into the target cell, e.g. at a nick or break. In some embodiments, a donor nucleic acid molecule can be single-stranded or double-stranded. In some embodiments, donor nucleic acid molecule can comprise, e.g., DNA, RNA, or modified versions thereof, e.g. LNA. In some embodiments, the donor nucleic acid molecule can be ssDNA. In some embodiments, a donor nucleic acid molecule can be a nicked dsDNA.

In some embodiments, the donor nucleic acid molecule can be at least about 20 nt in length, e.g. at least about 20 nt in length, at least about 25 nt in length, at least about 30 nt in length, at least about 40 nt in length, at least about 50 nt in length, at least about 60 nt in length, at least about 70 nt in length, at least about 100 nt in length, at least about 200 nt in length, at least about 300 nt in length, at least about 400 nt in length, at least about 500 nt in length, at least about 1 kb in length, at least about 2 kb in length, at least about 3 kb in length, at least about 4 kb in length, or at least about 5 kb in length. In some embodiments, the donor nucleic acid molecule can be from about 20 nt to about 1000 nt in length. In some embodiments, the donor nucleic acid molecule can be from about 20 nt to about 500 nt in length. In some embodiments, the donor nucleic acid molecule can be from about 50 nt to about 200 nt in length.

In some embodiments, the donor nucleic acid molecule can comprise a portion complementary to the strand of the target nucleic acid molecule that is not nicked by the nickase. In some embodiments, a portion of the donor nucleic acid molecule can specifically hybridize to the strand of the target nucleic acid molecule that is not nicked by the nickase, e.g. under the conditions under which the modification process will occur. In some embodiments, the complementary portion is at least 20 nucleotides in length, e.g., 20 nt or longer, 25 nt or longer, 30 nt or longer, 40 nt or longer, or 50 nt or longer. In some embodiments, the donor nucleic acid molecule can comprise multiple portions complementary to the strand of the target nucleic acid molecule that is not nicked by the nickase, e.g. 2 portions, 3 portions, or 4 or more portions. In some embodiments, the multiple complementary portions can flank a modification, e.g. an insertion or deletion comprised by the donor nucleic acid molecule can be flanked by portions of the nucleic acid molecule that are complementary to the strand of the target nucleic acid molecule that is not nicked by the nickase.

In some embodiments, the efficiency of gene modification can be increased if the portion of the donor nucleic acid molecule which anneals to the target nucleic acid molecule is centered around the location of the nick generated in the target nucleic acid molecule. In some embodiments, the portion of the donor nucleic acid molecule that is complementary to a strand of the target nucleic acid molecule is substantially centered with respect to the location of the nick. In some embodiments, a molecule can be substantially centered if no more than 70% of the molecule is located to either side of the reference point (e.g. the location of the nick), e.g. 70% or less, 65% or less, 60% or less, 55% or less, or about 50% of the molecule is located to either side of the reference point. In some embodiments, a portion of a molecule can be substantially centered if no more than 70% of the portion of the molecule is located to either side of the reference point (e.g. the location of the nick), e.g. 70% or less, 65% or less, 60% or less, 55% or less, or about 50% of the portion of the molecule is located to either side of the reference point.

As used herein, “nuclease” refers to an enzyme capable of cleaving the phosphodiester bonds between the nucleotide subunits of nucleic acids. Nucleases can be site-specific, i.e. site-specific nucleases cleave DNA bonds only after specifically binding to a particular sequence. Therefore, nucleases specific for a given target can be readily selected by one of skill in the art. Nucleases often cleave both strands of dsDNA molecule within several bases of each other, resulting in a double-stranded break. Non-limiting examples of nucleases can include nucleases comprising a FokI cleavage domain, zinc finger nucleases, TALE nucleases, RNA-guided engineered nucleases, Cas9, Cas9-derived nucleases, homing endonucleases (e.g. I-AniI, I-CreI, and I-SceI) and the like. Further discussion of the various types of nucleases and how their site-specificity can be engineered can be found, e.g. in Silva et al. Curr Gene Ther 2011 11:11-27; Gaj et al. Trends in Biotechnology 2013 31:397-405; Humbert et al. Critical Reviews in Biochemistry and Molecular Biology 2012 47:264-281; and Kim and Kim Nature 2014 doi:10.1038/nrg3686; each of which is incorporated by reference herein in its entirety.

As used herein, “nickase” refers to a nuclease which cleaves only one strand of a dsDNA molecule, thereby generating a nick. Non-limiting examples of nickases can include a nuclease with one active site disabled; I-AniI with one active site disabled; or Cas9^{D10A}. Further discussion of nickases can be found, e.g. in Chan and Xu. NEB Expressions 2006 vol 1.2; Ramierez et al., Nucleic Acids Research 2012 40:5560-8; and Kim et al. Genome Research 2012 22:1327-1333; each of which is incorporated by reference herein in its entirety.

It is noted that nucleases and nickases can be readily engineered to target any given sequence. For example, Cas9-derived nucleases and nickases are targeted by means of guide nucleic acid molecules, which can be engineered to hybridize specifically to a desired target nucleic acid molecule. By way of further non-limiting example, zinc finger nucleases can be targeted by a combinatorial assembly of multiple zinc finger domains with known DNA triplet specificities. Such targeting approaches are known in the art and described, e.g. in Silva et al. Curr Gene Ther 2011 11:11-27; Ran et al. Cell 2013 154:1380-9; Jinek et al. Science 2013 337:816-821; Carlson et al. PNAS 212 109:17382-7, Guerts et al. Science 2009 325:433-3; Takasu et al. Insect Biochem Mol Biol 2010 40:759-765; and Watanabe et al. Nat. Commun. 2012 3; each of which is incorporated by reference herein in its entirety.

In some embodiments, the method can further comprise generating a nick in the nucleic acid molecule to be modified. In some embodiments, the method can further comprise generating a nick in the transcribed strand of the nucleic acid molecule to be modified. In some embodiments, the nick in the transcribed strand is generated by contacting the nucleic acid to be modified with a nickase specific for the transcribed strand of a dsDNA. As used herein, “transcribed strand” refers to the strand of a dsDNA which serves as the template for transcription. The transcribed strand may also be referred to herein by as the “template strand.” In a transcribable nucleic acid molecule of known sequence, one of skill in the art can readily distinguish a transcribed strand from its complement and/or by analyzing gene expression product sequences. A “transcribed strand” of a nucleic acid molecule to be modified and a donor nucleic acid molecule serving as a “template” for alternative HDR may share homology and/or complementarity but are not necessarily related and should not be conflated.

In one aspect, provided herein is a method of gene modification, the method comprising contacting the cell with a) a ssDNA donor nucleic acid molecule comprising the gene modification to be made in the cell; b) a nuclease; and c) an inhibitor of a gene expression product of a gene of Table 1. In some embodiments, the inhibitor can be an inhibitor of RAD51; BRCA2; PALB2 or SHFM1. In embodiments where the donor nucleic acid molecule is a single-stranded nucleic acid, the donor nucleic acid molecule comprises a portion complementary to one strand of the target nucleic acid molecule. In some embodiments, the nuclease can be a nickase. In some embodiments, the donor nucleic acid molecule can comprise a portion complementary to the strand of the target nucleic acid molecule that is not nicked by the nickase. In some embodiments, a portion of the donor nucleic acid molecule can specifically hybridize to the strand of the target nucleic acid molecule that is not nicked by the nickase, e.g. under the conditions under which the modification process will occur.

In some embodiments, the modification can be introduced as a gene therapy, e.g., to repair a mutation or defect in the DNA of a cell and/or subject. Such repairs can restore wild type and/or normal function of a gene and/or reduce harmful effects of a gene.

In some embodiments, the methods of gene modification can be performed in vivo. Alternatively, in some embodiments, the methods of gene modification can further comprise the step of implanting the modified cell in a subject. In some embodiments, the cell can be autologous to the subject. In some embodiments, the cell can be a stem cell, e.g. a somatic stem cell, a fetal stem cell, and/or an iPSC.

In some embodiments of the aspects described herein, the modification can correct a mutation. In some embodiments, a harmful or deleterious mutation is corrected, e.g. to the wildtype sequence and/or to a benign sequence. In some embodiments, modification can introduce a mutation. In some embodiments, a mutation can provide improved function. In some embodiments, a modification introduced according to the methods described herein can cause improved cell function. As used herein, “improved cell function” refers to an increase in at least one desirable activity that increases the productivity and/or survival of the cell or contributes positively to the health of an organism comprising the cell. In some embodiments, improved cell function can include a beneficial function the cell did not previously demonstrate or the loss of a deleterious function the cell did previously demonstrate. By way of non-limiting example, improved function can be accomplished by, e.g., modifying a viral gene or a gene comprising a dominant negative mutation. For example, a latent viral gene (e.g. HIV) can be modified (e.g. knocked-out or disabled). Another non-limiting example relates to collagen A mutations, which are often dominant negative. By specifically targeting a modification to the defective allele that prevented synthesis of proteins, collagen would become functional in the cell (e.g. a corrective modification and/or a modification which knocks out or knocks down the dominant negative allele).

In some embodiments of the foregoing aspects, the rate of mutatgenic end joining is not increased as a result of the method. In some embodiments of the foregoing aspects, the rate of mutatgenic end joining is not altered as a result of the method, e.g. it is neither increased nor decreased by a statistically significant amount. As used herein “mutagenic end-joining” refers to any DSB repair pathway that directly ligates the ends of DSB without the use of a homologous template, and results in at least one mutation arising relative to the original sequence. Mutagenic end joining can include, e.g., non-homologous end joining (NHEJ) and microhomology-mediated end joining (MMEJ).

Conversely, increasing the activity of the genes of Table 1 can decrease the rate of alternative HDR. Because of the mechanism of HDR, damaged DNA may be repaired using the second chromosome of a pair as the donor nucleic acid molecule. Such repair mechanisms can lead to a loss of heterozygosity, a leading cause of genomic instability in cancer. Accordingly, in order to prevent a loss of heterozygosity and decrease genomic instability, described herein is a method of decreasing genomic instability in a cell, the method comprising contacting the cell with an agonist of a gene of Table 1 or an inhibitor of BRCA1. In some embodiments, the agonist can be an agonist of RAD51; BRCA2; PALB2 or SHFM1. In some embodiments, the cell can be a cancerous cell.

As used herein, the term “BRCA1” refers to a gene encoding a polypeptide with a zinc finger domain and a BRCT domain, which is involved in DNA damage repair. BRCA1 binds to DNA and interacts directly with RAD51. Sequences for BRCA1 polypeptides and nucleic acids for a number of species are known in the art, e.g. human SHFM1 (NCBI Gene ID: 672) polypeptide (SEQ ID NO: 172; NCBI Ref Seq: NP_{—}009225) and nucleic acid (SEQ ID NO: 173; NCBI Ref Seq: NM_{—}007294).

As used herein, “agonist” refers to any agent that increases the level and/or activity of the target, e.g, of BRCA2. As used herein, the term “agonist” refers to an agent which increases the expression and/or activity of the target by at least 10% or more, e.g. by 10% or more, 50% or more, 100% or more, 200% or more, 500% or more, or 1000% or more. Non-limiting examples of agonists of BRCA2 can include BRCA2 polypeptides or fragments thereof and nucleic acids encoding a BRCA2 polypeptide, e.g. a polypeptide comprising the sequence SEQ ID NO: 095 or a nucleic acid comprising the sequence of SEQ ID NO: 009 or variants thereof. Fragments of BRCA2 which retain BRCA2 activity are known in the art, e.g. either the PALB2-interaction domain or the DNA-binding domain can be deleted and the resulting polypeptide retains activity. The structure of BRCA2, and fragments thereof that retain activity are described in more detail in, e.g. Siaud et al. PLoS Genetics 2011 7:e1002409; which is incorporated by reference herein in its entirety.

In some embodiments, an agonist can be a nucleic acid encoding the target, e.g. a nucleic aid encoding RAD51; BRCA2; PALB2 or SHFM1. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. DNA can include genomic DNA or cDNA. Other suitable nucleic acid molecules include RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a polypeptide as described herein (e.g. an BRCA2 polypeptide) can be comprised by a vector. In some of the aspects described herein, a nucleic acid sequence encoding a given polypeptide as described herein, or any module thereof, is operably linked to a vector. The term “vector”, as used herein, refers to a recombinant nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a gene expression product as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, “genomic instability” refers to the loss and/or alteration of genetic material. In some embodiments, genomic instability can be a loss of heterozygosity.

In some embodiments, the contacting step can comprise administering the agonist or inhibitor to a subject in need of treatment for a risk of genomic instability. In some embodiments, the subject can be a subject having or diagnosed as having cancer.

In some embodiments, the methods described herein relate to treating a subject having or diagnosed as having cancer with a composition as described herein, e.g. an agonist of a gene selected from Table 1 and/or an inhibitor of BRCA1. Subjects having cancer can be identified by a physician using current methods of diagnosing cancer. As used herein, the term “cancer” or “tumor” refers to an uncontrolled growth of cells which interferes with the normal functioning of the bodily organs and systems. A subject who has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs.

The compositions and methods described herein can be administered to a subject having or diagnosed as having cancer. In some embodiments, the methods described herein comprise administering an effective amount of compositions described herein to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a therapy needed to alleviate at least one or more symptom of the disease or disorder, and relates to a sufficient amount of pharmacological composition to provide the desired effect. The term “therapeutically effective amount” therefore refers to an amount of a therapy that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a CTC marker-gene targeted therapy, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., tumor growth, among others. The dosage can be determined by a physician and adjusted, as necessary, to suit observed effects of the treatment.

In some embodiments, the technology described herein relates to a pharmaceutical composition comprising a composition (e.g. an agonist of a gene selected from Table 1 or an inhibitor of BRCA1) as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition as described herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions comprising a composition described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the therapy can be administered in a sustained release formulation. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include radiation therapy, surgery, and chemotherapeutic agents.

In certain embodiments, an effective dose of a composition as described herein can be administered to a patient once. In certain embodiments, an effective dose of a composition comprising a composition described herein can be administered to a patient repeatedly. For systemic administration, subjects can be administered a therapeutic amount of a composition, such as, e.g. 10 μg/kg, 10 μg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. tumor growth by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the therapy. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The efficacy of a therapy in, e.g. the treatment of a condition described herein, or to induce a response as described herein (e.g. reduction of tumor growth) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of cancer, e.g. pancreatic cancer. When using an experimental animal model, efficacy of treatment is evidenced when a statistically significant change in a marker is observed, e.g. a change in the rate of tumor growth, genomic instability, LOH, or the rate of alternative HDR.

In one aspect, described herein is a kit comprising a nuclease or a nickase; and an inhibitor of a gene expression product of a gene of Table 1. In some embodiments, the inhibitor can be an inhibitor of RAD51; BRCA2; PALB2 or SHFM1. In some embodiments, the kit can further comprise a donor nucleic acid molecule. In some embodiments, nickase can be selected from the group consisting of: a nuclease with one active site disabled; I-AniI with one active site disabled; or Cas9^{D10A}. In some embodiments, the inhibitor can be an inhibitory nucleic acid. In some embodiments, the inhibitor can be an antibody reagent. In some embodiments, the inhibitor can be selected from the group consisting of: IBR2; RI-1; RI-2; and B02.

A kit is any manufacture (e.g., a package or container) comprising a nickase or nuclease as described herein and an inhibitor of a gene expression product of a gene of Table 1, according to the various embodiments herein, the manufacture being promoted, distributed, or sold as a unit for performing a methods as described herein. The kits described herein can optionally comprise additional components useful for performing the methods and assays described herein. Such reagents can include, e.g. a donor nucleic acid, transfection or vial packaging reagents, cell culture media, buffer solutions, labels, and the like. Such ingredients are known to the person skilled in the art and may vary depending on the particular cells and methods or assay to be carried out. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g. cancer or a condition in need of gene therapy. A subject can be male or female.

As used herein, the term “complementary” refers to the hierarchy of hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. As used herein, “substantially complementary” refers to a nucleic acid molecule or portion thereof having at least 90% complementarity over the entire length of the molecule or portion thereof with a second nucleotide sequence, e.g. 90% complementary, 95% complementary, 98% complementary, 99% complementary, or 100% complementary. As used herein, “substantially identical” refers to a nucleic acid molecule or portion thereof having at least 90% identity over the entire length of a the molecule or portion thereof with a second nucleotide sequence, e.g. 90% identity, 95% identity, 98% identity, 99% identity, or 100% identity.

As used herein, “specific” when used in the context of a sequence specific for a target nucleic acid refers to a level of complementarity between the donor nucleic acid molecule and the target such that there exists an annealing temperature at which the donor nucleic acid molecule will anneal to and mediate repair of the target nucleic acid and will not anneal to or mediate repair of non-target sequences present in a sample.

As used herein, a “portion” of a nucleic acid molecule refers to contiguous set of nucleotides comprised by that molecule. A portion can comprise any subset less than all nucleotides comprised by the reference nucleic acid molecule. A portion can be double-stranded or single-stranded.

The term “agent” refers generally to any entity which is normally not present or not present at the levels being administered to a cell, tissue or subject and which mediates or causes a desired effect within the context of a method as described herein. An agent can be selected from a group including but not limited to: polynucleotides; polypeptides; small molecules; and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein within the cell. In some embodiments, the agent is any chemical, entity or moiety, including without limitation synthetic and naturally-occurring non-proteinaceous entities that mediate or cause a desired effect within the context of a method as described herein. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected, on the basis of activity, from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

A subject can be one who has been previously diagnosed with or identified as suffering from or having a condition in need of treatment (e.g. cancer or a condition in need of gene therapy) or one or more complications related to such a condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for cancer or a condition in need of gene therapy or one or more complications related to cancer or a condition in need of gene therapy or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, a given “polypeptide”, e.g. a BRCA2 polypeptide, can include the human polypeptide as well as homologs from other species, including but not limited to bovine, dog, cat chicken, murine, rat, porcine, ovine, turkey, horse, fish, baboon and other primates. The terms also refer to fragments or variants of the wild-type polypeptide that maintain at least 50% of the activity or effect, of the full length wild-type polypeptide. Conservative substitution variants that maintain the activity of wildtype polypeptides will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of allograft rejection as described herein.

In some embodiments, a polypeptide, e.g., a BRCA2 polypeptide, can be a variant of a sequence described herein. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype polypeptide, e.g. 110%, 125%, 150%, 175%, 200%, 500%, 1000% or more.

One method of identifying amino acid residues which can be substituted is to align, for example, human polypeptide to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking Conversely, cysteine bond(s) can be added to the polypeptide to improve its stability or facilitate oligomerization.

In some embodiments, a polypeptide can comprise one or more amino acid substitutions or modifications. In some embodiments, the substitutions and/or modifications can prevent or reduce proteolytic degradation and/or prolong half-life of the polypeptide in the subject or cell. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide as described herein can comprise at least one peptide bond replacement. A polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide as described herein can comprise naturally occurring amino acids commonly found in polypeptides and/or proteins produced by living organisms, e.g. Ala (A), Val (V), Leu (L), Ile (I), Pro (P), Phe (F), Tip (W), Met (M), Gly (G), Ser (S), Thr (T), Cys (C), Tyr (Y), Asn (N), Gln (Q), Asp (D), Glu (E), Lys (K), Arg (R), and His (H). In some embodiments, a polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide can be modified, e.g. by addition of a moiety to one or more of the amino acids that together comprise the peptide. In some embodiments, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobin).

In some embodiments, a polypeptide can be a functional fragment of one of the amino acid sequences described herein. As used herein, a “functional fragment” is a fragment or segment of a polypeptide which retains at least 50%, at least 60%, at least 70%, at least 80%, at least 90% or more of the activity of the wildtype polypeptide, e.g., in any of the assays described herein. A functional fragment can comprise conservative substitutions of the sequences disclosed herein.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer or a condition in need of gene therapy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to an active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2011 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of increasing alternative homology-directed repair (HDR) at a target nucleic acid nick in a cell, the method comprising contacting the cell with an inhibitor of RAD51; BRCA2; PALB2 or SHFM1.
2. A method of modifying the sequence of a target nucleic acid molecule, the method comprising contacting the target nucleic acid molecule with
   a) a donor nucleic acid molecule comprising the modification to be made in the target nucleic acid molecule;
   b) a nickase; and
   c) an inhibitor of RAD51; BRCA2; PALB2 or SHFM1.
3. The method of paragraph 2, wherein a cell-free system comprises the target nucleic acid molecule.
4. The method of paragraph 2, wherein a cell comprises the target nucleic acid molecule.
5. The method of any of paragraphs 1-4, wherein the rate of mutagenic end joining is not increased.
6. The method of any of paragraphs 1-4, wherein the rate of mutagenic end joining is not altered.
7. The method of any of paragraphs 1-6, wherein the method further comprises generating a nick in the transcribed strand of the target nucleic acid molecule.
8. The method of any of paragraphs 2-7, wherein the nickase is selected from the group consisting of:
   a nuclease with one active site disabled; I-AniI with one active site disabled; or Cas9^{D10A}.
9. The method of any of paragraphs 2-8, wherein the donor nucleic acid molecule is a ssDNA or nicked dsDNA.
10. The method of any of paragraphs 2-9, wherein the donor nucleic acid molecule comprises a portion complementary to the strand of the target nucleic acid molecule that is not nicked by the nickase.
11. The method of paragraph 10, wherein the portion of the donor nucleic acid molecule that is complementary to a strand of the target nucleic acid molecule is substantially centered with respect to the location of the nick.
12. A method of modifying the sequence of a target nucleic acid molecule, the method comprising contacting the target nucleic acid molecule with
   a) a ssDNA donor nucleic acid molecule comprising the modification to be made in the target nucleic acid molecule;
   b) a nuclease; and
   c) an inhibitor of RAD51; BRCA2; PALB2 or SHFM1.
13. The method of paragraph 12, wherein a cell-free system comprises the target nucleic acid molecule.
14. The method of paragraph 12, wherein a cell comprises the target nucleic acid molecule.
15. The method of any of paragraphs 12-14, wherein the rate of mutagenic end joining is not increased.
16. The method of any of paragraphs 12-14, wherein the rate of mutagenic end joining is not altered.
17. The method of any of paragraphs 12-16, wherein the donor nucleic acid molecule comprises a portion complementary to one strand of the target nucleic acid molecule.
18. The method of any of paragraphs 12-17, wherein the nuclease is selected from the group consisting of:
   nucleases comprising a FokI cleavage domain; zinc finger nucleases; TALE, nucleases; RNA-guided engineered nucleases; Cas9; Cas9-derived nucleases; and horning endonucleases.
19. The method of any of paragraphs 1-18, wherein the modification is introduced as a gene therapy.
20. The method of any of paragraphs 1-19, wherein the inhibitor is an inhibitory nucleic acid.
21. The method of any of paragraphs 1-19, wherein the inhibitor is an antibody reagent.
22. The method of any of paragraphs 1-19, wherein the inhibitor is selected from the group consisting of:
   IBR2; RI-1; RI-2; and B02.
23. The method of any of paragraphs 2-22, wherein the donor nucleic acid molecule is at least about 25 nt in length.
24. The method of any of paragraphs 2-23, wherein the donor nucleic acid molecule is at least about 50 nt in length.
25. The method of any of paragraphs 1-24, further comprising the step of implanting a cell comprising the modified nucleic acid molecule into a subject.
26. The method of paragraph 25, wherein the cell is autologous to the subject.
27. The method of any of paragraphs 1-26, wherein the cell is an iPS cell.
28. The method of any of paragraphs 1-27, wherein the modification corrects a mutation.
29. The method of any of paragraphs 1-27, wherein the modification introduces a mutation.
30. The method of any of paragraphs 1-29, wherein the modification causes improved cell function.
31. The method of paragraph 30, wherein the modification is selected from the group consisting of:
   modification of a viral gene and modification of a gene comprising a dominant negative mutation.
32. A method of decreasing genomic instability in a cell, the method comprising contacting the cell with an agonist of RAD51; BRCA2; PALB2 or SHFM1 or an inhibitor of BRCA1.
33. The method of paragraph 32, wherein the agonist is a nucleic acid encoding RAD51; BRCA2; PALB2 or SHFM1.
34. The method of paragraph 32, wherein the inhibitor is an inhibitory nucleic acid.
35. The method of paragraph 32, wherein the inhibitor is an antibody reagent.
36. The method of any of paragraphs 32-35, wherein the cell is a cancerous cell.
37. The method of any of paragraphs 32-36, wherein the genomic instability is a loss of heterozygosity.
38. The method of any of paragraphs 32-37, wherein the contacting step comprises administering the agonist or inhibitor to a subject in need of treatment for a risk of genomic instability.
39. The method of paragraph 38, wherein the subject is a subject with cancer.
40. A kit comprising:
   a nuclease or a nickase; and
   an inhibitor of RAD51; BRCA2; PALB2 or SHFM1.
41. The kit of paragraph 40, further comprising a donor nucleic acid molecule.
42. The kit of paragraph 41, wherein the donor nucleic acid molecule is a single-stranded nucleic acid molecule.
43. The kit of any of paragraphs 40-42, wherein the nickase is selected from the group consisting of:
   a nuclease with one active site disabled; I-AniI with one active site disabled; or Cas9^{D10A}.
44. The kit of any of paragraphs 40-42, wherein the nuclease is selected from the group consisting of:
   nucleases comprising a FokI cleavage domain; zinc finger nucleases; TALE nucleases; RNA-guided engineered nucleases; Cas9; Cas9-derived nucleases; and homing endonucleases.
45. The kit of any of paragraphs 40-44, wherein the inhibitor is an inhibitory nucleic acid.
46. The kit of any of paragraphs 40-45, wherein the inhibitor is an antibody reagent.
47. The kit of any of paragraphs 40-46, wherein the inhibitor is selected from the group consisting of:
   IBR2; RI-1; RI-2; and B02.
48. The kit of any of paragraphs 40-47, further comprising a cell extract.

### EXAMPLES

### Example 1

### Homology-Directed Repair of DNA Nicks

Nicks are the most common form of DNA damage and, if unrepaired, can give rise to genomic instability. Nicks can be caused by oxidative stress or irradiation and are transient intermediates in base excision repair, nucleotide excision repair, and mismatch repair. Nicks can be repaired via the single-strand break repair pathway¹ but may also initiate homology-directed repair (HDR)²⁻⁶. It is demonstrated herein that, in human cells, HDR at DNA nicks occurs via a novel pathway that is distinct from canonical double-strand break (DSB) repair. HDR at nicks is characterized by two kinds of strand asymmetry not observed at DSBs: HDR was most efficient at a nick in the transcribed strand of a transcribed gene, and preferentially used a single-stranded DNA (ssDNA) donor complementary to the intact strand. HDR at nicks using either a ssDNA or nicked duplex DNA donor was stimulated upon downregulation of RAD51 or BRCA2. Efficiency of HDR at nicks can reach levels comparable to canonical HDR at DSBs, but associated local mutagenesis is much lower, so nick-initiated HDR can be applied to gene correction and genome engineering. The alternative HDR pathway that promotes repair at nicks can be activated in BRCA2-deficient tumors or in other contexts in which canonical HDR is compromised or impaired.

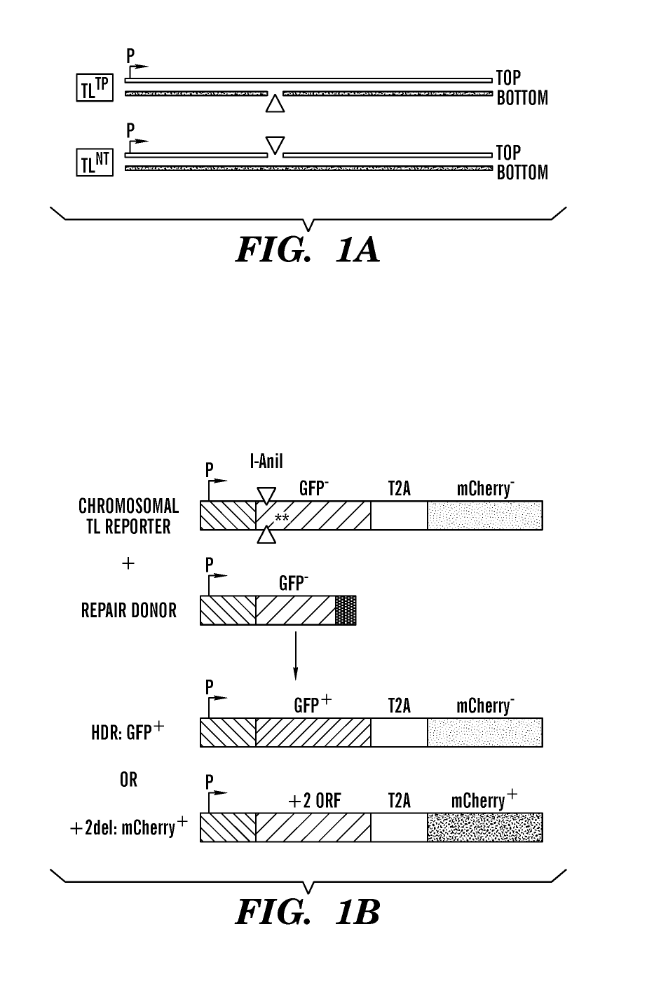

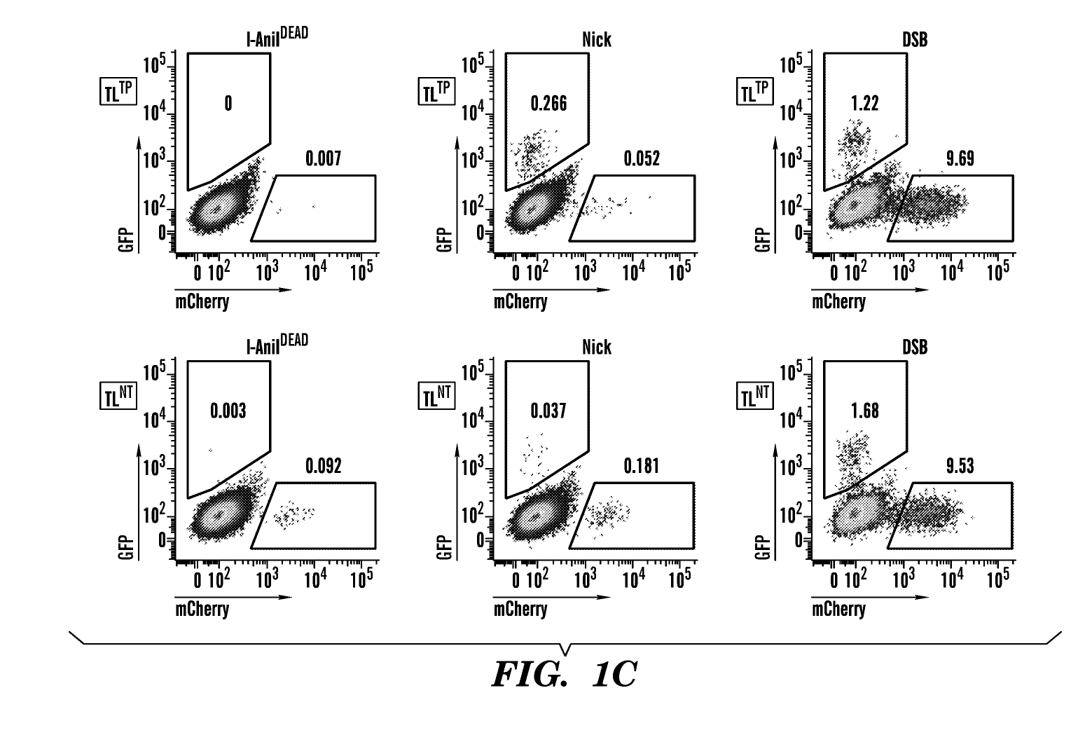

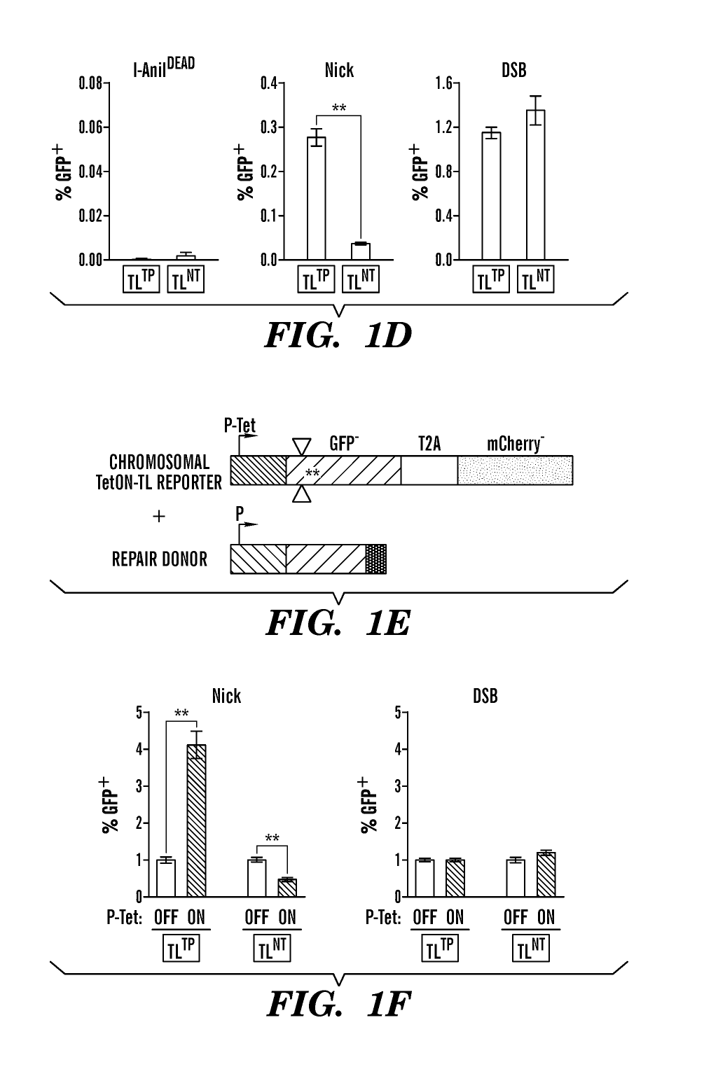

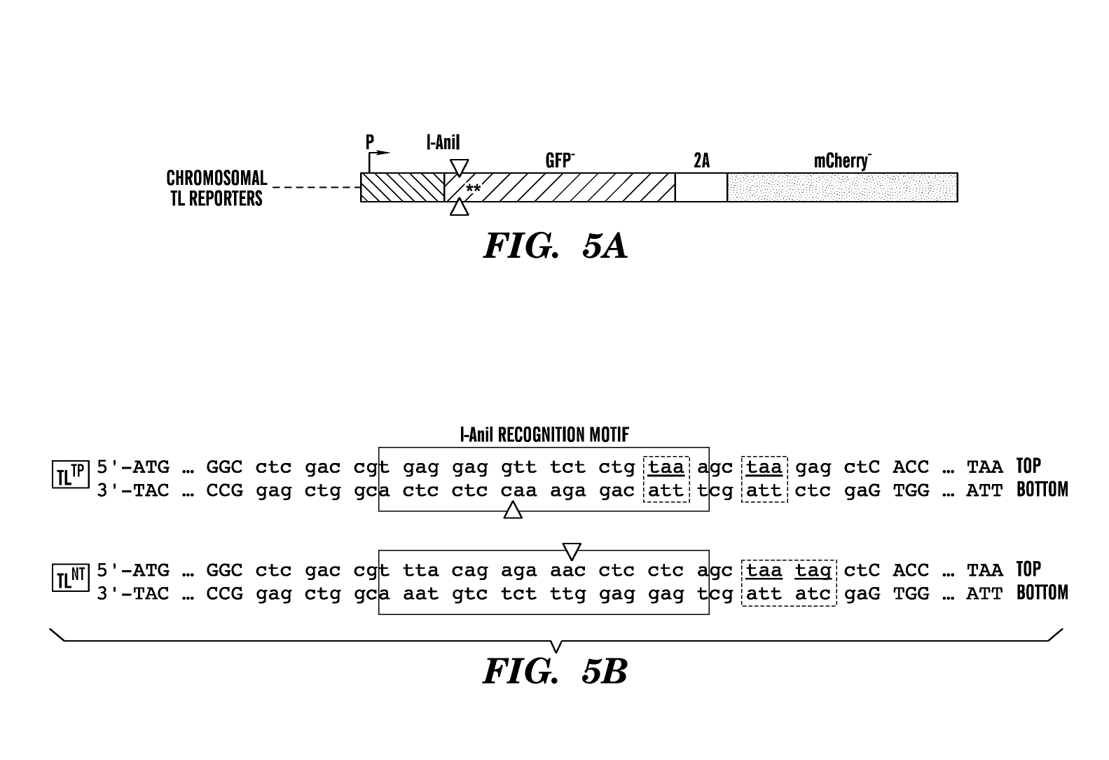

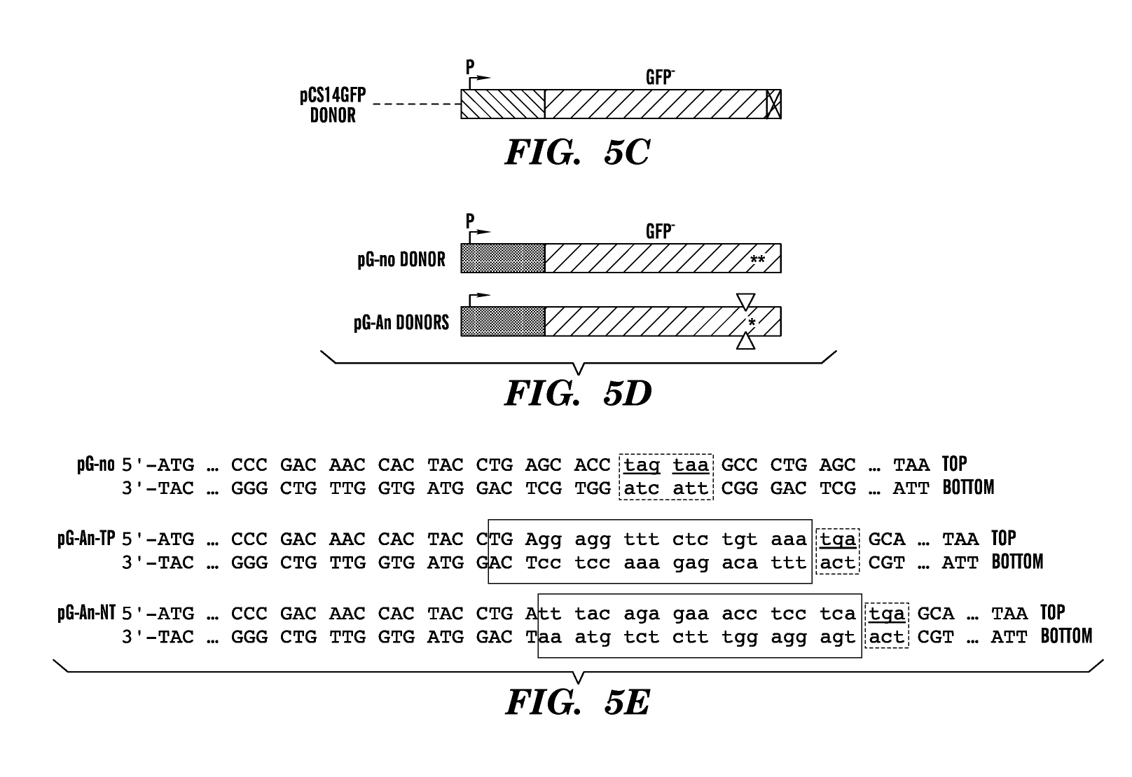

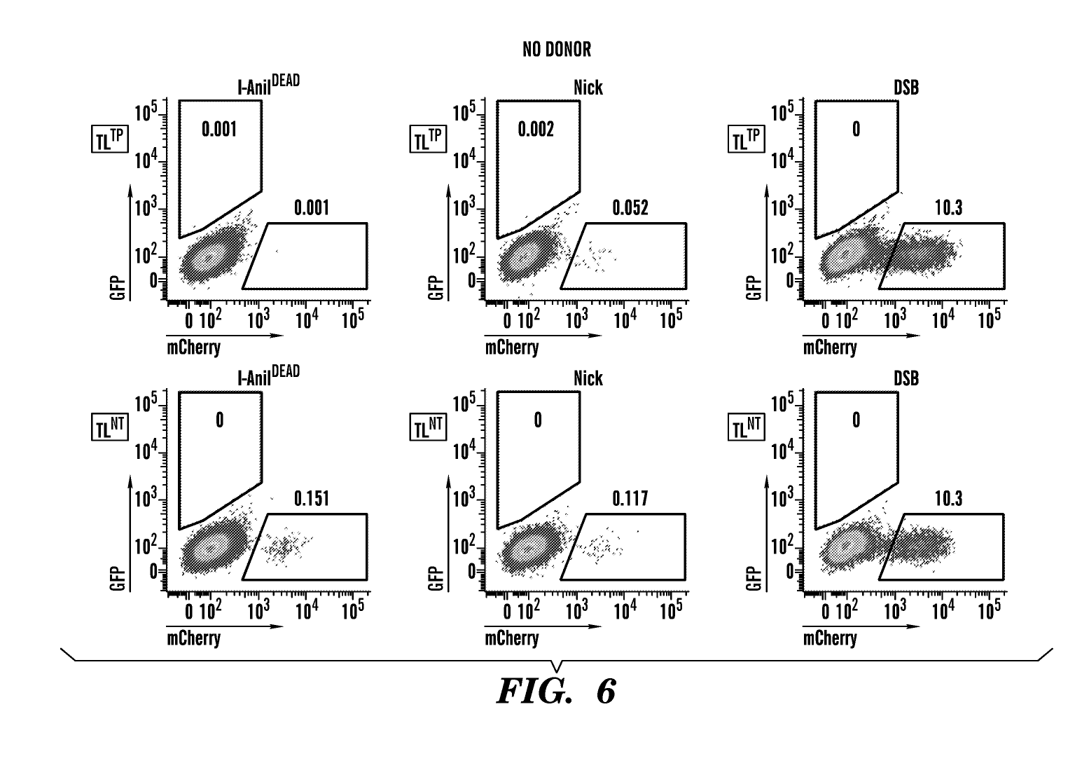

Damage to the transcribed DNA strand is preferentially detected and repaired in transcription-coupled nucleotide excision repair⁷, and transcribedstrand nicks can arrest transcriptional elongation in human cell extracts⁸. To ask if nick-initiated HDR at a transcribed gene exhibited similar strand bias, a “nickase” derivative of the I-AniI homing endonuclease, disabled at one of its two active sites so it cleaves a single DNA strand to generate a nick rather than a DSB⁵ was used. Chromosomal repair targets were derived from the TL reporter⁹. TLTP and TLTP reporters carry an I-AniI site oriented for nicking the transcribed or non-transcribed strand, respectively (FIGS. 1A and 5A-5E). HDR that replaces the I-AniI site and nonsense codons yields GFP+ cells (FIGS. 1B and 5A-5E). Populations of 293T cells bearing either the TLTP or TLNT reporter at heterogeneous integration sites were transiently transfected with dsDNA plasmid donor pCS14GFP (FIGS. 5A-5E) and a construct coexpressing I-AniI and BFP. GFP+ cells among I-AniI-expressing (BFP+) cells were quantified 3 days later. No GFP+ cells were generated following either expression of I-AniI in the absence of donor DNA (FIG. 6) or following expression of catalytically inactive I-AniI in the presence of donor (FIGS. 1C-1D). Nicks initiated HDR with a nearly 8-fold greater frequency in the TLTP than in the TLNT population, while DSBs initiated HDR at comparable frequencies in both (FIGS. 1C-1D).

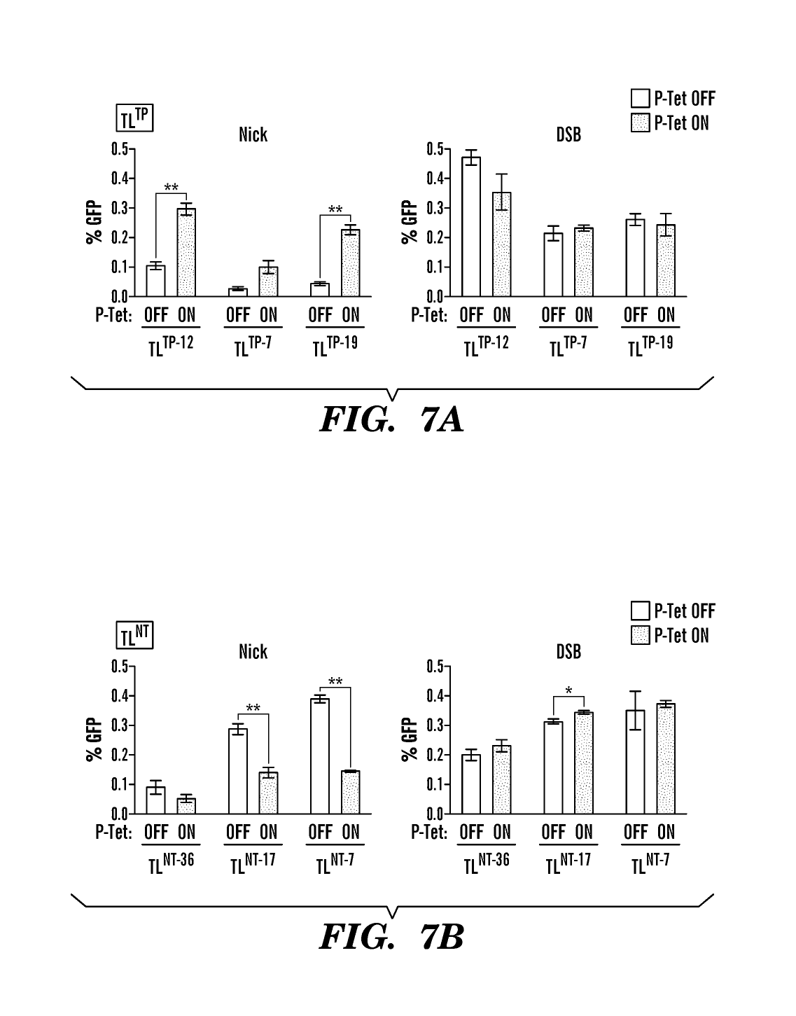

It is established herein that transcription causes the strand bias in HDR at nicks using reporters in which the GFP gene is tetracycline-inducible (P-Tet; FIG. 1E). Induction of transcription increased HDR 4-fold at a transcribed strand nick, and reduced HDR 2-fold at a non-transcribed strand nick, but had no effect at a DSB (FIGS. 1F and 7). This strand bias may reflect functions of the transcription apparatus in detecting a nick, preventing its immediate religation, or promoting HDR.

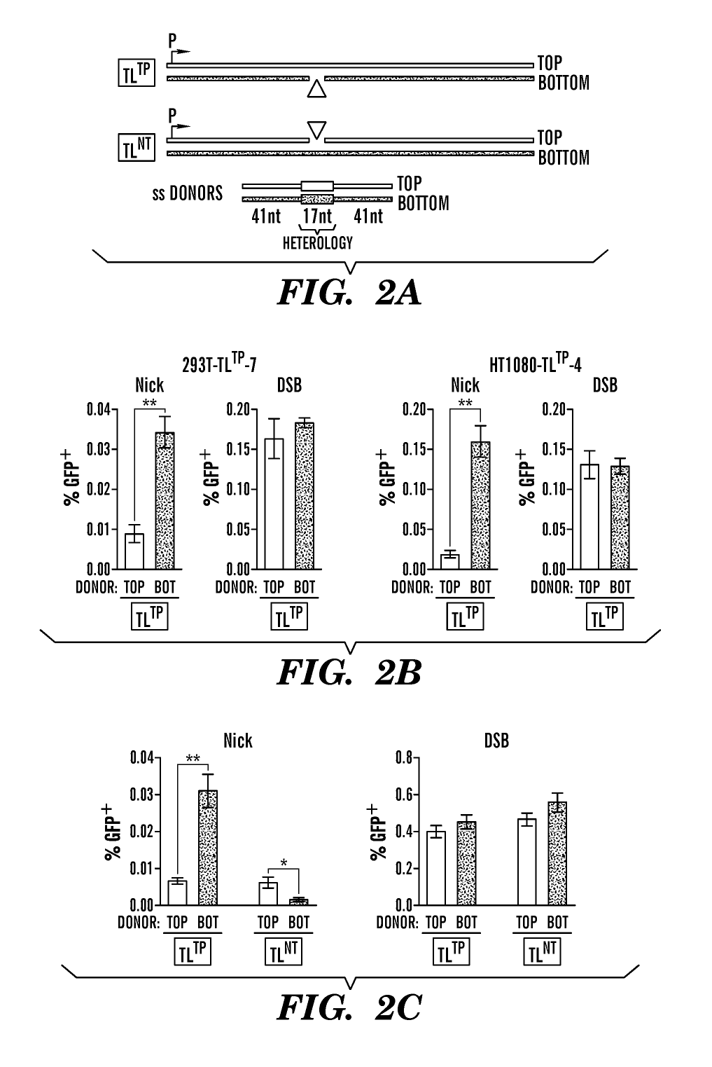

ssDNA can support HDR at DSBs¹⁰. It was determined if the inherent asymmetry of a nick biases donor strand utilization, using 99 nt ssDNA donors in which a central 17 nt heterologous region supports repair of the defective target gene (FIG. 2A). In clonal derivatives of either 293T or HT1080 cells carrying the TLTP reporter, nicks were more efficiently repaired by a ssDNA donor complementary to the intact chromosomal strand, while no donor strand bias was evident at DSBs (FIG. 2B). A ssDNA donor complementary to the intact strand supported HDR more efficiently regardless of whether the initiating nick was on the transcribed or non-transcribed strand, while no donor strand bias was evident in HDR at DSBs (FIG. 2C). Thus the direction of transcription determines the efficiency of HDR but does not affect donor strand bias, which can instead depend upon interactions between the donor DNA and its target. Donor strand bias evident with exogenously-supplied synthetic oligonucleotides can extend to intracellular repair of nicks by ssDNA donors derived from strands released by helicases, strand-displacement by DNA polymerases, or nucleolytic processing of structures formed during replication or recombination.

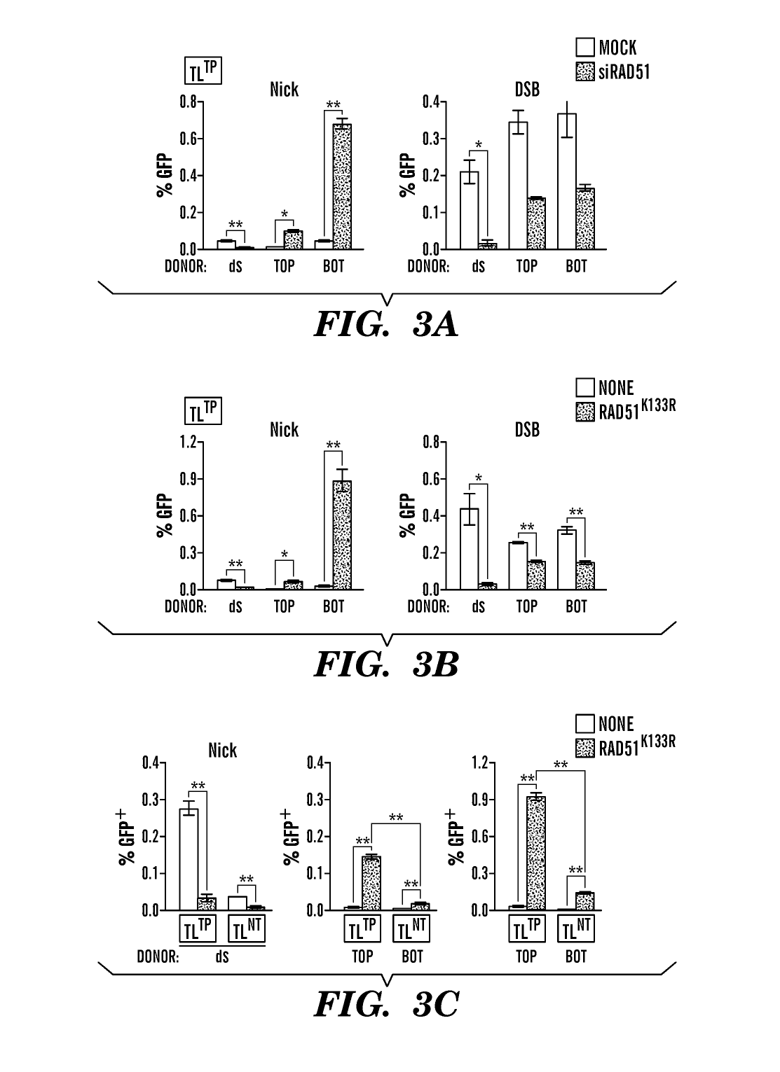

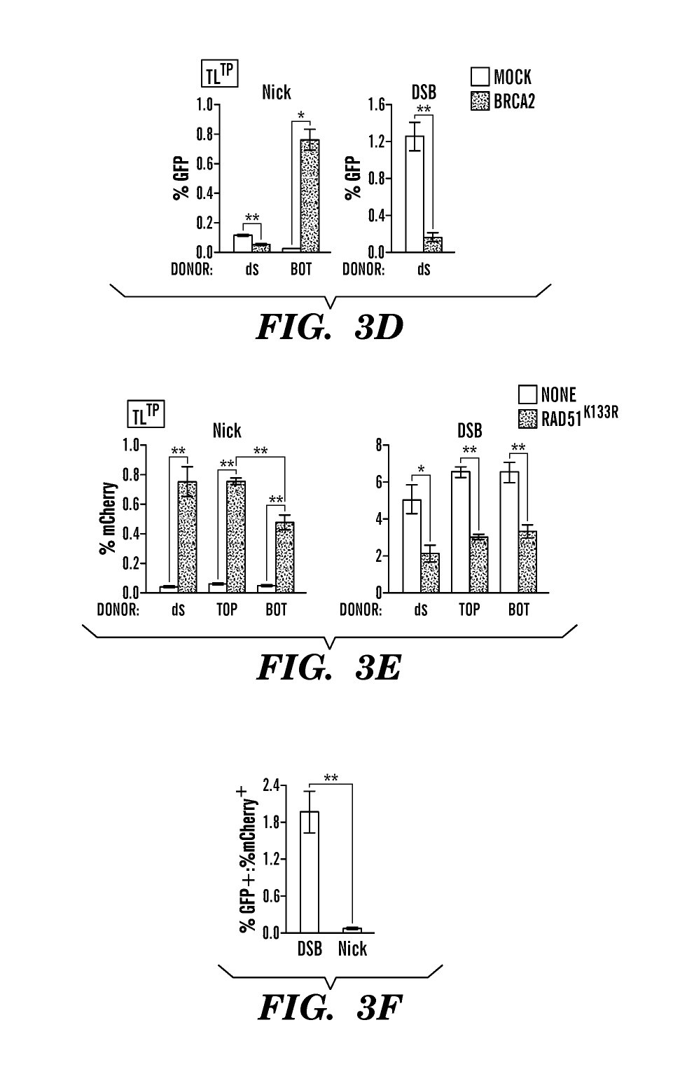

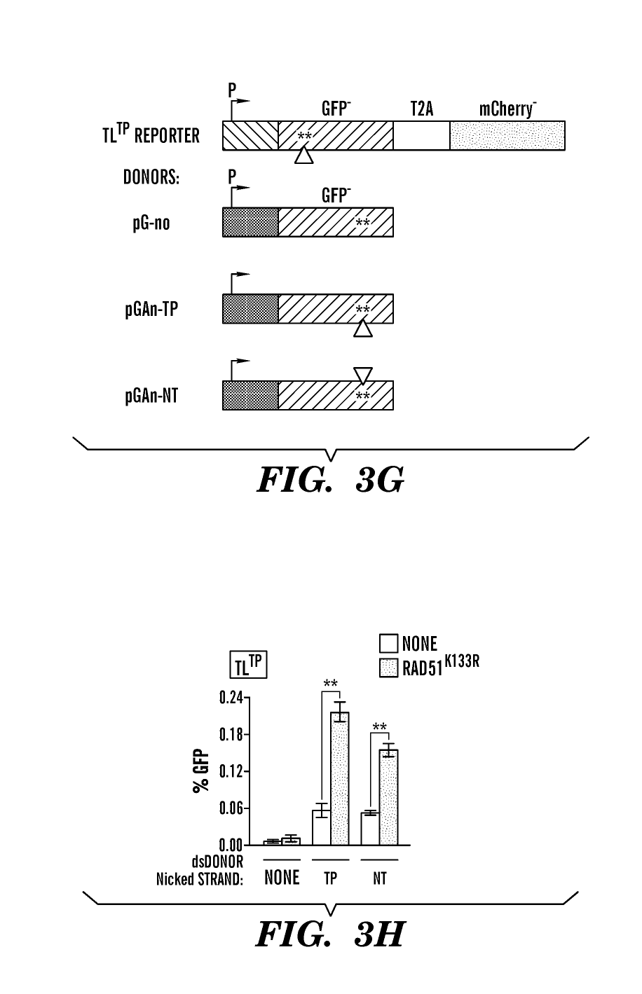

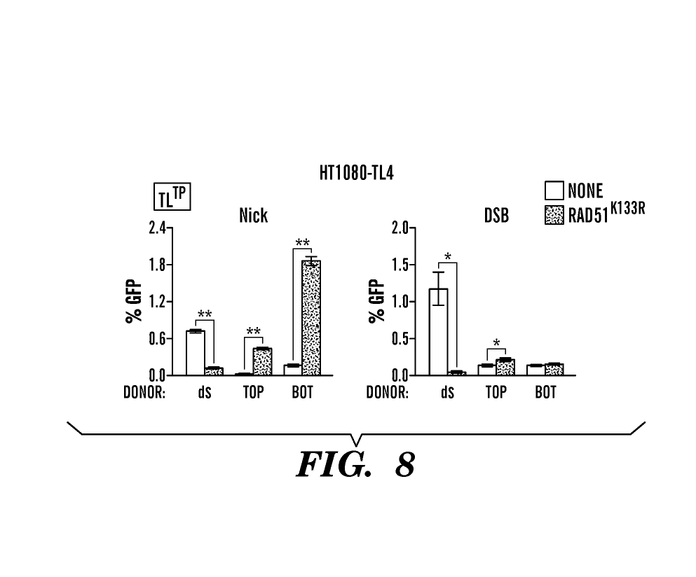

In the canonical HDR pathway, BRCA2 assembles factors including RAD51, which promotes strand exchange¹¹. Strikingly, siRAD51 treatment of the clonal 293T-TL7TP line greatly increased the frequency of HDR at nicks by either ssDNA donor, but reduced the frequency of HDR at nicks by the dsDNA donor (FIG. 3A). siRAD51 treatment reduced the frequency of HDR at DSBs, as expected, but with much greater effect on HDR by dsDNA than ssDNA donors (FIG. 3A). Transient expression of the RAD51K133R dominant negative mutant¹² had a similar effect as siRAD51 treatment, stimulating HDR by either ssDNA donor at nicks but not DSBs, in both the 293T-TL7TP (FIG. 3B) and the HT1080-TL4TP (FIG. 8) clonal lines. Stimulation was evident at nicks in either the transcribed or non-transcribed strand (FIG. 3C) Inhibition of BRCA2, by treatment of 293T-TL7TP cells with siBRCA2, similarly stimulated HDR at nicks using a ssDNA donor, but inhibited HDR at DSBs (FIG. 3D). Thus, HDR at nicks proceeds by an alternative pathway, which is normally inhibited by the canonical RAD51/BRCA2-dependent pathway and activated upon downregulation of factors in that pathway.

Activity of the alternative HDR pathway is not restricted to nicks. Upon RAD51-inhibition, ssDNA donors supported significant HDR at DSBs (50% in the 293T-TL7TP reporter line and 100% in the HT1080-TL4TP line; FIGS. 3A, 3B, and 8). Thus the alternative pathway can also support HDR at DSBs by ssDNA donors.

Strikingly, HDR frequencies at nicks in cells in which RAD51 or BRCA2 was inhibited (FIGS. 3A-3D) were comparable to HDR frequencies at DSBs, indicating that this alternative HDR pathway can provide a useful strategy for some genome engineering applications. Damage associated with gene targeting must be minimized for these applications. While the sequence-specificity of I-AniI limits its application to genome engineering, it is useful as a model in this context, as it cuts DNA to generate 5′-phosphate and 3′-hydroxyl ends like the FokI cleavage domain¹³ of zinc finger or TALE nucleases suited for genome engineering applications. The TL reporter was used to quantify mutagenic end joining (EJ) accompanied by +2 frameshift that allows the mCherry gene to be translated (FIG. 1B). In the 293T-TL7TP line, the frequency of nick-initiated mutagenic EJ was nearly two orders of magnitude lower than that of DSB-initiated mutagenic EJ, regardless of donor (FIG. 3E). RAD51K133R expression increased the frequency of mutagenic EJ, but the frequency of nick-initiated mutagenic EJ remained lower than that of DSB-initiated mutagenic EJ (FIG. 3E). At optimum conditions for HDR (nicks: transcribed-strand nick, bottom strand donor, RAD51 inhibited; DSBs: dsDNA donor), nick-initiated events exhibited a 20-fold higher ratio of HDR to mutagenic EJ than DSB-initiated events (% GFP:% mCherry cells; FIG. 3F). Thus, nick repair by the alternative HDR pathway occurs with less associated damage than DSB repair.

It was determined if alternative HDR could use a nicked dsDNA donor¹⁴ using plasmid donors carrying an I-AniI site at the 3′-end of the defective GFP gene, on either the transcribed or non-transcribed strand (FIG. 3G). Nicked dsDNA donors were 8-fold more active than the intact donor in 293T-TL7TP cells, regardless of whether the nick was on the transcribed or non-transcribed strand; and their activity was 3-fold further increased upon inhibition of RAD51 (FIG. 3H). Nicked dsDNA is therefore an active donor for the alternative HDR pathway. These results indicate that genomic dsDNA that has been nicked in the course of replication, transcription, recombination or repair may serve as an intracellular donor for nick repair by the alternative HDR pathway.

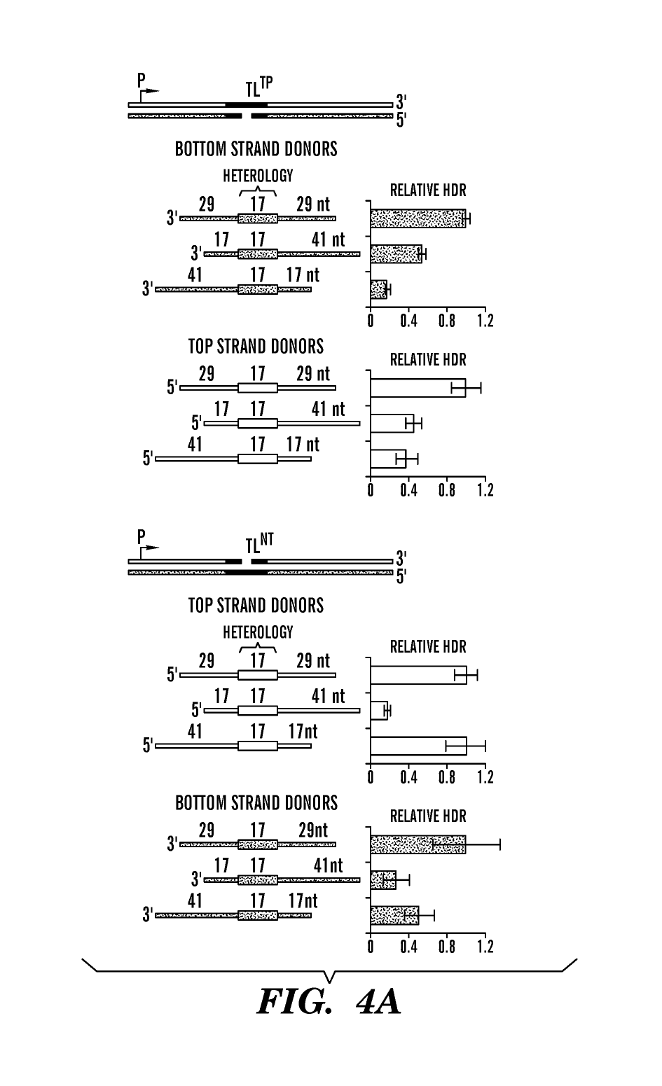

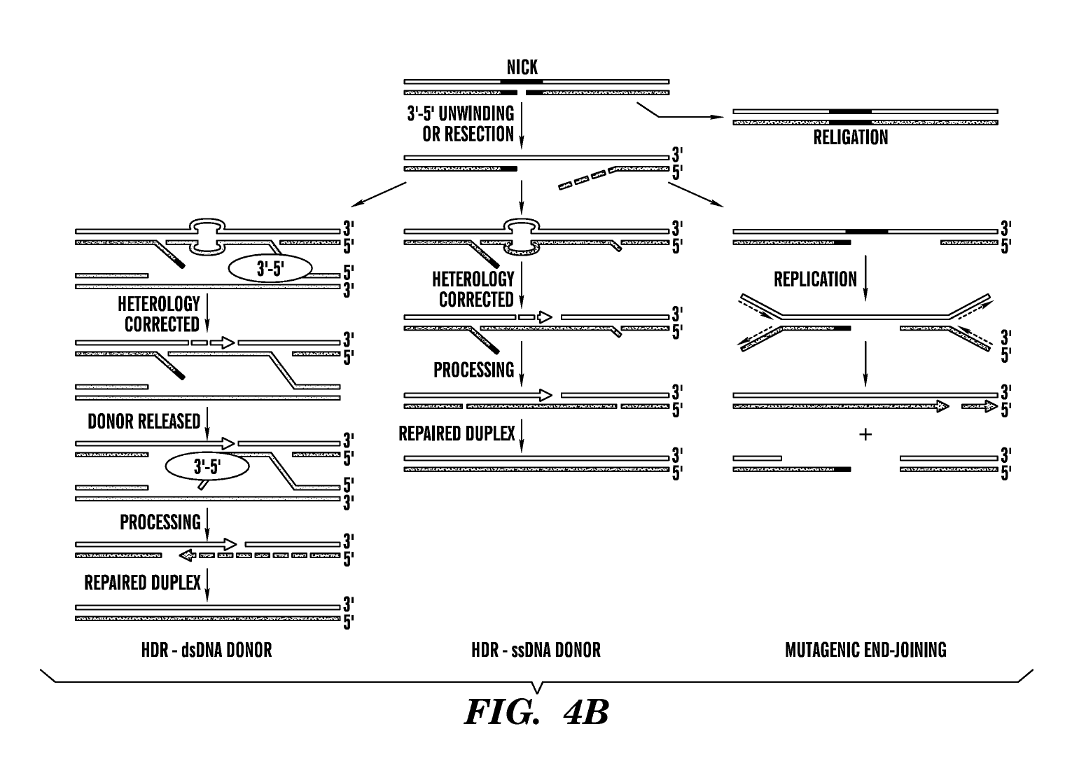

Preferential repair by a ssDNA donor complementary to the intact strand (FIGS. 2A-2C and 3A-3H) indicated an HDR mechanism in which the region near the nick is made accessible for donor annealing by unwinding or resection at the nick. It was determined if a predominant directionality characterized nick repair, by comparing HDR frequencies at nicks repaired by ssDNA donors identical in length (75 nt) and centered on the nick or extending upstream or downstream of the nick. Nick-initiated HDR at both transcribed and non-transcribed strand nicks was significantly less efficient if the region of extended homology was 3′ of the nick, independent of donor strandedness (FIG. 4A).

The results above lead to a working model for nick repair by the alternative HDR pathway. Without wishing to be bound by theory, the region 5′ of the nick may be exposed by the activity of a 3′-5′ helicase or exonuclease (FIG. 4B, top). DNA may then anneal to the exposed region independent of RAD51 (FIG. 4B, left); a similar step occurs in DSB repair at genomic repeats mediated by the single-strand annealing pathway¹⁵⁻¹⁸. Heterology is corrected and the donor released by a helicase or strand displacement. Processing generates an intact duplex with the corrected sequence at the target site. Release of the donor would preserve its integrity, so this model may apply to gene conversion at a DNA nick, as occurs at diversifying immunoglobulin genes¹⁹. An analogous pathway could carry out repair using a ssDNA donor strand (FIG. 4B, center). Alternatively, without wishing to be bound by theory, mutagenic end-joining, may occur when replication forks collide at the processed nick to generate two one-sided DSBs (FIG. 4Bt); fork stabilization by RAD51 and BRCA2 may inhibit these events²⁰. Nonetheless, the transcriptional asymmetry and donor strand bias of HDR at nicks shows that such DSBs are not an obligatory intermediate in repair by HDR.

Nicks occur frequently, underscoring the biological significance of their repair by HDR. Strikingly, the alternative HDR pathway is not just independent of but normally repressed by the canonical HDR pathway. Alternative HDR may thus be active in disease contexts in which canonical HDR is inactive, such as breast and ovarian cancers bearing BRCA2 mutations, or regions of solid tumors in which local hypoxic conditions downregulate canonical HDR21-24. The presence of nicks in both target and donor stimulated HDR via the alternative pathway, raising the intriguing possibility that HDR between nicked homologs may contribute to loss-of-heterozygosity events that drive malignancy.

Methods

Flow cytometry data were analyzed using FlowJo™ (Tree Star, Ashland, Oreg.) flow cytometry analysis software and frequencies were transferred to Microsoft Excel™ in which statistical significance was determined by two-tailed t-test. In all experiments I-AniI was co-expressed with mTagBFP (BFP) and GFP+ and mCherry+ frequencies among BFP+ cells are shown.

### REFERENCES

1. Caldecott, K. W. Single-strand break repair and genetic disease. Nat Rev Genet 9, 619-631 (2008).
2. Strathern, J. N., Weinstock, K. G., Higgins, D. R. & McGill, C. B. A novel recombinator in yeast based on gene II protein from bacteriophage f1. Genetics 127, 61-73 (1991).
3. Arcangioli, B. A site- and strand-specific DNA break confers asymmetric switching potential in fission yeast. EMBO J 17, 4503-4510 (1998).
4. Lee, G. S., Neiditch, M. B., Salus, S. S. & Roth, D. B. RAG proteins shepherd double-strand breaks to a specific pathway, suppressing error-prone repair, but RAG nicking initiates homologous recombination. Cell 117, 171-184 (2004).
5. McConnell Smith, A. et al. Generation of a nicking enzyme that stimulates site-specific gene conversion from the I-AniI LAGLIDADG (SEQ ID NO: 218) homing endonuclease. Proc Natl Acad Sci USA 106, 5099-5104 (2009).
6. Davis, L. & Maizels, N. DNA nicks promote efficient and safe targeted gene correction. PLoS One 6, e23981 (2011).
7. Hanawalt, P. C. & Spivak, G. Transcription-coupled DNA repair: two decades of progress and surprises. Nat Rev Mol Cell Biol 9, 958-970 (2008).
8. Kathe, S. D., Shen, G. P. & Wallace, S. S. Single-stranded breaks in DNA but not oxidative DNA base damages block transcriptional elongation by RNA polymerase II in HeLa cell nuclear extracts. J Biol Chem 279, 18511-18520 (2004).
9. Certo, M. T. et al. Tracking genome engineering outcome at individual DNA breakpoints. Nat Methods 8, 671-676 (2011).
10. Chen, F. et al. High-frequency genome editing using ssDNA oligonucleotides with zinc-finger nucleases. Nat Methods 8, 753-755 (2011).
11. Heyer, W. D., Ehmsen, K. T. & Liu, J. Regulation of homologous recombination in eukaryotes. Annu Rev Genet 44, 113-139 (2010).
12. Stark, J. M. et al. ATP hydrolysis by mammalian RAD51 has a key role during homology-directed DNA repair. J Biol Chem 277, 20185-20194 (2002).
13. Sanders, K. L., Catto, L. E., Bellamy, S. R. & Halford, S. E. Targeting individual subunits of the FokI restriction endonuclease to specific DNA strands. Nucleic Acids Res 37, 2105-2115 (2009).
14. Goncalves, M. A., van Nierop, G. P., Holkers, M. & de Vries, A. A. Concerted nicking of donor and chromosomal acceptor DNA promotes homology-directed gene targeting in human cells. Nucleic Acids Res 40, 3443-3455 (2012).
15. Bennardo, N., Cheng, A., Huang, N. & Stark, J. M. Alternative-NHEJ is a mechanistically distinct pathway of mammalian chromosome break repair. PLoS Genet 4, e1000110 (2008).
16. Ira, G. & Haber, J. E. Characterization of RAD51-independent break-induced replication that acts preferentially with short homologous sequences. Mol Cell Biol 22, 6384-6392 (2002).
17. Stark, J. M., Pierce, A. J., Oh, J., Pastink, A. & Jasin, M. Genetic steps of mammalian homologous repair with distinct mutagenic consequences. Mol Cell Biol 24, 9305-9316 (2004).
18. Weinstock, D. M., Richardson, C. A., Elliott, B. & Jasin, M. Modeling oncogenic translocations: distinct roles for double-strand break repair pathways in translocation formation in mammalian cells. DNA Repair (Amst) 5, 1065-1074 (2006).
19. Maizels, N. Immunoglobulin gene diversification. Annu Rev Genet 39, 23-46 (2005).
20. Schlacher, K., Wu, H. & Jasin, M. A distinct replication fork protection pathway connects Fanconi anemia tumor suppressors to RAD51-BRCA1/2. Cancer Cell 22, 106-116 (2012).
21. Bindra, R. S. et al. Down-regulation of Rad51 and decreased homologous recombination in hypoxic cancer cells. Mol Cell Biol 24, 8504-8518 (2004).
22. Meng, A. X. et al. Hypoxia down-regulates DNA double strand break repair gene expression in prostate cancer cells. Radiother Oncol 76, 168-176 (2005).
23. Bristow, R. G. & Hill, R. P. Hypoxia and metabolism. Hypoxia, DNA repair and genetic instability. Nat Rev Cancer 8, 180-192 (2008).
24. Klein, T. J. & Glazer, P. M. The tumor microenvironment and DNA repair. Semin Radiat Oncol 20, 282-287 (2010).
25. Smith, G. R. How homologous recombination is initiated: unexpected evidence for single-strand nicks from V(D)J site-specific recombination. Cell 117, 146-148 (2004). Supplementary Information is linked to the online version of the paper at www.nature.com/nature.

### Example 2

### Homology-Directed Repair of DNA Nicks Via Pathways Distinct from Canonical Double-Strand Break Repair

DNA nicks are the most common form of DNA damage, and if unrepaired can give rise to genomic instability. In human cells, nicks are efficiently repaired via the single-strand break repair (SSBR) pathway, but relatively little is known about the fate of nicks not processed by that pathway. Here we show that homology-directed repair (HDR) at nicks occurs via a mechanism distinct from HDR at double-strand breaks (DSBs). HDR at nicks, but not DSBs, is associated with transcription, and HDR is 8 fold more efficient at a transcribed than a non-transcribed strand nick. HDR at nicks can proceed by a pathway dependent upon RAD51 and BRCA2, similar to canonical HDR at DSBs; or by an efficient alternative pathway that uses either ssDNA or nicked dsDNA donors and that is strongly inhibited by RAD51 and BRCA2. Nicks generated by either I-AniI or the CRISPR/Cas9^{D10A} nickase are repaired by the alternative HDR pathway with little accompanying mutagenic end-joining, so this pathway may be usefully applied to genome engineering. These results indicate that alternative HDR at nicks may be stimulated in physiological contexts in which canonical RAD51/BRCA2-dependent HDR is compromised or downregulated, which occurs frequently in tumors. HDR at nicks can contribute to loss of heterozygosity, a common form of genomic instability in cancer.

Nicks are a very common form of DNA damage, but their threat to genomic integrity has been neglected as it is assumed that all nicks are repaired by simple religation. That assumption is challenged herein. Identified herein is a robust pathway for homology-directed repair that is active at DNA nicks. This alternative HDR pathway is stimulated upon downregulation of BRCA2 or RAD51, key factors in canonical HDR at double-strand breaks. Alternative HDR at targeted nicks has immediate practical applications to genome engineering. The alternative HDR pathway promotes repair of a nicked target by a nicked donor, and may thereby contribute to loss-of-heterozygosity, a common form of genomic instability in tumors.

DNA nicks (single-strand breaks) are the most common form of DNA damage. Every day tens of thousands of DNA nicks occur and are repaired in each cell (1). Nicks can be caused by oxidative stress or ionizing radiation, which generates 30 nicks for every double-strand break (DSB). Reactive oxygen species (ROS), such as superoxide, hydrogen peroxide, and hydroxyl radicals can damage a deoxyribose moiety to nick DNA directly, or modify DNA precursors (e.g. by converting guanine to 8-oxo-guanine) and thereby overload downstream repair to create a burden of nicked DNA (1-4). Nicks are also intermediates in essential DNA metabolism and repair pathways, including base excision repair (BER), nucleotide excision repair (NER), mismatch repair (MMR), rNMP removal, and regulation of superhelicity by topoisomerases.

Nicks are efficiently repaired by the single-strand break repair (SSBR) pathway, which assembles a repair complex at a nick in which XRCC1 is a critical but non-catalytic member (5-8). XRCC1 interacts with factors that clean up modified DNA ends to create a gap that is filled by POL β, or the replicative polymerases POL δ and ε. LIG3 or other ligases then reseal the DNA backbone (5-7).

Nicks can also initiate homology-directed repair (HDR) (9-12). This has drawn considerable interest as a strategy for gene therapy by targeted gene correction, because nicks cause less mutagenic end joining (mutEJ) than do DSBs (13, 14). However, the mechanism of HDR at nicks has not been defined, either in mammalian cells or in model organisms such as S. cerevisiae. In particular, it is not known if HDR at nicks proceeds via the canonical HDR pathway that has been characterized in detail at DNA double-strand breaks (DSBs), in which free single-stranded 3′ ends are exposed, allowing BRCA2 to load RAD51 thus promoting strand invasion (15).

It is demonstrated herein that, in human cells, HDR at DNA nicks, but not DSBs, is associated with transcription and occurs more efficiently at a transcribed strand nick than at a non-transcribed strand nick. HDR at nicks can occur via two pathways. One pathway primarily uses dsDNA donors and requires RAD51 and BRCA2, like canonical HDR at DSBs. The alternative pathway uses ssDNA or nicked dsDNA donors, and is inhibited by RAD51 and BRCA2, but requires BRCA1. In cells treated with siBRCA2 or siRAD51, alternative HDR efficiently processes nicks targeted by the CRISPR/Cas9^{D10A} nickase, with little accompanying mutagenic end joining (mutEJ), so this pathway is of practical utility for genome engineering by targeted gene correction. Alternative HDR at nicks can be stimulated in physiological contexts in which canonical HDR is compromised by mutation or downregulated in response to environmental conditions or drugs, and can be one source of loss-of-heterozygosity (LOH), a common form of genomic instability in tumors.

Results

HDR is More Efficient at a Transcribed Strand Nick.

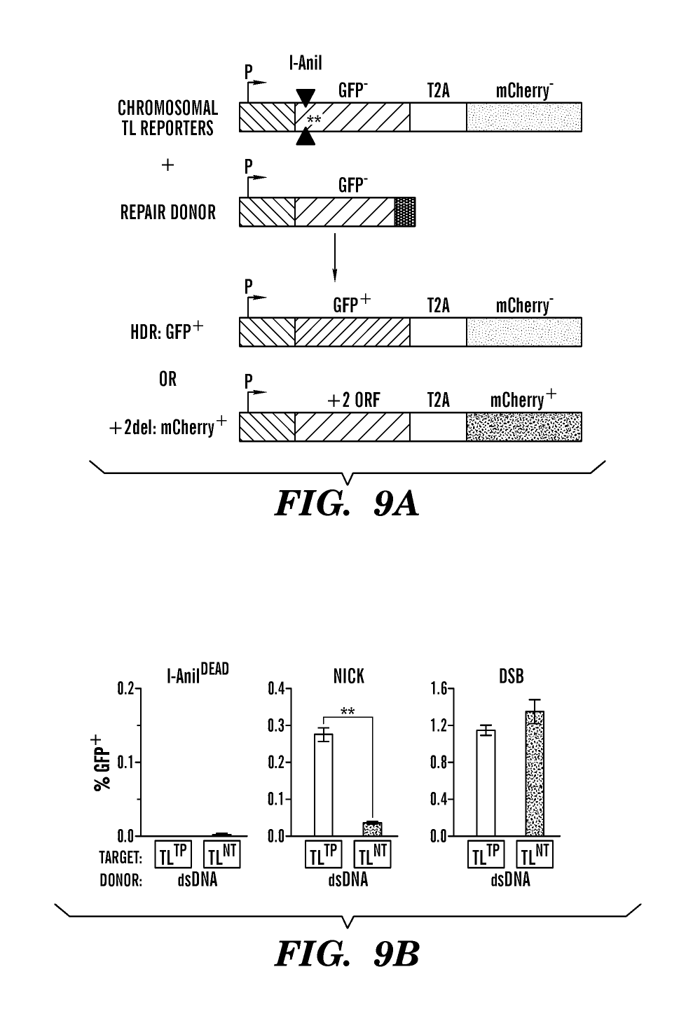

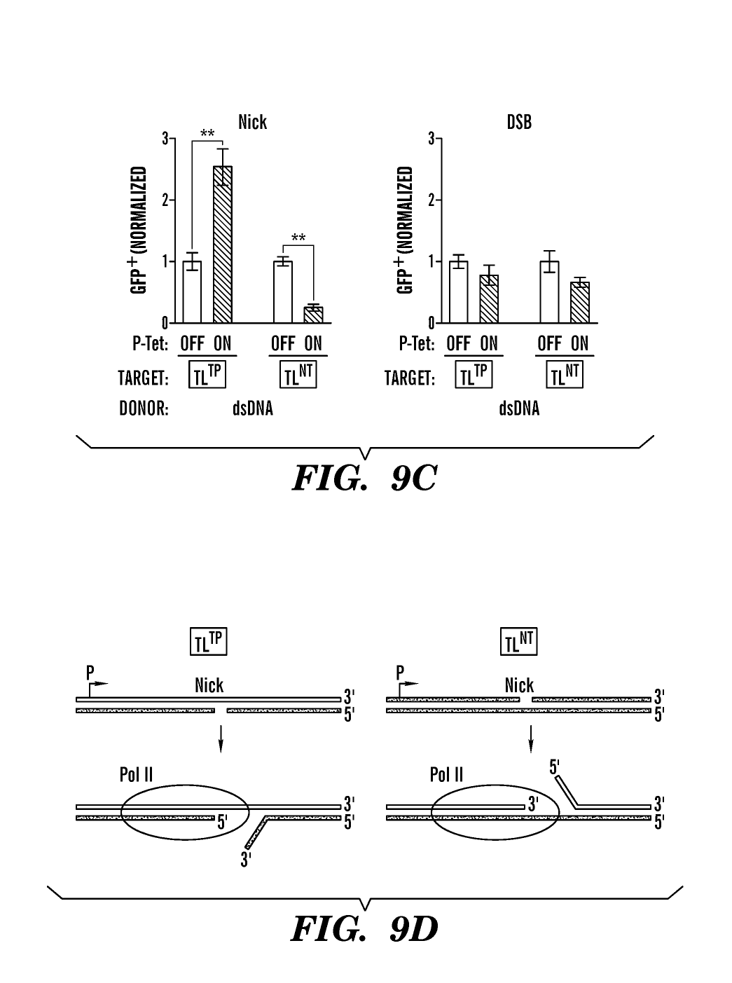

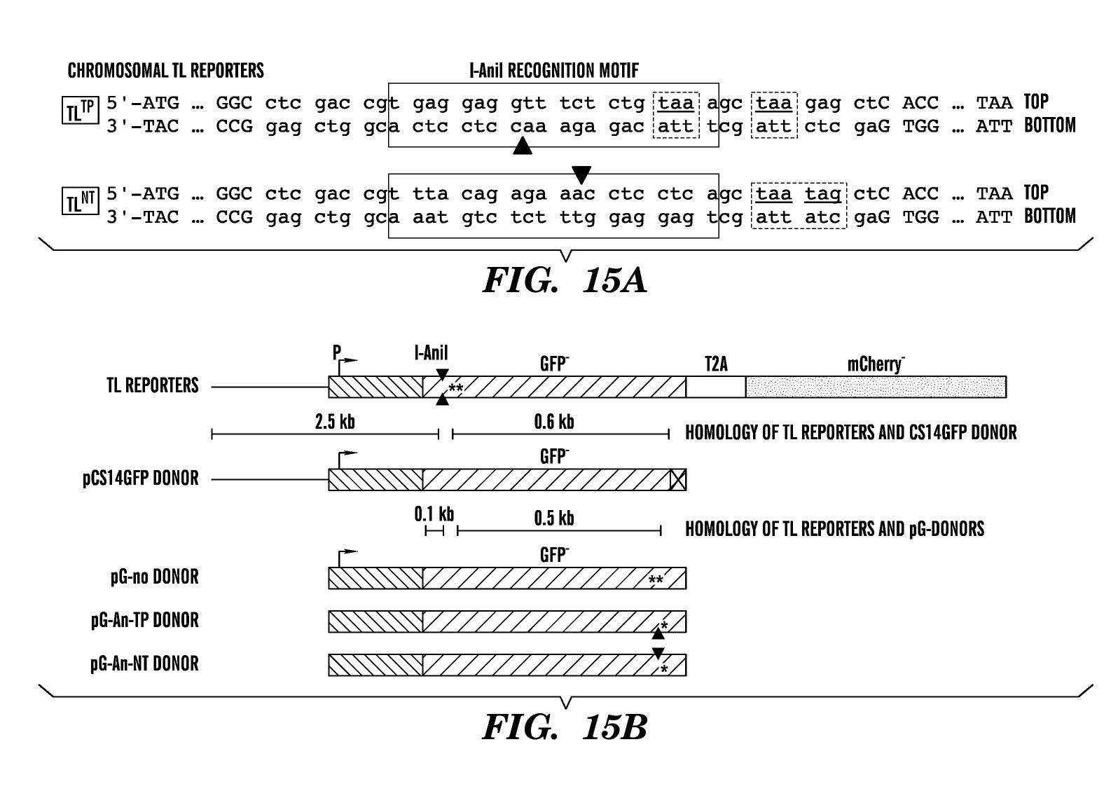

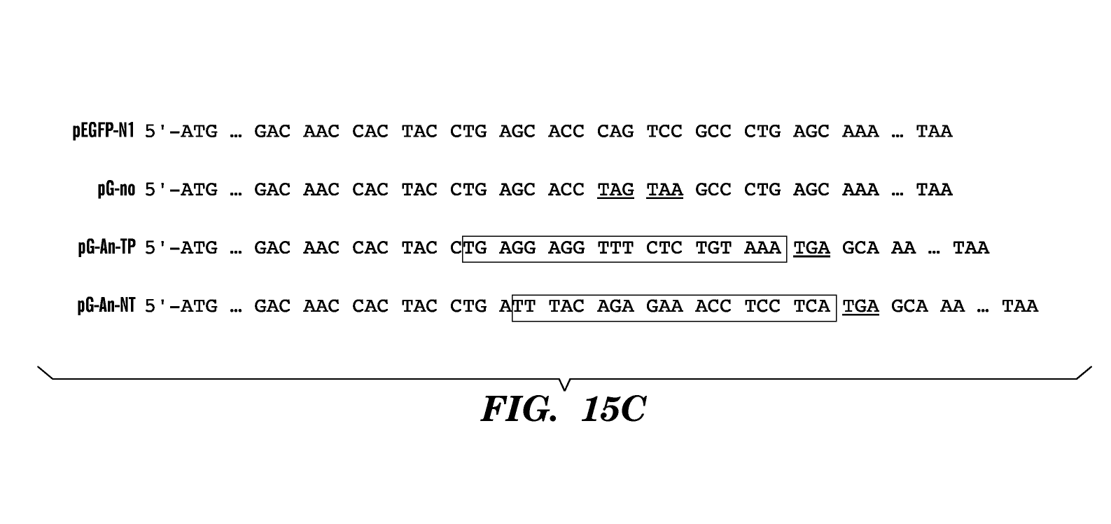

Damage to the transcribed strand is preferentially detected and repaired in transcription-coupled nucleotide excision repair (16), and transcribed strand nicks can arrest transcriptional elongation in human cell extracts (17). To ask if nick-initiated HDR at a transcribed gene exhibited similar strand bias, a “nickase” derivative of the I-AniI homing endonuclease, disabled at one of its two active sites so it cleaves only one DNA strand, generating a nick rather than a DSB (12) was used. An I-Ani-I site was inserted into the TL reporter (18) in both forward and reverse orientations, to create the TLTP and TLNT reporters, which support nicking on the transcribed or non-transcribed strand, respectively (FIG. 15A-15C). In cells bearing these reporters stably integrated in the genome as HDR by a homologous donor that replaces the I-AniI site and proximal stop codons yields GFP+ cells, while mutEJ events that cause a +2 frameshift yield mCherry+ cells (FIG. 9A).

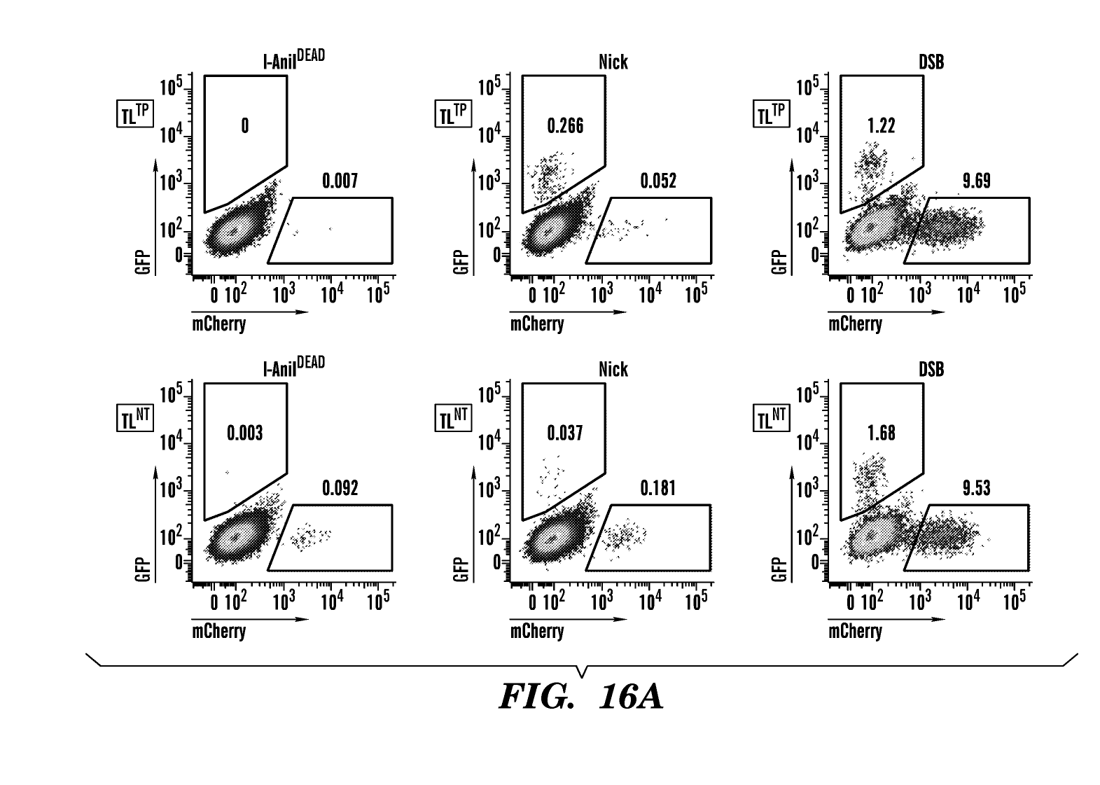

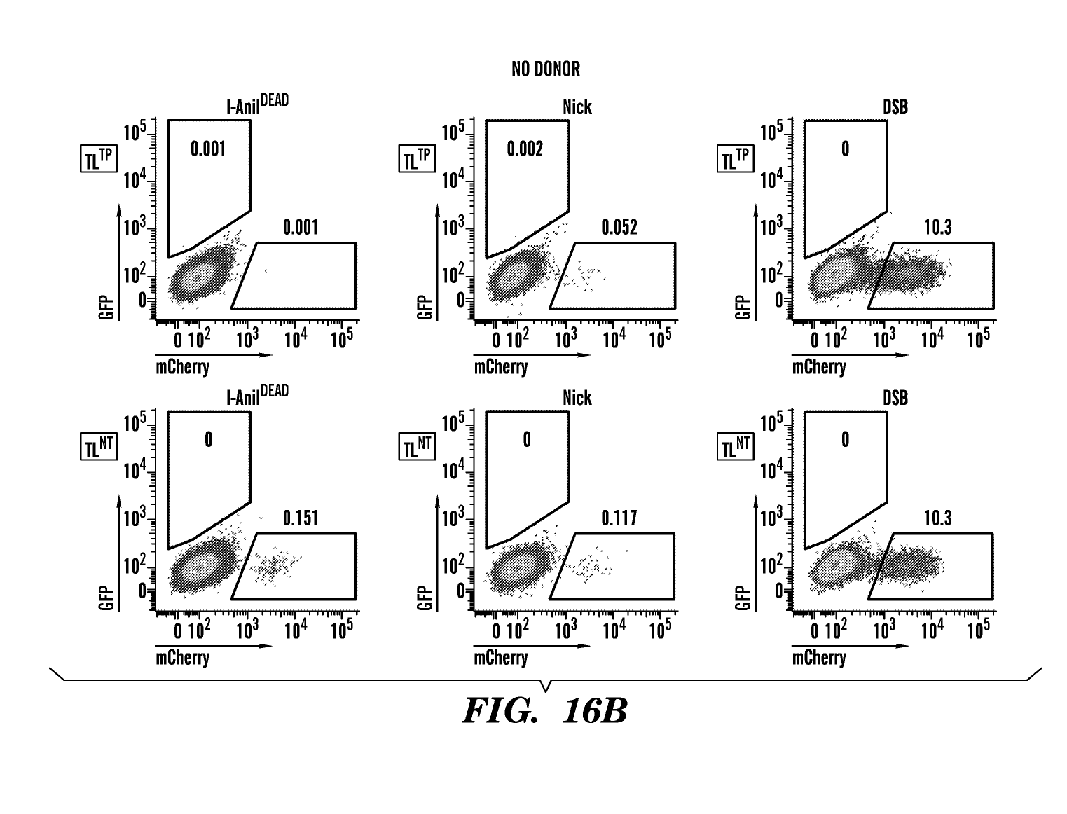

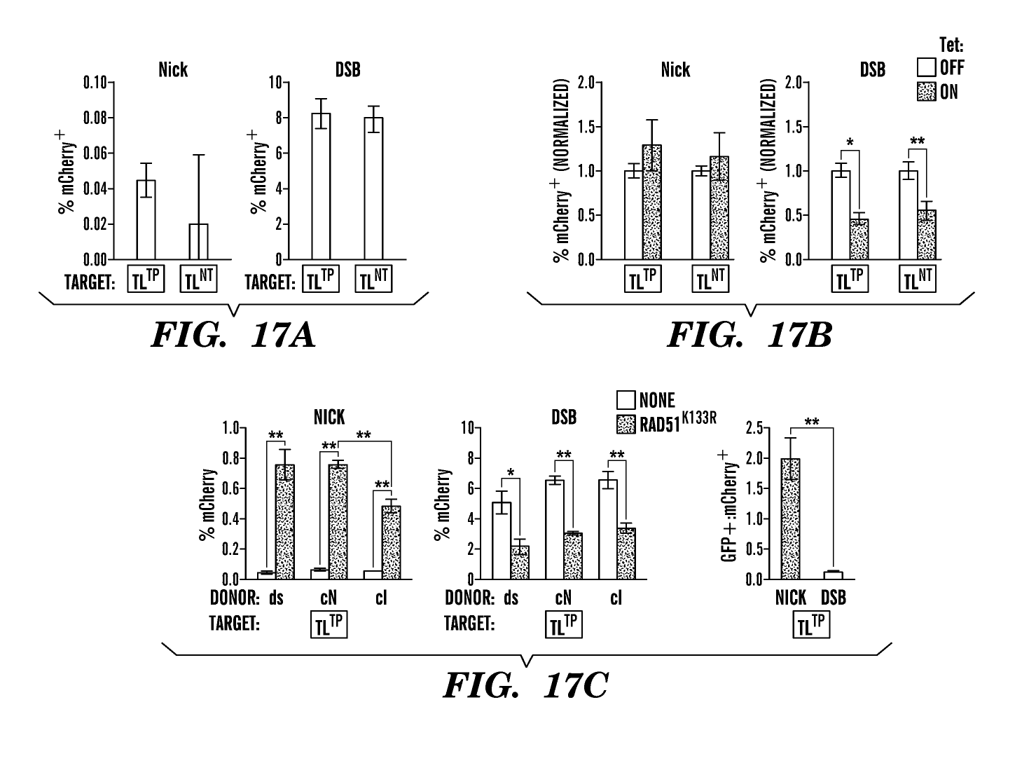

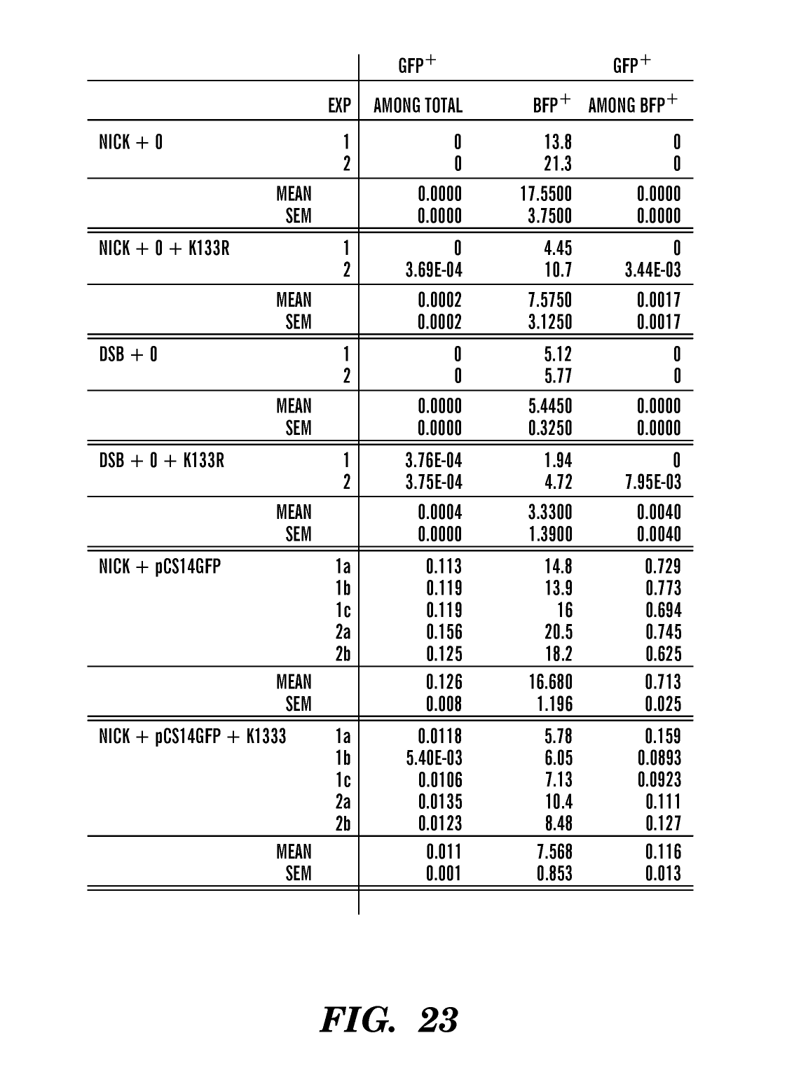

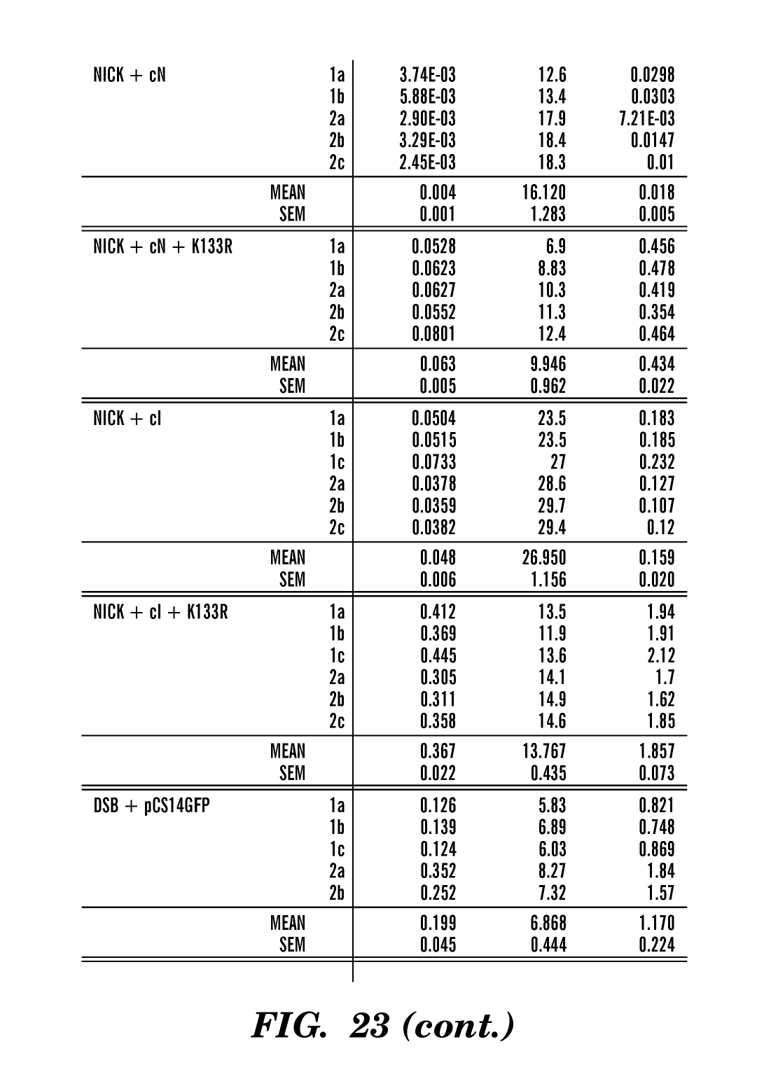

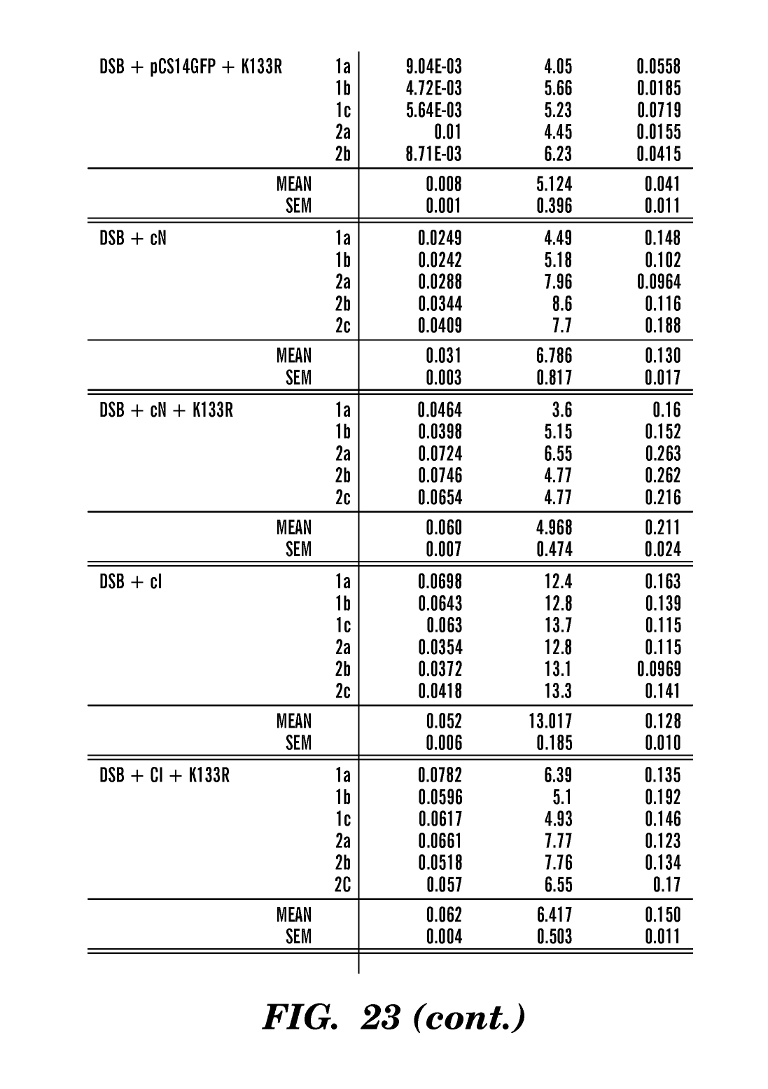

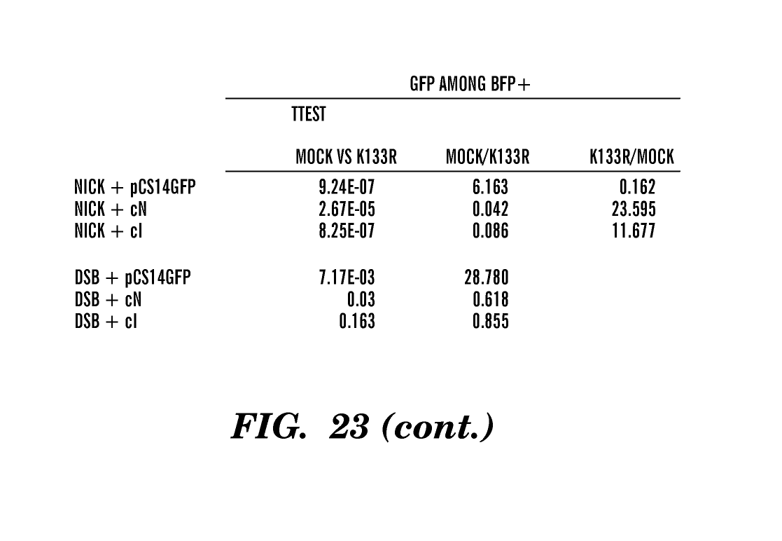

Populations of 293T cells bearing either the TLTP or TLNT reporter at heterogeneous integration sites were transiently transfected with a construct co-expressing I-AniI and the blue fluorescent protein mTagBFP (BFP), and with the dsDNA plasmid donor pCS14GFP. This donor is homologous with the TLTP and TLNT reporters over a region extending 2.47 kb upstream and 0.56 kb downstream of the I-AniI site (FIG. 15A-15C). GFP+ cells among I-AniI-expressing (BFP+) cells were quantified at 3 days post-transfection. No GFP+ cells were generated following expression either of catalytically inactive I-AniI in the presence of donor (FIGS. 9B and 16A) or of active I-AniI in the absence of donor DNA (FIGS. 16B and 23). Nicks initiated HDR with nearly 8-fold greater frequency in the TLTP than in the TLNT population. DSBs initiated HDR at comparable frequencies in both populations (FIG. 9B), indicating that I-AniI recognizes its site in the two reporters with comparable efficiency. Nicks initiated many fewer mutEJ events than did DSBs, and mutEJ frequencies were comparable in TLTP and TLNT populations (FIG. 17A). Thus, HDR at nicks exhibited a clear transcriptional strand bias: a transcribed strand nick is more efficiently repaired than a non-transcribed strand nick.

Transcription Stimulates HDR at a Transcribed Strand Nick and Inhibits HDR at a Non-Transcribed Strand Nick.

To ask if active transcription was required for the transcribed strand bias in HDR at nicks, derivatives of the TLTP and TLNT reporters were created in which a tetracycline-inducible (P-Tet) was substituted for the constitutive SFFV promoter upstream of GFP. TheseP-Tet TLTP and P-Tet TLNT reporters were stably integrated at the unique FRT site in Flp-In™ T-REx™-293 cells. Cells were cultured with (ON) or without (OFF) 1 μg/ml doxycycline, transfected with an I-AniI expression construct and the pCS14GFP plasmid dsDNA donor, and after 8-9 days of culture, doxycycline was added to the OFF cells to permit detection of HDR (GFP+) and mutEJ (mCherry+) events that had occurred in the absence of transcription. Active transcription increased the frequency of HDR at a transcribed-strand nick 2.5-fold, but reduced the frequency of HDR at a non-transcribed strand nick 4-fold (FIG. 9C). These opposing effects together accounted for the 8-fold greater frequency of HDR at a transcribed strand nick in a transcribed gene (FIG. 9B). Transcription did not affect the frequency of HDR at a DSB (FIG. 9C), consistent with other reports (19-21). Without wishing to be bound by theory, the transcription-associated strand bias of HDR at nicks may reflect unwinding ahead of the transcription apparatus that exposes the recombinogenic 3′ end of a transcribed strand nick that stimulates HDR, while occlusion of the 3′ end and exposure of the less recombinogenic 5′ end of a non-transcribed strand nick may hinder HDR (FIG. 9D). Similarly, the 2-fold reduction in mutEJ observed at DSBs, but not at nicks (FIG. 17B), may be caused by occlusion of the DSB by factors associated with the transcription apparatus.

HDR at Nicks with ssDNA Donors Displays Donor Strand Bias.

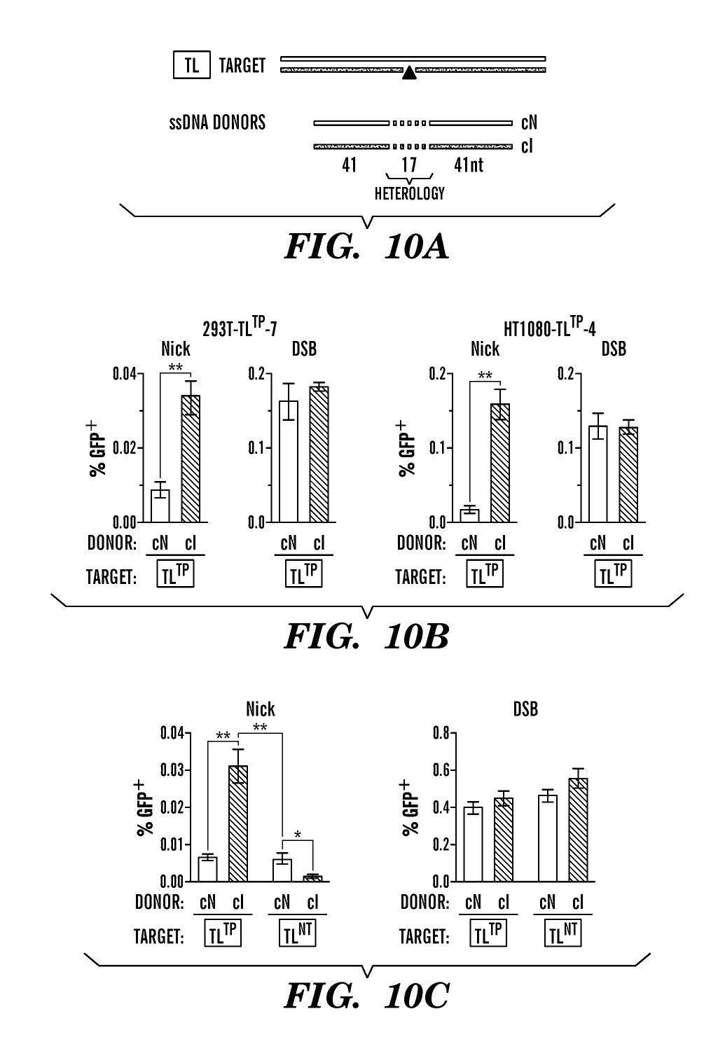

ssDNA molecules can serve as donors for HDR at DSBs (22-24). It was asked if they can also serve as donors for HDR at nicks using 99 nt ssDNA oligonucleotides in which a central 17 nt heterologous region corrects the mutations in the defective target gene (FIG. 10A). HDR at transcribed-strand nicks and DSBs was assayed in clonal derivatives of either 293T or HT1080 cells carrying the TLTP reporter (FIG. 10B). ssDNA donors complementary to either the intact (cI) or nicked (cN) DNA strand supported HDR, but the donor complementary to the intact, non-transcribed, strand was several-fold more efficient. No donor strand bias was evident at DSBs.

To learn what determines ssDNA donor strand bias, HDR by ssDNA donors complementary to the intact (cI) or nicked (cN) strand at transcribed and non-transcribed strand nicks in the TLTP and TLNT populations was compared. The TLTP and TLNT populations carry reporters delivered by lentiviruses and integrated at heterogeneous chromosomal positions, to minimize possible effects of replication direction on this assay. The ssDNA donor complementary to the intact strand supported HDR more efficiently regardless of whether the initiating nick was on the transcribed or non-transcribed strand, while no donor strand bias was evident in HDR at DSBs (FIG. 10C). These results show that donor strand bias is determined by whether the donor can anneal to the nicked or intact strand, and not by transcriptional orientation.

HDR at Nicks can Proceed Via an Alternative Pathway Normally Suppressed by RAD51 and BRCA2.

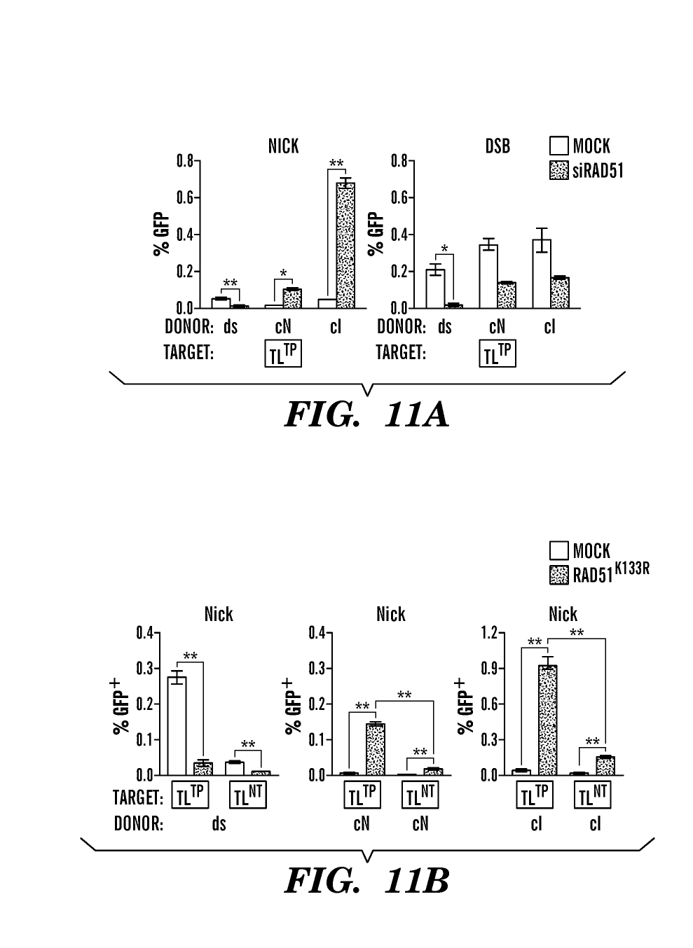

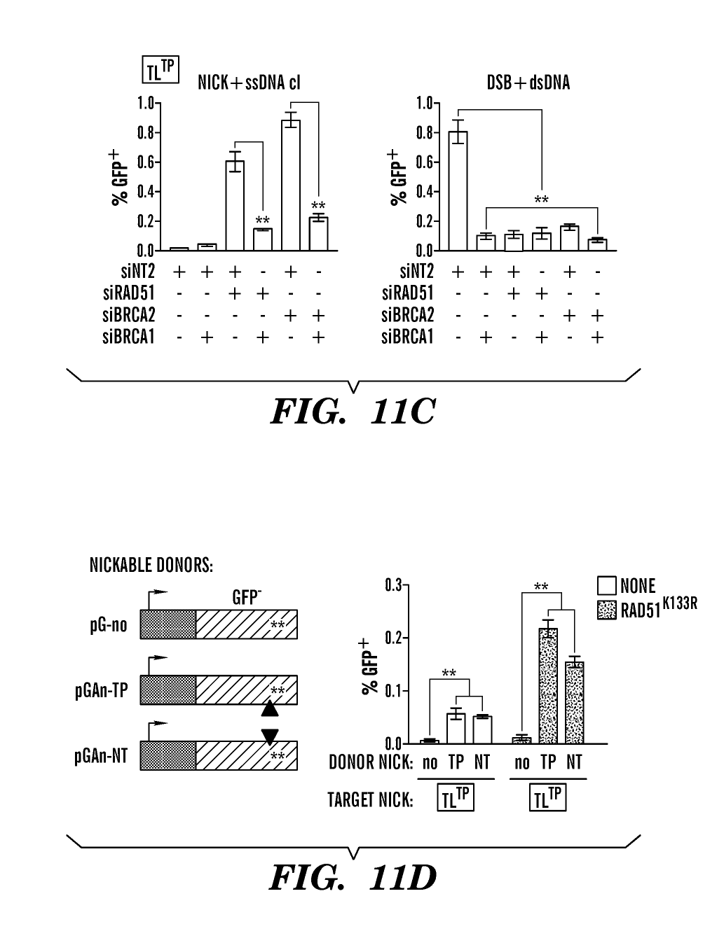

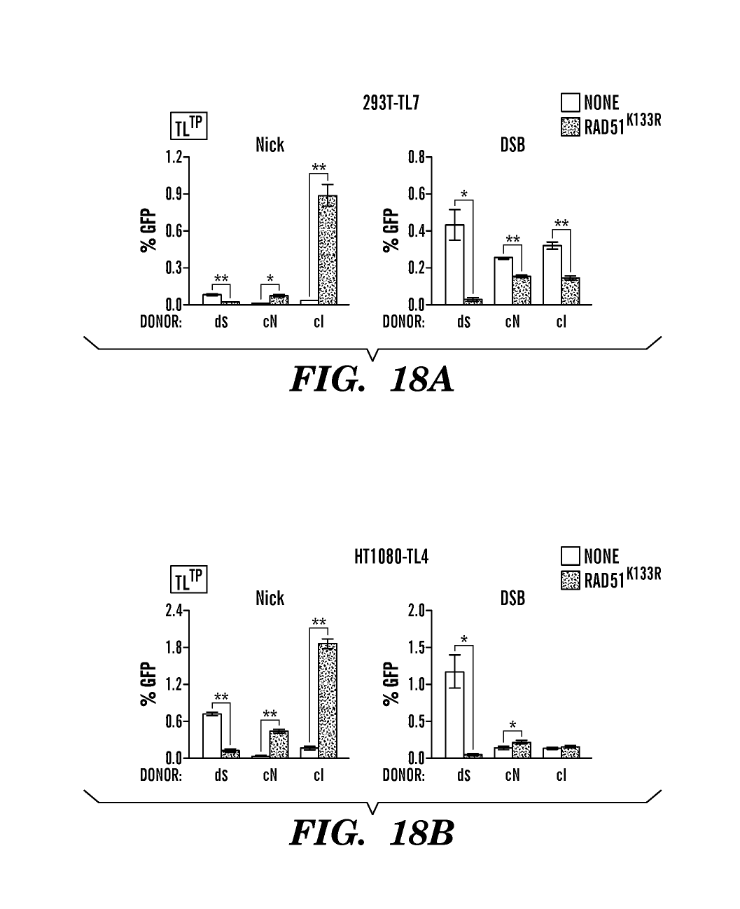

RAD51 promotes strand exchange and is a critical component of the canonical HDR pathway (15). The effect of RAD51 knockdown was examined by siRNA treatment of the clonal 293T-TL7TP line. Strikingly, siRAD51 greatly increased the frequency of HDR at nicks by ssDNA donors complementary to either strand, but not by a dsDNA donor (FIG. 11A). At DSBs, siRAD51 reduced the frequency of HDR, as expected, but had a much greater effect on HDR by a dsDNA than a ssDNA donor (FIG. 11A). Similar results were observed upon transient expression of RAD51K133R, a dominant negative mutant which does not hydrolyze ATP (25), in both the 293T-TL7TP and the HT1080-TL4TP clonal lines (FIGS. 18A and 18B). RAD51K133R expression had comparable effects in assays of TLTP and TLNT populations (FIG. 11B). It was noted that siRAD51 treatment (FIG. 11A) or RAD51K133R expression (FIGS. 18A-18B) reduced HDR at a DSB by a dsDNA donor more than 10-fold, but reduced HDR at a DSB by ssDNA donors 2-fold or less.

Single-strand annealing (SSA) in human cells repairs DSBs by joining flanking repeated sequences in cis, leading to deletion (23, 26-29). SSA is inhibited by RAD51 and by BRCA2, but requires BRCA1, prompting us to assay the effects of siBRCA2 and siBRCA1 on HDR at nicks. HDR at nicks using a ssDNA donor was stimulated 60-fold by siBRCA2 in 293T-TL7TP cells (FIG. 11C, left). siBRCA1 alone caused very modest stimulation (2.5-fold); but the stimulation of HDR observed in response to treatment with either siRAD51 or siBRCA2 was reduced 4-fold by siBRCA1 (FIG. 11C, left). At DSBs, either siBRCA1, siBRCA2 or siRAD51 inhibited HDR, as expected (FIG. 11C, right). This data indicated that HDR at nicks can proceed by an alternative pathway that is normally inhibited by the canonical RAD51/BRCA2-dependent HDR pathway, but requires BRCA1. Alternative HDR shares these features with the SSA pathway despite repairing different lesions (nicks vs. DSBs) and using distinct donors (ssDNA donors in trans vs. repetitive sequences in cis).

Nicked dsDNA Donors Promote Efficient Alternative HDR Pathway.

Concerted nicking of both donor and target DNA can stimulate HDR (30). To further define how the alternative HDR pathway depends on donor structure, HDR by intact or nicked dsDNA plasmid donors of a target nicked on the transcribed strand was compared. The donors carried a GFP gene that had been inactivated by insertion of two stop codons, and no I-AniI site (pG-no) or an I-AniI site at the 3′-end of GFP on either the transcribed (pGAn-TP) or non-transcribed (pGAn-NT) DNA strand to enable intracellular nicking in cells expressing I-AniI nickase (FIG. 11D, left and FIGS. 15A-15C). The nicked donors were more active than the intact donor, regardless of which donor strand was nicked, not only in cells carrying out canonical HDR but also in cells in which canonical HDR was suppressed by transient expression of RAD51K133R (FIG. 11D, right). Thus, genomic dsDNA that has been nicked in the course of replication, transcription, recombination or repair can serve as an intracellular donor for both canonical and alternative HDR at a nick in a homologous sequence.

Efficient Alternative HDR at Nicks Generated by CRISPR/Cas9D10A.

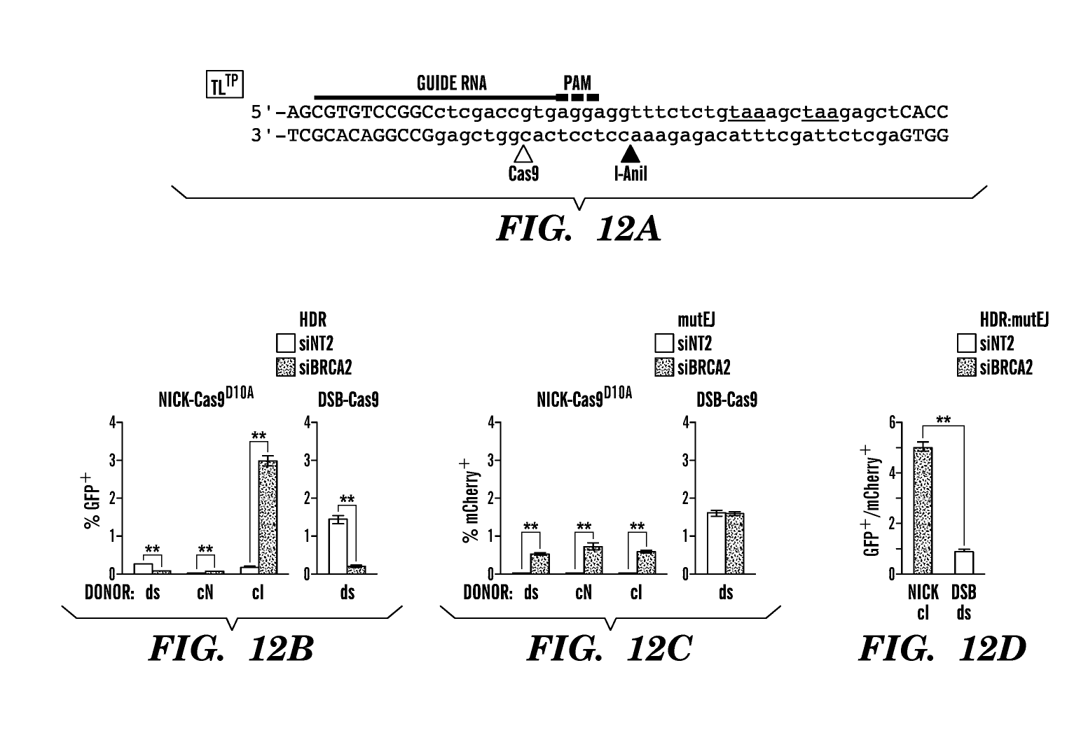

The very high efficiency of alternative HDR at nicks suggested that this pathway might be useful in targeted gene correction. The CRISPR/Cas9 system is ideal for this application because target specificity is easily modified, and, as with I-AniI, targeting by the nickase derivative CRISPR/Cas9D10A is accompanied by less local deletion than targeting by the CRISPR/Cas9 cleavase (31). To ask if nicks generated by CRISPR/Cas9D10A could be repaired by alternative HDR, Cas9D10A or Cas9WT was co-expressed with a CRISPR guide RNA designed to target the enzyme to a site 9 bp upstream of the I-AniI recognition sequence in the TLTP reporter (FIG. 12A) in 293T-TL7TP cells in which key canonical HDR factors were transiently inhibited by siRNA treatment, and HDR assayed (FIG. 12B). HDR with a dsDNA donor at either a nick or DSB was inhibited by siBRCA2; while HDR at a nick using a ssDNA donor was stimulated by siBRCA2. The ssDNA donor complementary to the intact strand (cI) supported HDR at levels 30-fold higher than the donor complementary to the nicked strand (cN). The effect of siBRCA2 treatment and the strand bias of the ssDNA repair donor were the same at nicks targeted by CRISPR/Cas9D10A and the I-AniI nickase.

The frequency of mutEJ (mCherry+ cells) at nicks generated by CRISPR/Cas9D10A was elevated by siBRCA2 treatment, but nonetheless significantly lower than the frequency of mutEJ at a DSB generated by CRISPR/Cas9WT (FIG. 12C). Moreover, the ratio of HDR:mutEJ at nicks by the alternative HDR pathway, assayed in siBRCA2-treated cells, was 5-fold higher than at DSBs by canonical HDR (FIG. 12D). Similar results were obtained in parallel assays of mutEJ initiated by I-AniI nickase in cells in which the alternative HDR pathway was stimulated by expression of dominant negative RAD51K133R (FIG. 17C). These data indicate that the key features of HDR at a nick via the alternative pathway are characteristic of the pathway and independent of the enzyme that targets the nick. It should therefore be straightforward to achieve very efficient gene targeting accompanied by low mutEJ in cells treated transiently with siBRCA2 to stimulate alternative HDR.

3′-5′ Unwinding or Resection of the Target May Promote HDR at a Nick.

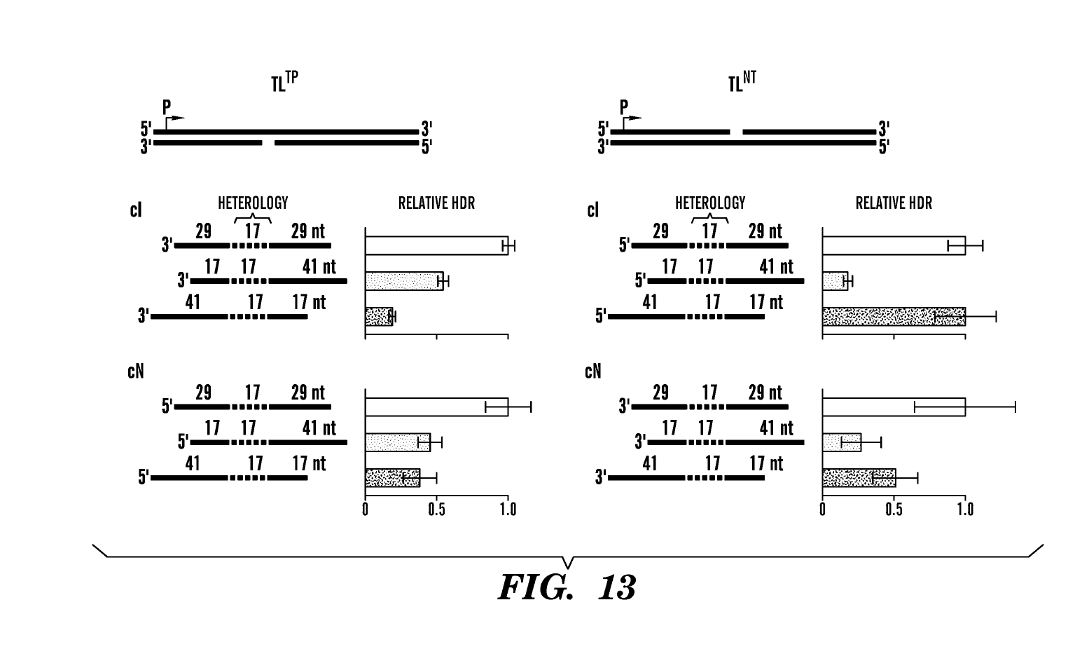

Donor DNA strands complementary to either the nicked or intact target strand are competent to engage the alternative HDR pathway (FIGS. 10A-10C and 11A-11D), suggesting an HDR mechanism in which unwinding or resection occurs at the nick to make both strands of the chromosomal target accessible for donor annealing. To determine whether there is a predominant directionality to unwinding or resection, a comparison was made of HDR at nicks by ssDNA donors identical in length (75 nt) and centered on the nick or extending either 3′ or 5′ of the nick. The donor centered on the nick was most efficient, but donors extending in either the 3′ or 5′ direction could support HDR, in both the TLTP and TLNT populations, with donors complementary to either the intact (cI) or nicked (cN) strand (FIG. 13). This indicates that HDR pathway involves unwinding or resection that exposes a gap on either side of the nick. However, HDR was least efficient if the region of extended homology was 3′ of the nick, suggesting that the predominant initial step may be unwinding or excision 5′ of the nick, by a helicase or a nuclease with 3′-5′ directionality. Without wishing to be bound by theory, the cN oligo works and also has this property of preferring 5′ extension, and it is thereof contemplated that the predominant 3′-5′ event is unwinding as resection/excision would remove homology for this donor.

Discussion

It is demonstrated herein that nicks that bypass the SSBR pathway may undergo HDR via two distinct pathways. One pathway requires RAD51 and BRCA2 and uses a dsDNA donor, like canonical DSB repair. The second, a novel alternative pathway, uses ssDNA or nicked dsDNA donors and is normally suppressed by RAD51 and BRCA2. Both canonical and alternative HDR were most efficient at a nick in the transcribed strand of a transcribed gene. HDR at nicks by the alternative pathway could be initiated by either I-AniI or CRISPR/Cas9D10A nickase. Alternative HDR at nicks is not only efficient but also accompanied by relatively little associated mutEJ, so it will be useful for genome engineering.

The data presented herein establish that HDR at nicks is distinct from HDR at DSBs in three ways. HDR at nicks—but not DSBs—is (1) transcription-associated; (2) preferentially uses a ssDNA donor complementary to the intact strand of the target; and (3) can proceed by an alternative HDR pathway that is stimulated by downregulation of RAD51 or BRCA2 expression or activity. Previous experiments had provided compelling evidence that a nick can initiate HDR (9-13), but left open the possibility that it might be necessary for a nick to be converted to a DSB for subsequent processing by the DSB repair pathway. The differences documented herein between HDR at nicks and DSBs make it very unlikely that a replicative DSB is an obligatory intermediate in HDR initiated by a nick.

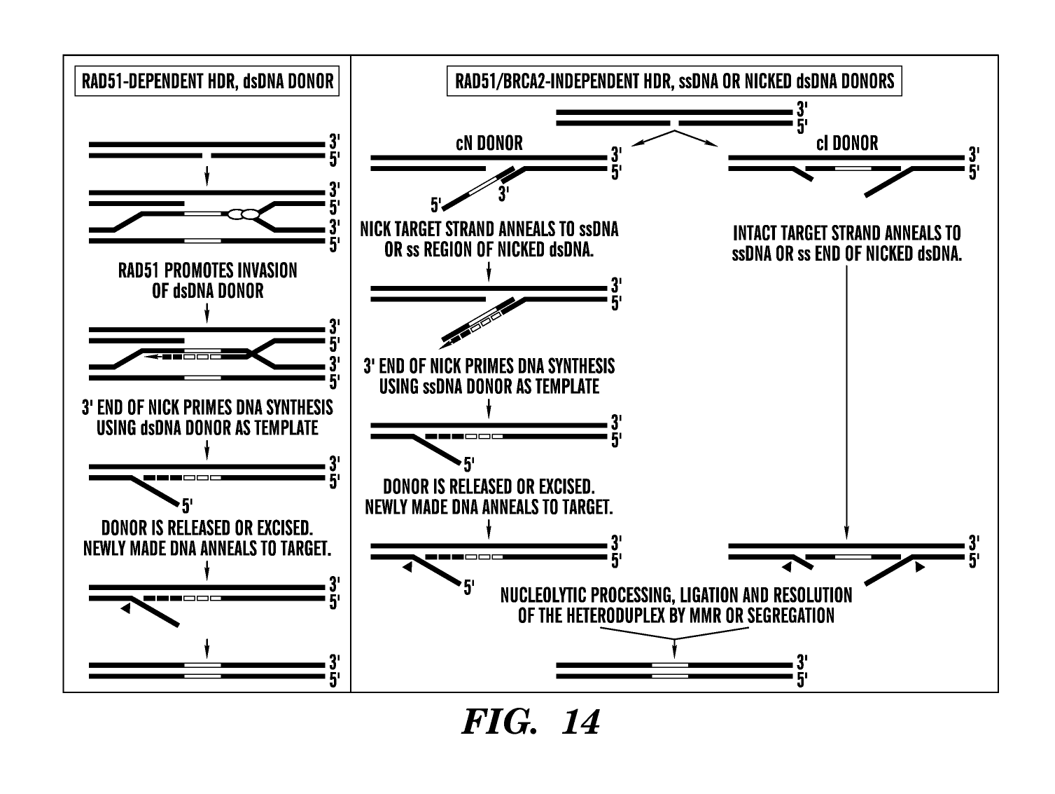

Without wishing to be bound by theory, the results reported here lead to the proposal of a working model for HDR at a nick (FIG. 14). In the first step, the flanking region is exposed to generate a gap. The apparent preference for a ssDNA donor with extended homology 3′ (rather than 5′) of the nick (FIG. 13) suggests that a helicase with 3′-5′ directionality may act at the nick to generate a free 3′ end. In HDR using a dsDNA donor (left), BRCA2 loads RAD51 on the free 3′ end to promote homology-dependent strand invasion, as in canonical DSB repair. This 3′ end is extended by repair DNA synthesis, again as in canonical DSB repair. The donor strand is then released and reanneals to the repair target, and flaps are removed and DNA ligated. This resembles gene conversion by synthesis-dependent strand annealing (15), but recombination might also involve crossover via a single Holiday junction intermediate (32, 33).

Use of a ssDNA donor occurs via an alternative HDR pathway, which is independent of RAD51/BRCA2 (FIG. 14, right). First, DNA unwinding or excision at the nick exposes a gap in the repair target; and then ssDNA anneals to the target. This step is not only independent of BRCA2 and RAD51 but strongly inhibited by these factors (FIGS. 11A-11D and 12A-12D).

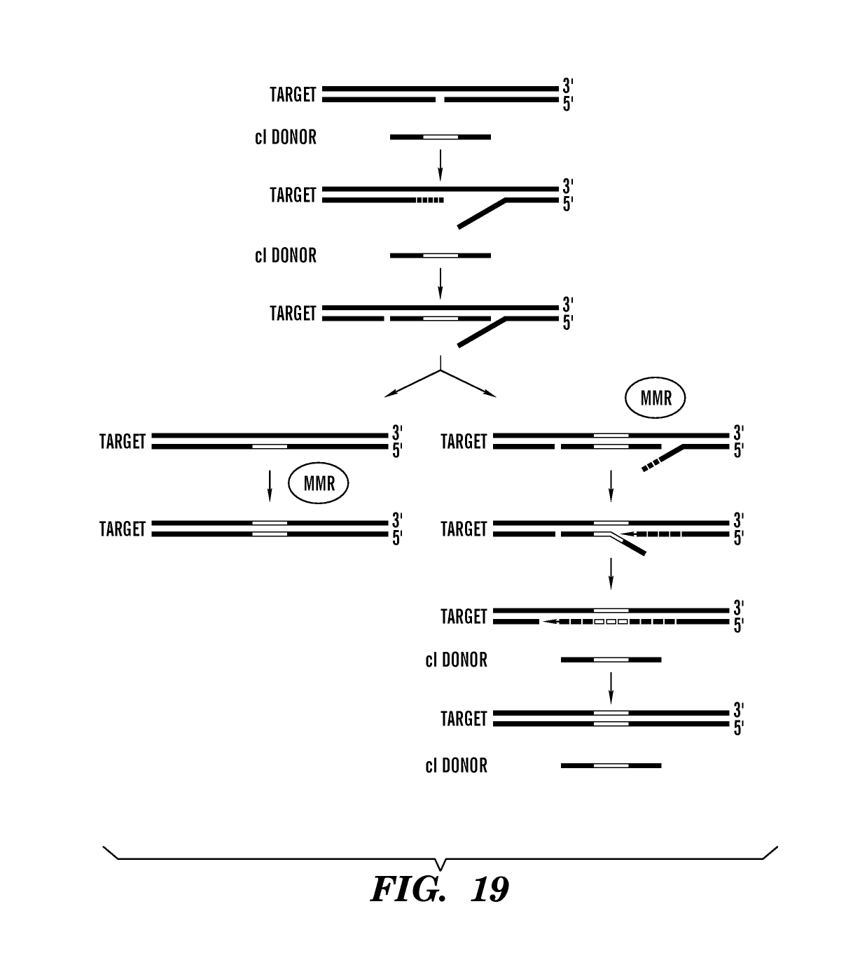

Inhibition may reflect the ability of BRCA2/RAD51 to drive recombination via the pathway that uses dsDNA donors (FIG. 14 left), which may compete with the alternative HDR pathway to carry out repair. This pathway can use donors complementary to either strand, albeit with differing efficiencies (FIGS. 10A-10C and 11A-11D), and subsequent events depend upon the strand used for repair. A donor complementary to the nicked strand (cN) can anneal to the free 3′-end of the target, and then serve as the template for repair synthesis primed by that 3′ end. Donor release then enables reannealing of the DNA duplex, followed by flap removal and ligation to complete HDR. Note the similarities with HDR at a nick using a dsDNA donor (FIG. 14, left), especially the requirement for 3′-5′ unwinding in both pathways. In the more efficient pathway, a donor complementary to the intact strand (cI) can anneal to the gap generated at the nick, forming a heteroduplex. The donor may then be ligated into the target (possibly requiring processing of donor or target ends), and heterology eliminated by mismatch repair or upon segregation (FIG. 14, left); or the donor may direct mismatch repair and be released in the course of repair synthesis (FIG. 19).

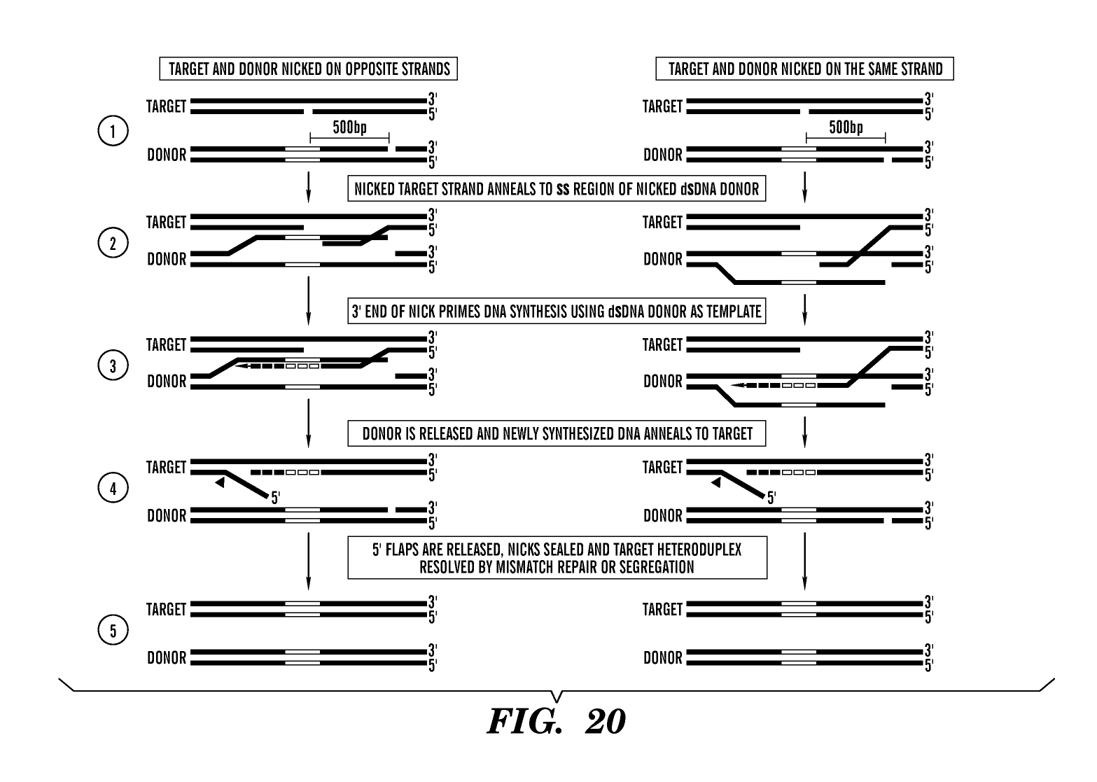

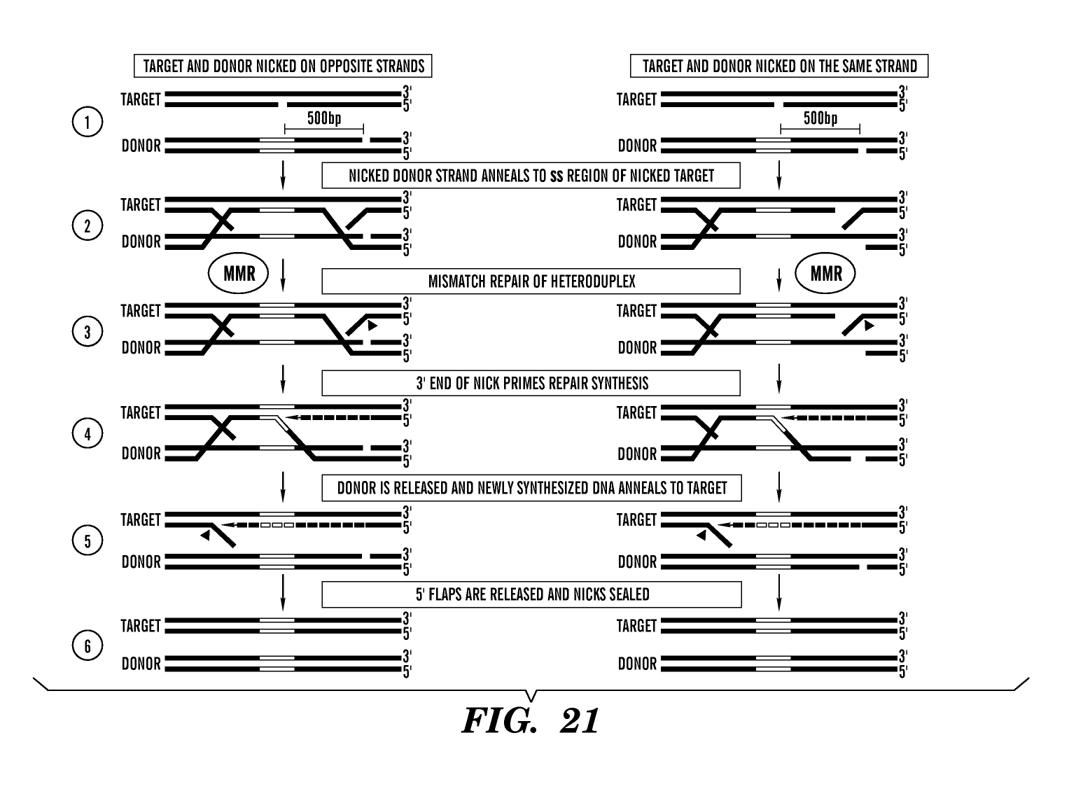

The pathways that support use of a ssDNA donor are also applicable to repair by a single-stranded region of a nicked dsDNA donor (FIGS. 20 and 21). In that context, this mechanism can be relevant to regulated diversification of immunoglobulin V regions by gene conversion (34), where cytidine deamination by Activation-Induced Deaminase (AID) is processed to generate a nick in the target, and repair is templated by upstream pseudogene donors. More generally, this mechanism can contribute to LOH. LOH without accompanying change in gene copy number occurs frequently in cancer cells and is an important source of mutations that drive tumorigenesis (35-37). LOH can occur if HDR uses an allelic region of the homologous chromosome as donor. If HDR between nicked homologs promotes LOH, then DNA nicks may constitute a more serious threat to genomic integrity than previously appreciated.

Alternative HDR at nicks is suppressed by canonical HDR, and can therefore be active in contexts in which canonical HDR is inactive. Examples include breast and ovarian cancers bearing BRCA2 mutations, and regions of solid tumors in which local hypoxic conditions downregulate canonical HDR (38-41). Recently, highly significant correlations have been documented between increased frequencies of loss of heterozygosity (LOH) and deficiencies in canonical HDR in primary breast and ovarian tumors and cell lines (42). This otherwise paradoxical observation may be explained if the alternative HDR pathway mediates LOH in these tumors, using the nicked homologous chromosome as donor.

Materials and Methods

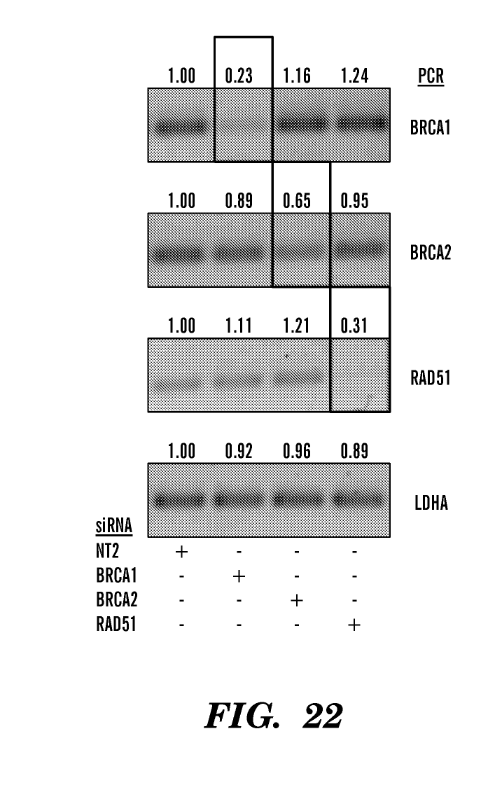

Cell culture and transfection. Human cells lines were cultured as described (13). siRNA and DNA transfections were performed according to the manufacturer's protocol (Lipofectamine RNAiMAX and LTX, repectively; Life Technologies, Grand Island, N.Y.). PCR of cDNA was used to determine the efficiency of siRNA knockdown (FIG. 22).

Flow cytometry, HDR and mutEJ frequencies. Cells were processed for flow cytometry as described previously (13). In experiments with I-AniI, which was co-expressed with mTagBFP (BFP), data are presented as GFP+ and mCherry+ frequencies among BFP+ cells, except in cases where flow was carried out more than 8 days post-transfection, by which time the BFP signal was largely extinguished. HDR and mutEJ frequencies were displayed as mean and standard error of the mean (SEM).

### REFERENCES

1. Caldecott K W (2008) Single-strand break repair and genetic disease Nat Rev Genet 9:619-631.
2. Evans M D, Dizdaroglu M, & Cooke M S (2004) Oxidative DNA damage and disease: induction, repair and significance Mutat Res 567:1-61.
3. Loeb L A & Harris C C (2008) Advances in chemical carcinogenesis: a historical review and prospective Cancer Res 68:6863-6872.
4. Foti J J, Devadoss B, Winkler J A, Collins J J, & Walker G C (2012) Oxidation of the guanine nucleotide pool underlies cell death by bactericidal antibiotics Science 336:315-319.
5. Gao Y, Katyal S, Lee Y, Zhao J, Rehg J E, Russell H R, & McKinnon P J (2011) DNA ligase III is critical for mtDNA integrity but not Xrcc1-mediated nuclear DNA repair Nature 471:240-244.
6. Katyal S & McKinnon P J (2011) Disconnecting XRCC1 and DNA ligase III Cell Cycle 10:2269-2275.
7. Simsek D, Furda A, Gao Y, Artus J, Brunet E, Hadjantonakis A K, Van Houten B, Shuman S, McKinnon P J, & Jasin M (2011) Crucial role for DNA ligase III in mitochondria but not in Xrcc1-dependent repair Nature 471:245-248.
8. Hanssen-Bauer A, Solvang-Garten K, Gilljam K M, Torseth K, Wilson D M, 3rd, Akbari M, & Otterlei M (2012) The region of XRCC1 which harbours the three most common nonsynonymous polymorphic variants, is essential for the scaffolding function of XRCC1 DNA Repair (Amst) 11:357-366.
9. Strathern J N, Weinstock K G, Higgins D R, & McGill C B (1991) A novel recombinator in yeast based on gene II protein from bacteriophage f1 Genetics 127:61-73.
10. Arcangioli B (1998) A site- and strand-specific DNA break confers asymmetric switching potential in fission yeast EMBO J 17:4503-4510.
11. Lee G S, Neiditch M B, Salus S S, & Roth D B (2004) RAG proteins shepherd double-strand breaks to a specific pathway, suppressing error-prone repair, but RAG nicking initiates homologous recombination Cell 117:171-184.
12. McConnell Smith A, Takeuchi R, Pellenz S, Davis L, Maizels N, Monnat R J, Jr., & Stoddard B L (2009) Generation of a nicking enzyme that stimulates site-specific gene conversion from the I-AniI LAGLIDADG (SEQ ID NO: 218) homing endonuclease Proc Natl Acad Sci USA 106:5099-5104.
13. Davis L & Maizels N (2011) DNA nicks promote efficient and safe targeted gene correction PLoS One 6:e23981.
14. Ramirez C L, Certo M T, Mussolino C, Goodwin M J, Cradick T J, McCaffrey A P, Cathomen T, Scharenberg A M, & Joung J K (2012) Engineered zinc finger nickases induce homology-directed repair with reduced mutagenic effects Nucleic Acids Res 40:5560-5568.
15. Heyer W D, Ehmsen K T, & Liu J (2010) Regulation of homologous recombination in eukaryotes Annu Rev Genet 44:113-139.
16. Hanawalt P C & Spivak G (2008) Transcription-coupled DNA repair: two decades of progress and surprises Nat Rev Mol Cell Biol 9:958-970.
17. Kathe S D, Shen G P, & Wallace S S (2004) Single-stranded breaks in DNA but not oxidative DNA base damages block transcriptional elongation by RNA polymerase II in HeLa cell nuclear extracts J Biol Chem 279:18511-18520.
18. Certo M T, Ryu B Y, Annis J E, Garibov M, Jar]our J, Rawlings D J, & Scharenberg A M (2011) Tracking genome engineering outcome at individual DNA breakpoints Nat Methods 8:671-676.
19. Taghian D G & Nickoloff J A (1997) Chromosomal double-strand breaks induce gene conversion at high frequency in mammalian cells Mol Cell Biol 17:6386-6393.
20. Allen C, Miller C A, & Nickoloff J A (2003) The mutagenic potential of a single DNA double-strand break in a mammalian chromosome is not influenced by transcription DNA Repair (Amst) 2:1147-1156.
21. Langston L D & Symington L S (2004) Gene targeting in yeast is initiated by two independent strand invasions Proc Natl Acad Sci USA 101:15392-15397.
22. Storici F, Durham C L, Gordenin D A, & Resnick M A (2003) Chromosomal site-specific double-strand breaks are efficiently targeted for repair by oligonucleotides in yeast Proc Natl Acad Sci USA 100:14994-14999.
23. Stark J M, Pierce A J, Oh J, Pastink A, & Jasin M (2004) Genetic steps of mammalian homologous repair with distinct mutagenic consequences Mol Cell Biol 24:9305-9316.
24. Chen F, Pruett-Miller S M, Huang Y, G]oka M, Duda K, Taunton J, Collingwood T N, Frodin M, & Davis G D (2011) High-frequency genome editing using ssDNA oligonucleotides with zinc-finger nucleases Nat Methods 8:753-755.
25. Stark J M, Hu P, Pierce A J, Moynahan M E, Ellis N, & Jasin M (2002) ATP hydrolysis by mammalian RAD51 has a key role during homology-directed DNA repair J Biol Chem 277:20185-20194.
26. Tuft A, Bertwistle D, Valentine J, Gabriel A, Swift S, Ross G, Griffin C, Thacker J, & Ashworth A (2001) Mutation in Brca2 stimulates error-prone homology-directed repair of DNA double-strand breaks occurring between repeated sequences EMBO J 20:4704-4716.
27. Ira G & Haber J E (2002) Characterization of RAD51-independent break-induced replication that acts preferentially with short homologous sequences Mol Cell Biol 22:6384-6392.
28. Weinstock D M, Richardson C A, Elliott B, & Jasin M (2006) Modeling oncogenic translocations: distinct roles for double-strand break repair pathways in translocation formation in mammalian cells DNA Repair (Amst) 5:1065-1074.
29. Bennardo N, Cheng A, Huang N, & Stark J M (2008) Alternative-NHEJ is a mechanistically distinct pathway of mammalian chromosome break repair PLoS Genet 4:e1000110.
30. Goncalves M A, van Nierop G P, Holkers M, & de Vries A A (2012) Concerted nicking of donor and chromosomal acceptor DNA promotes homology-directed gene targeting in human cells Nucleic Acids Res 40:3443-3455.
31. Mali P, Yang L, Esvelt K M, Aach J, Guell M, DiCarlo J E, Norville J E, & Church G M (2013) RNA-guided human genome engineering via Cas9 Science 339:823-826.
32. Cromie G A, Hyppa R W, Taylor A F, Zakharyevich K, Hunter N, & Smith G R (2006) Single Holliday junctions are intermediates of meiotic recombination Cell 127:1167-1178.
33. Smith G R (2004) How homologous recombination is initiated: unexpected evidence for single-strand nicks from v(d)j site-specific recombination Cell 117:146-148.
34. Maizels N (2005) Immunoglobulin gene diversification Annu Rev Genet 39:23-46.
35. Makishima H & Maciejewski J P (2011) Pathogenesis and consequences of uniparental disomy in cancer Clin Cancer Res 17:3913-3923.
36. Score J & Cross N C (2012) Acquired uniparental disomy in myeloproliferative neoplasms Hematol Oncol Clin North Am 26:981-991.
37. Pedersen B S & De S (2013) Loss of heterozygosity preferentially occurs in early replicating regions in cancer genomes Nucleic Acids Res 41:7615-7624.
38. Bindra R S, Schaffer P J, Meng A, Woo J, Maseide K, Roth M E, Lizardi P, Hedley D W, Bristow R G, & Glazer P M (2004) Down-regulation of Rad51 and decreased homologous recombination in hypoxic cancer cells Mol Cell Biol 24:8504-8518.
39. Meng A X, Jalali F, Cuddihy A, Chan N, Bindra R S, Glazer P M, & Bristow R G (2005) Hypoxia down-regulates DNA double strand break repair gene expression in prostate cancer cells Radiother Oncol 76:168-176.
40. Bristow R G & Hill R P (2008) Hypoxia and metabolism. Hypoxia, DNA repair and genetic instability Nat Rev Cancer 8:180-192.
41. Klein T J & Glazer P M (2010) The tumor microenvironment and DNA repair Semin Radiat Oncol 20:282-287.
42. Abkevich V, Timms K M, Hennessy B T, Potter J, Carey M S, Meyer L A, Smith-McCune K, Broaddus R, Lu K H, Chen J, et al. (2012) Patterns of genomic loss of heterozygosity
   predict homologous recombination repair defects in epithelial ovarian cancer Br J Cancer 107:1776-1782.

Supplemental Methods and Materials

Cell culture and transfection. The human embryonic kidney cell line HEK293T and Flp-In™ T-REx™-293 (Invitrogen), the human epithelial fibrosarcoma cell line HT1080 and their derivatives were grown at 37° C., 5% CO2 in Dulbecco-modified Eagle's medium (Hyclone) supplemented with 10% fetal bovine serum (Atlanta Biological, Lawrenceville, Ga.) and 200 units/ml penicillin, 200 μg/ml streptomycin (Hyclone) and 2 mM L-glutamine (Hyclone). TLTP and TLNT cell populations were created by lentiviral transduction using lentivirus derived from the TLTP and TLNT vectors (described below). TetON-TL cell lines were created by cotransfecting Flp-In™ T-REx™-293 cells with either pTetON-TLTP (described below) and pOG44 or with pTetON-TLNT (described below) and pOG44.

Transfections of expression plasmids and donor DNA were performed using Lipofectamine LTX™ (Life Technologies, Grand Island, N.Y.) according to the manufacturer's protocol. Briefly, transfection mixes consisted of approximately 1 μg of DNA and 2.5 μl of Lipofectamine LTX per 200 pl serum free DMEM or OptiMEM™ (Life Technologies). In experiments using I-AniI, transfecting DNA consisted of 300 ng I-AniI expression plasmid, 75 ng RAD51K133R expression plasmid where indicated, and either 500 ng (approximately 0.16 pmol) pCS14GFP dsDNA plasmid donor, or 0.6-0.7 pl of 33 pM (20 pmol) oligonucleotide donor per 200 pl serum free DMEM. In experiments using Cas9, transfecting DNA consisted of 250 ng Cas9 expression plasmid, 150 ng guide RNA expression plasmid and either 250 ng pCS14GFP dsDNA plasmid donor or 0.7 pl of 33 pM oligonucleotide donor per 200 pl OptiMEM™. When siRNA was not used, 293T or HT1080 cells were seeded at approximately 2×105 (293T) or 1×105 (HT1080) cells/ml and transfected the following day. In experiments not involving the TetON-TL reporter, cells were expanded 1 day post-transfection and collected for analysis 3 days post-transfection. For TetON-TL experiments, transfections were performed in either the presence or absence of 1 pg/ml doxycycline, cells were then expanded 1 day post-transfection and cultured for 8-9 more days in the presence or absence of doxycycline and then for one additional day in the presence of 1 pg/ml doxycycline prior to collection.

Transfections of siRNA in FIG. 11A and FIG. 18C were performed using Lipofectamine RNAiMAX™ (Life Technologies) according to the manufacturer's protocol. Briefly, 5.5×105 cells were seeded in 3 ml media in 6 cm plates (dl) and transfected (d2) with 60 pmol siRNA. Cells were split (d4) into 24- or 12-well plates at 1 or 2×105 cells/well, and 2-4 hr later were transfected a second time with siRNA; then transfected (d5) with expression plasmids and donor DNA, and expanded (d6) and collected for analysis (d8) as above.

Transfections of siRNA in FIGS. 11C and 12A-12D were performed using Lipofectamine RNAiMAX™ (Life Technologies). Briefly, on day 1, 4000 cells per well were plated in 0.1 ml media in a 96-well plate. On day 2, 10 pl of OptiMEM containing 0.125 pl RNAiMAX™ and 0.4 pl of 0.625 pM siRNA was added per well. On day 3, 20 pl of OptiMEM™ containing transfecting DNAs (expression constructs and donors, as above) and 0.2 μl of Lipofectamine LTX™ was added per well. Cells were collected for analysis on day 6, 3 days after transfection with DNAs.

siRNA used were as follows: NT2, RAD51 and BRCA2 siRNA (Life Technologies; 4390846, s11734 and s2085, respectively); BRCA1 (Qiagen; SI02664361 and SI02664368, pooled).

Flow cytometry. Cells were fixed in 2% formaldehyde and analyzed on an LSR II™ flow cytometer (Becton Dickinson, Franklin Lakes, N.J.). At least 100,000 events were gated for linear side scatter and forward scatter to identify cells, and cells gated for linear forward scatter height and width and side scatter height and width to eliminate doublets. In all experiments I-AniI was co-expressed with mTagBFP (BFP). Data are presented as GFP+ and mCherry+ frequencies among BFP+ cells, except in FIGS. 9D and 12B where late time points precluded meaningful measurements of BFP+ cells. GFP, mCherry, and mTagBFP fluorescence were detected with 488 nm, 561 nm 406 nm and 641 nm lasers, respectively. Data were analyzed using FlowJo™ (Tree Star, Ashland, Oreg.) flow cytometry analysis software and frequencies were transferred to Microsoft Excel™ in which statistical significance was determined by two-tailed t-test (FIG. 23). In all experiments I-AniI was co-expressed with mTagBFP (BFP) and all experiments, except those involving the TetON-TL reporter or Cas9, are presented as GFP+ and mCherry+ frequencies among BFP+ cells. In the TLTP and TLNT cell populations, a small background population of mCherry+ cells was detected upon expression of catalytically inactive I-AniI (TLTP; 0.016% and TLNT; 0.107%); this background was subtracted to give the data presented in FIG. 12A.

Expression analysis of siRNA treated 293T-TL7TP cells. RNA was isolated from cells at 48 hrs post siRNA transfection using the RNeasy Plus Mini™ Kit (Qiagen, Valencia, Calif.). cDNAs were synthesized using the QuantiTect™ Reverse Transcription Kit (Qiagen), and used as template for PCR using primers directed against the indicated genes. Band intensities were analyzed using Image Lab™ Software (Bio-Rad, Hercules, Calif.).

Plasmids. I-AniI expression vectors (1) and the TLTP reporter plasmid (pCVL Traffic Light Reporter 1.1™ (Ani target) Efla Puro), dsDNA donor plasmid (pCVL SFFV d14GFP Donor [referred to here as pCS14GFP])(2), hCas9 and gRNA_Cloning Vector (3) were previously described. The TLNT vector was made by PCR of the TLTP reporter plasmid with primers TLR_Ani2opp and TLR_Ani2opp-r. The TetON-TLNT and TetON-TLTP plasmids were made by performing PCR on the TLNT and TLTP vectors using primers SalI-GFP-F and PacI-mCh-R. The resulting PCR products were cloned into XhoI/PacI digested pFTSH_SbfI-PacI, a version of pcDNATM5/FRT/TO. Derivatives of pEGFP-N1 (Invitrogen, Carlsbad, Calif.) were used as donor in experiments that used nickable donors. These were made by digesting pEGFP-N1 with Bpu10I and replacing the 15-nt between the two Bpu10I sites at the 3′ end of the gene with annealed oligos (Bpu10I_{—}2×STOP, Bpu10I_AniTP, Bpu10I_AniNT and there complements) creating pG-no, pGAn-TP and pGAn-NT. The RAD51K133R expression plasmid (pCMV-RAD51K133R-T2A-IFP-1.4) was constructed by amplifying the human RAD51 gene by RT-PCR using primers hRAD51-F1 and hRAD51-R1, cloning into PCR2.1 using the Zero Blunt® TOPO® PCR Cloning Kit (Invitrogen, Carlsbad, Calif.), and then introducing the K133R mutation by site directed mutagenesis using primer RAD51-QC(K133R) and its complement. RAD51K133R was amplified by PCR using primers SgflRAD51-F and RAD51MluI-R, cleaved with SgfI and MluI, and cloned in frame with the IFP1.4 far-red fluorescent protein (4) separated by a T2A linker. The guide RNA directed against the TLTP reporter was constructed by annealing oligos AniTrLt-gDNA1-F and AniTrLt-gDNA1-R then extending then using Pfu Turbo™ polymerase (Agilent, Santa Clara, Calif.). The extended oligos were cloned into the AflII digested gRNA_Cloning Vector using the Gibson Assembly™ Master Mix (New England Biolabs, Ipswich, Mass.). The Cas9D10A expression plasmid was derived from pCas9 by site directed mutagenesis using oligo Cas9_D10A-QC and its complement.

Donor oligonucleotides. Regions of heterology are in lower case letters. In experiments using the TLTP reporter, which is nicked on the transcribed strand, oligos referred to as cN are designated below as TOP and those referred to as cI are designated below as BOT. In experiments using the TLNT reporter, which is nicked on the non-transcribed strand, oligos referred to as cN are designated below as BOT and those referred to as cI are designated below as TOP.

99-TOP:
(SEQ ID NO: 174)
5′-TGGACGGCGACGTAAACGGCCACAAGTTCAGCGTGTCCGGCgagggc
gagggcgatgcCACCTACGGCAAGCTGACCCTGAAGTTCATCTGCACCAC
CG-3′
99-BOT:
(SEQ ID NO: 175)
5′-CGGTGGTGCAGATGAACTTCAGGGTCAGCTTGCCGTAGGTGgcatcg
ccctcaccctcGCCGGACACGCTGAACTTGTGGCCGTTTACGTCGCCGTC
CA-3′
75-TOP:
(SEQ ID NO: 176)
5′-TAAACGGCCACAAGTTCAGCGTGTCCGGCgagggtgagggcgatgcC
ACCTACGGCAAGCTGACCCTGAAGTTCA-3′
75-BOT:
(SEQ ID NO: 177)
5′-TGAACTTCAGGGTCAGCTTGCCGTAGGTGgcatcgccctcaccctcG
CCGGACACGCTGAACTTGTGGCCGTTTA-3′
75-TOPleft:
(SEQ ID NO: 178)
5′-TGGACGGCGACGTAAACGGCCACAAGTTCAGCGTGTCCGGCgagggt
gagggcgatgcCACCTACGGCAAGCTGA-3′
75-BOTleft:
(SEQ ID NO: 179)
5′-TCAGCTTGCCGTAGGTGgcatcgccctcaccctcGCCGGACACGCTG
AACTTGTGGCCGTTTACGTCGCCGTCCA-3′
75-TOPright:
(SEQ ID NO: 180)
5′-AGTTCAGCGTGTCCGGCgagggtgagggcgatgcCACCTACGGCAAG
CTGACCCTGAAGTTCATCTGCACCACCG-3′
75-BOTright:
(SEQ ID NO: 181)
5′-CGGTGGTGCAGATGAACTTCAGGGTCAGCTTGCCGTAGGTGgcatcg
ccctcaccctcGCCGGACACGCTGAACT-3′
BRCA1-RT1-F:
(SEQ ID NO: 182)
5′-GAGCTCGCTGAGACTTCCTG-3′
BRCA1-RT1-R:
(SEQ ID NO: 183)
5′-ACTCCAGACAGATGGGACACT-3′
BRCA2-RT1-F:
(SEQ ID NO: 184)
5′-GTTCCCTCTGCGTGTTCTCA-3′
BRCA2-RT1-R:
(SEQ ID NO: 185)
5′-CCATCCACCATCAGCCAACT-3′
RAD51-RT1-F:
(SEQ ID NO: 186)
5′-GCGAGTAGAGAAGTGGAGCG-3′
RAD51-RT1-R:
(SEQ ID NO: 187)
5′-TTAGCTCCTTCTTTGGCGCA-3′
LDHA-RT1-F:
(SEQ ID NO: 188)
5′-TCTTGACCTACGTGGCTTGG-3′
LDHA-RT1-R:
(SEQ ID NO: 189)
5′-AAGCACTCTCAACCACCTGC-3′

Other oligonucleotide sequences:

TLR_Ani2opp:
(SEQ ID NO: 190)
5′-gtttacagagaaacctcctcagctaatagctcacctacggc-3′
TLR Ani2opp-r:
(SEQ ID NO: 191)
5′-ctgaggaggtttctctgtaaacggtcgaggccggac-3′.
SalI-GFP-F:
(SEQ ID NO: 192)
5′-GTAGTCGACGCCACCATGGTGAGC-3′
PacI-mCh-R:
(SEQ ID NO: 193)
5′-GCATTAATTAAGAGCCTCTGCATTCACTTG-3′
Bpu10I_2xSTOP:
(SEQ ID NO: 194)
5′-tgagcacctagtaagccc-3′
Bpu10I_2xSTOP-R:
(SEQ ID NO: 195)
5′-tcagggcttactaggtgc-3′
Bpu10I_AniTL-TP:
(SEQ ID NO: 196)
5′-tgaggaggtttctctgtaaa-3′
Bpu10I_AniTL-TP-R:
(SEQ ID NO: 197)
5′-tcatttacagagaaacctcc-3′
Bpu10I_AniTL-NT:
(SEQ ID NO: 198)
5′-tgatttacagagaaacctcctca-3′
Bpu10I_AniTL-NT-R:
(SEQ ID NO: 199)
5′-tcatgaggaggtttctctgtaaa-3′
hRad51-F1:
(SEQ ID NO: 200)
5′-ATGGCAATGCAGATGCAG-3′
hRad51-R1:
(SEQ ID NO: 201)
5′-TCAGTCTTTGGCATCTCCC-3′
RAD51-QC(K133R):
(SEQ ID NO: 202)
5′-GGAGAATTCCGAACTGGGAgGACCCAGATCTGTCATACG-3′
SgfIRAD51-F:
(SEQ ID NO: 203)
5′-GCTTAAGGCGATCGCCATGGCAATGCAGATGCAGC-3′
RAD51MluI-R:
(SEQ ID NO: 204)
5′-GTAACGCGTGTCTTTGGCATCTCCCACTCC-3′
AniTrLt-gDNA1-F:
(SEQ ID NO: 205)
5′-tttcttggctttatatatcttgtggaaaggacgaaacaccggtgtcc
ggcctcgaccgtg-3′
AniTrLt-gDNA1-R:
(SEQ ID NO: 206)
5′-gactagccttattttaacttgctatttctagctctaaaaccacggtc
gaggccggacacc-3′
Cas9_D10A-QC:
(SEQ ID NO: 207)
5′-gaagtactccattgggctcgctatcggcacaaacagcgtc-3′

Supplemental Methods & Materials References
1. Davis L & Maizels N (2011) DNA nicks promote efficient and safe targeted gene
   correction PLoS One 6:e23981.
2. Certo M T, Ryu B Y, Annis J E, Garibov M, Jarjour J, Rawlings D J, & Scharenberg A M (2011) Tracking genome engineering outcome at individual DNA breakpoints Nat Methods 8:671-676.
3. Mali P, Yang L, Esvelt K M, Aach J, Guell M, DiCarlo J E, Norville J E, & Church G M (2013) RNA-guided human genome engineering via Cas9 Science 339:823-826.
4. Shu X, Royant A, Lin M Z, Aguilera T A, Lev-Ram V, Steinbach P A, & Tsien R Y (2009) Mammalian expression of infrared fluorescent proteins engineered from a bacterial phytochrome Science 324:804-807.

### Example 3

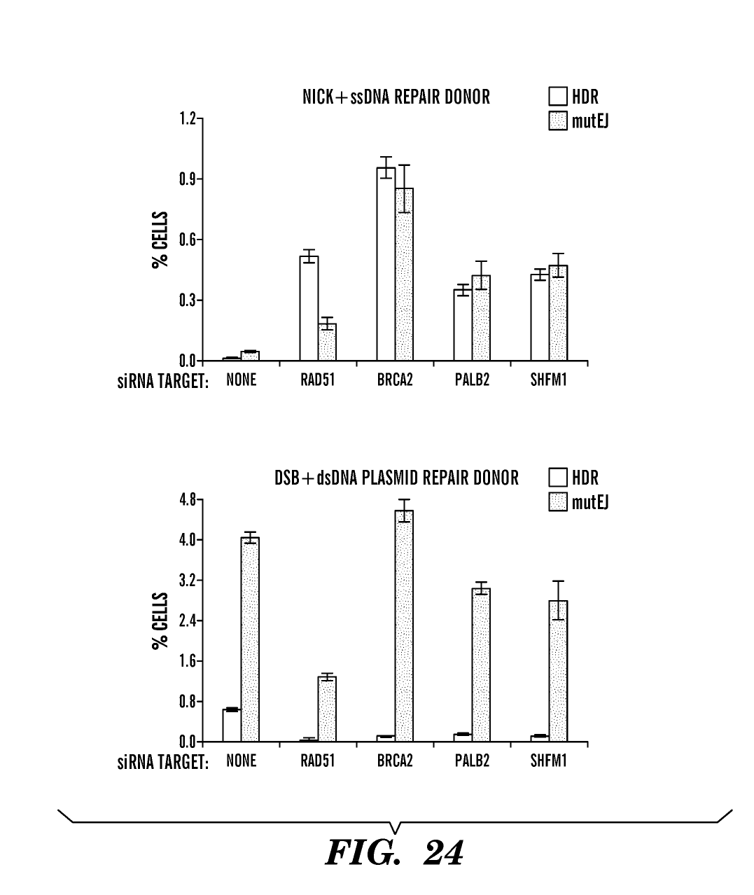

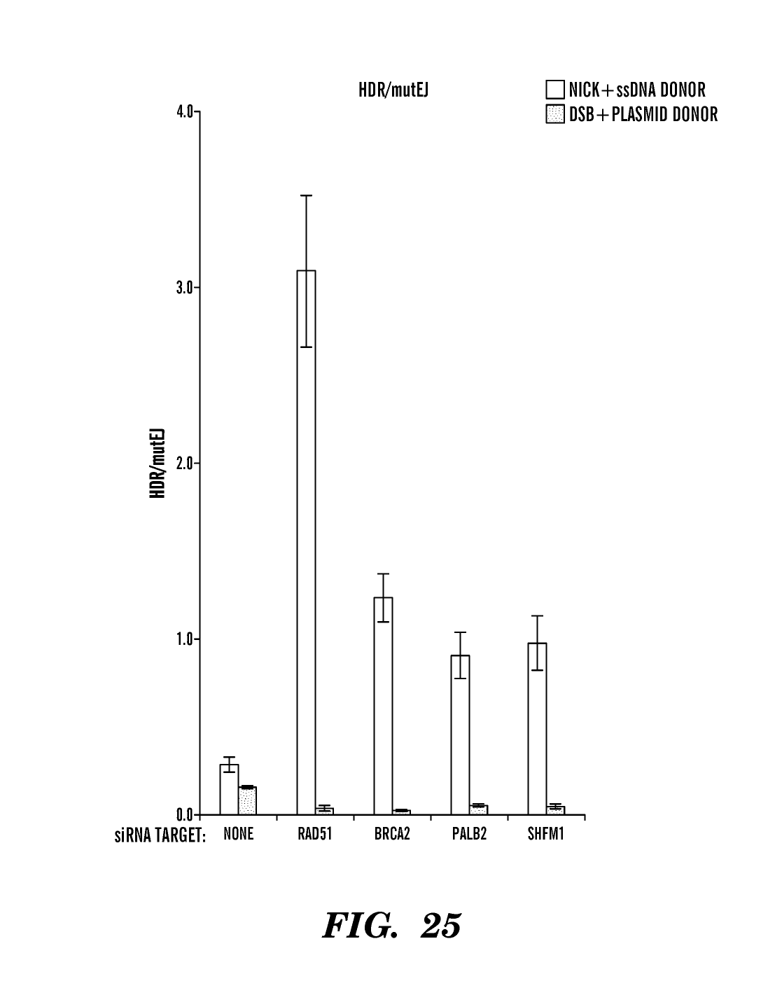

The frequency of homology-directed repair (HDR) at a nick using a ssDNA donor is increased upon siRNA knockdown of RAD51, BRCA2 or BRCA2-interacting factors PALB2 or SHFM1 to levels higher than that of HDR at a double-strand break (DSB) (FIG. 24). The ratio of HDR to mutEJ (local mutagenesis) is higher at a nick than at a DSB, in untreated cells and especially in cells in which RAD51, BRCA2 or BRCA2—interacting factors PALB2 or SHFM1 are knocked down by siRNA treatment (FIG. 25).

### Example 4

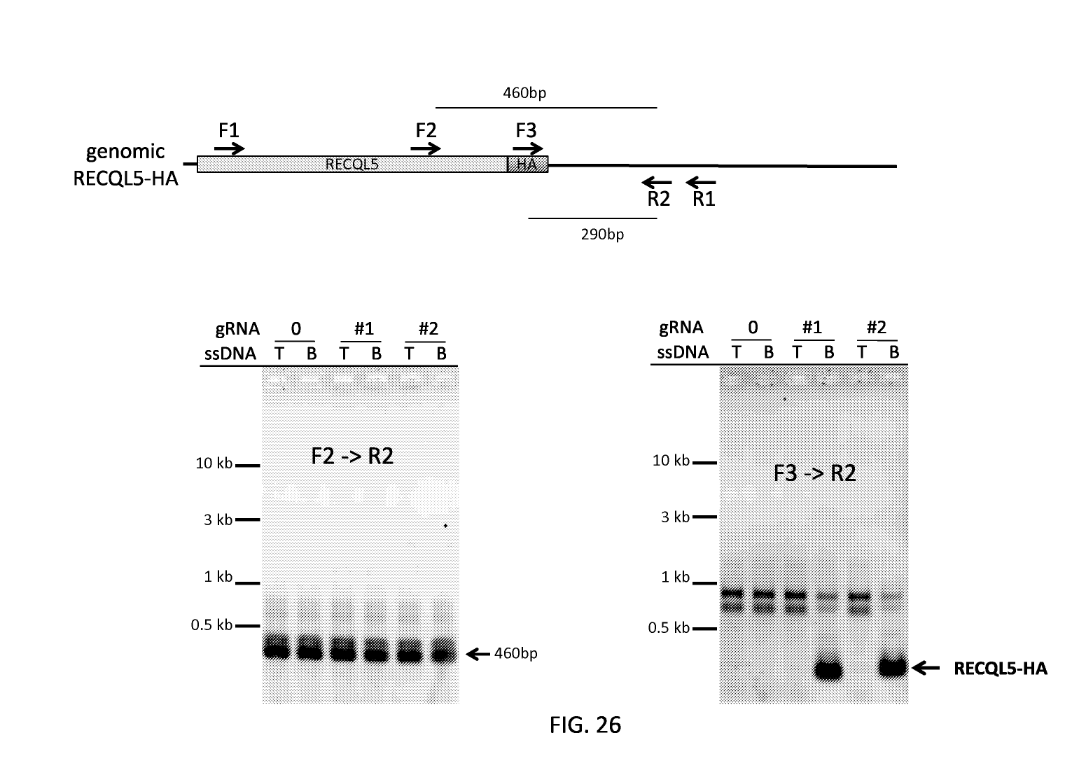

The tagging of endogenous RECQL5 using alternative HDR is depicted in FIG. 26. Using the methods described herein, an HA “tag” was added to the 3′ end of genomic RECQL5. Nested PCR demonstrated the successful insertion of the tag into the desired genomic locus.

1. (canceled)

2. A method of modifying the sequence of a target nucleic acid molecule, the method comprising contacting the target nucleic acid molecule with
a) a donor nucleic acid molecule comprising the modification to be made in the target nucleic acid molecule;
b) a nickase; and
c) an inhibitor of RAD51; BRCA2; PALB2 or SHFM1.

3. The method of claim 2, wherein a cell-free system comprises the target nucleic acid molecule.

4. The method of claim 2, wherein a cell comprises the target nucleic acid molecule.

5. (canceled)

6. (canceled)

7. (canceled)

8. The method of claim 2, wherein the nickase is selected from the group consisting of:
a nuclease with one active site disabled; I-AniI with one active site disabled; or Cas9^{D10A}.

9. The method of claim 2, wherein the donor nucleic acid molecule is a ssDNA or nicked dsDNA.

10. The method of claim 2, wherein the donor nucleic acid molecule comprises a portion complementary to the strand of the target nucleic acid molecule that is not nicked by the nickase.

11. The method of claim 10, wherein the portion of the donor nucleic acid molecule that is complementary to a strand of the target nucleic acid molecule is substantially centered with respect to the location of the nick.

12. A method of modifying the sequence of a target nucleic acid molecule, the method comprising contacting the target nucleic acid molecule with
a) a ssDNA donor nucleic acid molecule comprising the modification to be made in the target nucleic acid molecule;
b) a nuclease; and
c) an inhibitor of RAD51; BRCA2; PALB2 or SHFM1.

13. The method of claim 12, wherein a cell-free system comprises the target nucleic acid molecule.

14. The method of claim 12, wherein a cell comprises the target nucleic acid molecule.

15. (canceled)

16. (canceled)

17. The method of claim 12, wherein the donor nucleic acid molecule comprises a portion complementary to one strand of the target nucleic acid molecule.

18. The method of claim 12, wherein the nuclease is selected from the group consisting of:
nucleases comprising a FokI cleavage domain; zinc finger nucleases; TALE nucleases; RNA-guided engineered nucleases; Cas9; Cas9-derived nucleases; and homing endonucleases.

19. The method of claim 2, wherein the modification is introduced as a gene therapy.

20. The method of claim 2, wherein the inhibitor is an inhibitory nucleic acid; an antibody reagent; or selected from the group consisting of IBR2; RI-1; RI-2; and B02.

21. (canceled)

22. (canceled)

23. The method of claim 2, wherein the donor nucleic acid molecule is at least about 25 nt in length.

24. (canceled)

25. The method of claim 2, further comprising the step of implanting a cell comprising the modified nucleic acid molecule into a subject.

26. The method of claim 25, wherein the cell is autologous to the subject and/or is an iPS cell.

27. (canceled)

28. (canceled)

29. (canceled)

30. (canceled)

31. (canceled)

32. A method of decreasing genomic instability in a cell, the method comprising contacting the cell with an agonist of RAD51; BRCA2; PALB2 or SHFM1 or an inhibitor of BRCA1.

33. (canceled)

34. (canceled)

35. (canceled)

36. The method of claim 32, wherein the cell is a cancerous cell.

37. (canceled)

38. The method of claim 32, wherein the contacting step comprises administering the agonist or inhibitor to a subject in need of treatment for a risk of genomic instability.

39.-48. (canceled)
